(19) United States
(12) Patent Application Publication
(10) Pub. No.: US 2023/0043648 A1
(43) Pub. Date: Feb. 9, 2023

(54) COMPOUNDS AND METHODS FOR MODULATING SCN1A EXPRESSION

(71) Applicant: Ionis Pharmaceuticals, Inc., Carlsbad, CA (US)

(72) Inventors: Paymaan Jafar-Nejad, Carlsbad, CA (US); Susan M. Freier, San Diego, CA (US); Frank Rigo, Carlsbad, CA (US)

(73) Assignee: Ionis Pharmaceuticals, Inc., Carlsbad, CA (US)

(21) Appl. No.: 17/802,324

(22) PCT Filed: Feb. 26, 2021
(86) PCT No.: PCT/US2021/019960

§ 371 Date: Aug. 25, 2022

Related U.S. Application Data
(60) Provisional application No. 63/085,111, filed on Sep. 29, 2020.
(60) Provisional application No. 62/983,555, filed on Feb. 28, 2020.

(51) Int. Cl. C12N 15/113; C07H 21/00; A61K 31/712; A61K 31/7115; A61K 31/7125

(52) U.S. Cl. CPC C12N 15/1138; C07H 21/00; A61K 31/712; A61K 31/7115; A61K 31/7125; C12N 2310/11; C12N 2310/315; C12N 2310/3341; C12N 2310/3527

(57) ABSTRACT

Provided are compounds, methods, and pharmaceutical compositions for modulating SCN1A RNA and/or protein in a cell or subject. Such compounds, methods, and pharmaceutical compositions are useful to ameliorate at least one symptom of a developmental or epileptic encephalopathic disease, such as Dravet Syndrome. Such symptoms include seizures, sudden unexpected death in epilepsy, status epilepticus, behavioral and developmental delays, movement and balance dysfunctions, orthopedic conditions, motor and cognitive dysfunctions, delayed language and speech issues, visual motor integration dysfunctions, visual perception dysfunctions, executive dysfunctions, growth and nutrition issues, sleeping difficulties, chronic infections, sensory integration disorders, and dysautonomia.

### SEQUENCE LISTING

The present application is being filed along with a Sequence Listing in electronic format. The Sequence Listing is provided as a file entitled BIOL0381WOSEQ_ST25.txt, created on Feb. 24, 2021, which is 624 KB in size. The information in the electronic format of the sequence listing is incorporated herein by reference in its entirety.

### FIELD

Provided are compounds, methods, and pharmaceutical compositions for modulating SCN1A RNA and/or protein in a cell or subject. Such compounds, methods, and pharmaceutical compositions are useful to ameliorate at least one symptom of a developmental or epileptic encephalopathic disease, such as, for example, Dravet Syndrome. Such symptoms include seizures, sudden unexpected death in epilepsy, status epilepticus, behavioral and developmental delays, movement and balance dysfunctions, orthopedic conditions, motor and cognitive dysfunctions, delayed language and speech issues, visual motor integration dysfunctions, visual perception dysfunctions, executive dysfunctions, growth and nutrition issues, sleeping difficulties, chronic infections, sensory integration disorders, and dysautonomia.

### BACKGROUND

The human gene SCN1A encodes human SCN1A protein, the alpha-1 subunit of the voltage-gated sodium channel NaV1.1. Mutations in SCN1A lead to developmental and epileptic encephalopathies (DEEs), including Dravet Syndrome (previously known as Severe Myoclonic Epilepsy of Infancy (SMEI)), one of the most severe childhood forms of epilepsy; other epileptic disorders, including, for example, Genetic Epilepsy with Febrile Seizures Plus (GEFS+) and other febrile seizures, Idiopathic/Generic Generalized Epilepsies (IGE/GGE), Temporal Lobe Epilepsy, Myoclonic Astatic Epilepsy (MAE), Lennox-Gastaut Syndrome, and Migrating Partial Epilepsy of Infancy (MMPSI); and familial hemiplegic migraines, with or without epilepsy (Harkin, L. A., et al., 2007, Brain 130, 843-852; Escayg, A., et al., 2010, Epilepsia 51, 1650-1658; Miller I. O, et al., 2007 Nov 29 [Updated 2019 Apr 18]. In: Adam M P, Ardinger H H, Pagon R A, et al., editors. GeneReviews® [Internet]. Seattle (Wash.): University of Washington, Seattle; 1993-2020. Available from: https://www.ncbi.nlm.nih.gov/books/NBK1318/).

DEEs are associated with SCN1A haploinsufficicncy (Parihar, R., et al., 2013, J. Human Genetics, 58, 573-580). Symptoms associated with DEEs, including Dravet Syndrome, include prolonged seizures (often lasting longer than 10 minutes), frequent seizures (for example, convulsive, myoclonic, absence, focal, obtundation status, and tonic seizures), sudden unexpected death in epilepsy, status epilepticus, behavioral and developmental delays, movement and balance dysfunctions, orthopedic conditions, motor and cognitive dysfunctions (for example, ataxia, tremors, dysarthria, pyramidal, and extrapyramidal signs), delayed language and speech issues, visual motor integration dysfunctions, visual perception dysfunctions, executive dysfunctions, growth and nutrition issues, sleeping difficulties, chronic infections, sensory integration disorders, and dysautonomia. Dravet Syndrome patients experience additional neurodevelopmental delays, leading to severe neurological disability (Guzzetta, F., 2011, Epilepsia 52:S2, 35-38; Anwar et al., 2019, Cureus 11, e5006).

Alternative splicing of SCN1A leads to multiple SCN1A transcript variants (Parihar, R., et al., 2013). Certain transcript variants include a nonsense-mediated decay-inducing exon (NIE) (Steward, C. A., et al., 2019, npj Genom. Med. 4, 31; Carvill et al., 2018, American J. Human Genetics, 103, 1022-1029). One such NIE (NIE-1), which is 64 nucleobases in length and located in SCN1A intron 20, causes degradation of the SCN1A transcript (Carvill et al., 2018).

Currently there remains a need for therapies to treat Dravet Syndrome, GEFS+, and other DEEs. It is therefore an object herein to provide compounds, methods, and pharmaceutical compositions for the treatment of such diseases.

### SUMMARY OF THE INVENTION

Provided herein are compounds, methods, and pharmaceutical compositions for modulating splicing of SCN1A RNA and/or protein in a cell or a subject. In certain embodiments, the amount of SCN1A RNA and/or SCN1A protein is increased. In certain embodiments, the amount of full-length SCN1A RNA and/or full-length SCN1A protein is increased. In certain embodiments, the amount of SCN1A RNA including an NIE is reduced. In certain embodiments, the amount of SCN1A RNA excluding an NIE is increased. In certain embodiments, the NIE is NIE-1. In certain embodiments, the subject has a developmental or epileptic encephalopathy (DEE). In certain embodiments, the DEE is caused by SCN1A haploinsufficiency. In certain embodiments, the DEE is treated by increasing the amount of full-length SCN1A RNA and/or full-length SCN1A protein in a subject, or cell thereof, with compounds capable of excluding an NIE from an SCN1A RNA. In certain embodiments, exclusion of an NIE from an SCN1A RNA increases full-length SCN1A RNA and/or full-length SCN1A protein wherein removal of the NIE prevents degradation of the SCN1A transcript via the NMD pathway. In certain embodiments, the subject has Dravet Syndrome. In certain embodiments, compounds useful for modulating splicing of SCN1A RNA are oligomeric compounds. In certain embodiments, the oligomeric compound comprises or consists of a modified oligonucleotide.

Also provided are methods useful for ameliorating at least one symptom of a DEE. In certain embodiments, the DEE is Dravet Syndrome. In certain embodiments, symptoms include prolonged seizures (often lasting longer than 10 minutes), frequent seizures (for example, convulsive, myoclonic, absence, focal, obtundation status, and tonic seizures), sudden unexpected death in epilepsy, status epilepticus, behavioral and developmental delays, movement and balance dysfunctions, orthopedic conditions, motor and cognitive dysfunctions (for example, ataxia, tremors, dysarthria, pyramidal, and extrapyramidal signs), delayed language and speech issues, visual motor integration dysfunctions, visual perception dysfunctions, executive dysfunctions, growth and nutrition issues, sleeping difficulties, chronic infections, sensory integration disorders, and dysautonomia. In certain embodiments, provided herein are modified oligonucleotides for treating Dravet Syndrome.

### DETAILED DESCRIPTION OF THE INVENTION

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, and GenBank and NCBI reference sequence records are hereby expressly incorporated-by-reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout in the disclosure are incorporated by reference herein in their entirety.

Unless otherwise indicated, the following terms have the following meanings:

As used herein, “2′-deoxyribonucleoside” means a nucleoside comprising a 2′-H(H) deoxyribosyl sugar moiety. In certain embodiments, a 2′-deoxyribonucleoside is a 2′-β-D deoxyribonucleoside and comprises a 2′-β-D-deoxyribosyl sugar moiety, which has the β-D configuration as found in naturally occurring deoxyribonucleic acids (DNA). In certain embodiments, a 2′-deoxyribonucleoside may comprise a modified nucleobase or may comprise an RNA nucleobase (uracil).

As used herein, “2′-MOE” means a 2′-OCH₂CH₂OCH₃ group in place of the 2′—OH group of a ribosyl sugar moiety. A “2′-MOE sugar moiety” is a sugar moiety with a 2′-OCH₂CH₂OCH₃ group in place of the 2′-OH group of a ribosyl sugar moiety. Unless otherwise indicated, a 2′-MOE sugar moiety is in the β-D configuration. “MOE” means O-methoxyethyl.

As used herein, “2′-MOE nucleoside” means a nucleoside comprising a 2′-MOE sugar moiety.

As used herein, “2′-NMA” means a —O—CH₂—C(═O)—NH—CH₃ group in place of the 2′—OH group of a ribosyl sugar moiety. A “2′-NMA sugar moiety” is a sugar moiety with a 2′-O—CH₂—C(═O)—NH—CH₃ group in place of the 2′—OH group of a ribosyl sugar moiety. Unless otherwise indicated, a 2′-NMA sugar moiety is in the β-D configuration. “NMA” means O—N-methyl acetamide.

As used herein, “2′-NMA nucleoside” means a nucleoside comprising a 2′-NMA sugar moiety.

As used herein, “2′-OMe” means a 2′-OCH₃ group in place of the 2′-OH group of a ribosyl sugar moiety. A “2′-OMe sugar moiety” is a sugar moiety with a 2′-OCH₃ group in place of the 2′—OH group of a ribosyl sugar moiety. Unless otherwise indicated, a 2′-OMe sugar moiety is in the β-D configuration. “OMe” means O-methyl.

As used herein, “2′-OMe nucleoside” means a nucleoside comprising a 2′-OMe sugar moiety.

As used herein, “2′-substituted nucleoside” means a nucleoside comprising a 2′-substituted sugar moiety. As used herein, “2′-substituted” in reference to a sugar moiety means a sugar moiety comprising at least one 2′-substituent group other than H or OH.

As used herein, “5-methyl cytosine” means a cytosine modified with a methyl group attached to the 5 position. A 5-methyl cytosine is a modified nucleobase.

As used herein, “administering” means providing a pharmaceutical agent to a subject.

As used herein, “ameliorate” in reference to a treatment means improvement in at least one symptom relative to the same symptom in the absence of the treatment. In certain embodiments, amelioration is the reduction in the severity or frequency of a symptom, or the delayed onset or slowing of progression in the severity or frequency of a symptom. In certain embodiments, the symptom is prolonged seizures (often lasting longer than 10 minutes), frequent seizures (for example, convulsive, myoclonic, absence, focal, obtundation status, and tonic seizures), sudden unexpected death in epilepsy, status epilepticus, behavioral and developmental delays, movement and balance dysfunctions, orthopedic conditions, motor and cognitive dysfunctions (for example, ataxia, tremors, dysarthria, pyramidal, and extrapyramidal signs), delayed language and speech issues, visual motor integration dysfunctions, visual perception dysfunctions, executive dysfunctions, growth and nutrition issues, sleeping difficulties, chronic infections, sensory integration disorders, and dysautonomia.

As used herein, “antisense activity” means any detectable and/or measurable change attributable to the hybridization of an antisense compound to its target nucleic acid.

As used herein, “antisense compound” means an oligomeric compound or oligomeric duplex capable of achieving at least one antisense activity.

As used herein, “bicyclic nucleoside” or “BNA” means a nucleoside comprising a bicyclic sugar moiety.

As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the furanosyl moiety is a ribosyl moiety. In certain embodiments, the bicyclic sugar moiety does not comprise a furanosyl moiety.

As used herein, “cerebrospinal fluid” or “CSF” means the fluid filling the space around the brain and spinal cord. “Artificial cerebrospinal fluid” or “aCSF” means a prepared or manufactured fluid that has certain properties of cerebrospinal fluid.

As used herein, “cEt” means a 4′ to 2′ bridge in place of the 2′OH— group of a ribosyl sugar moiety, wherein the bridge has the formula of 4′-CH(CH₃)—O-2′, and wherein the methyl group of the bridge is in the S configuration. A “cEt sugar moiety” is a bicyclic sugar moiety with a 4′ to 2′ bridge in place of the 2′OH— group of a ribosyl sugar moiety, wherein the bridge has the formula of 4′-CH(CH₃)—O-2′, and wherein the methyl group of the bridge is in the S configuration. “cEt” means constrained ethyl.

As used herein, “cEt nucleoside” means a nucleoside comprising a cEt sugar moiety.

As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more stereorandom chiral centers. In certain embodiments, the molecules are modified oligonucleotides. In certain embodiments, the molecules are compounds comprising modified oligonucleotides.

As used herein, “complementary” in reference to an oligonucleotide means that at least 70% of the nucleobases of the oligonucleotide or one or more portions thereof and the nucleobases of another nucleic acid or one or more portions thereof are capable of hydrogen bonding with one another when the nucleobase sequence of the oligonucleotide and the other nucleic acid are aligned in opposing directions. Complementary nucleobases means nucleobases that are capable of forming hydrogen bonds with one another. Complementary nucleobase pairs include adenine (A) with thymine (T), adenine (A) with uracil (U), cytosine (C) with guanine (G), and 5-methyl cytosine (^{m}C) with guanine (G). Complementary oligonucleotides and/or target nucleic acids need not have nucleobase complementarity at each nucleoside. Rather, some mismatches are tolerated. As used herein, “fully complementary” or “100% complementary” in reference to an oligonucleotide, or a portion thereof, means that the oligonucleotide, or portion thereof, is complementary to another oligonucleotide or target nucleic acid at each nucleobase of the shorter of the two oligonucleotides, or at each nucleoside if the oligonucleotides are the same length.

As used herein, “contiguous” in the context of an oligonucleotide refers to nucleosides, nucleobases, sugar moieties, or intemucleoside linkages that are immediately adjacent to each other. For example, “contiguous nucleobases” means nucleobases that are immediately adjacent to each other in a sequence.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, “internucleoside linkage” means the covalent linkage between contiguous nucleosides in an oligonucleotide. As used herein, “modified internucleoside linkage” means any internucleoside linkage other than a phosphodiester internucleoside linkage. “Phosphorothioate internucleoside linkage” is a modified internucleoside linkage in which one of the non-bridging oxygen atoms of a phosphodiester internucleoside linkage is replaced with a sulfur atom.

As used herein, “mismatch” or “non-complementary” means a nucleobase of a first oligonucleotide that is not complementary with the corresponding nucleobase of a second oligonucleotide or target nucleic acid when the first and second oligonucleotide are aligned.

As used herein, “motif” means the pattern of unmodified and/or modified sugar moieties, nucleobases, and/or internucleoside linkages, in an oligonucleotide.

As used herein, “nonsense-mediated decay-inducing exon (NIE)” is an exon, or a pseudo-exon, that, when included in an mRNA transcript can activate the nonsense-mediated decay (NMD) pathway. “NIE-1” is a 64 nucleobase in length NIE located in intron 20 (chr2:166863579-166864271, hg19; Carvill et al., 2018), which causes degradation of the SCN1A transcript. In certain embodiments, human NIE-1 has the nucleobase sequence of SEQ ID NO: 13. In certain embodiments, mouse NIE-1 has the nucleobase sequence of SEQ ID NO: 14.

As used herein, “non-bicyclic modified sugar moiety” means a modified sugar moiety that comprises a modification, such as a substituent, that does not form a bridge between two atoms of the sugar to form a second ring.

As used herein, “nucleobase” means an unmodified nucleobase or a modified nucleobase. As used herein an “unmodified nucleobase” is adenine (A), thymine (T), cytosine (C), uracil (U), or guanine (G). As used herein, a “modified nucleobase” is a group of atoms other than unmodified A, T, C, U, or G capable of pairing with at least one unmodified nucleobase. A “5-methyl cytosine” is a modified nucleobase. A universal base is a modified nucleobase that can pair with any one of the five unmodified nucleobases. As used herein, “nucleobase sequence” means the order of contiguous nucleobases in a nucleic acid or oligonucleotide independent of any sugar or internucleoside linkage modification.

As used herein, “nucleoside” means a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. “Linked nucleosides” are nucleosides that are connected in a contiguous sequence (i.e., no additional nucleosides are presented between those that are linked).

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound. The term “oligomeric duplex” means a duplex formed by two oligomeric compounds having complementary nucleobase sequences. Each oligomeric compound of an oligomeric duplex may be referred to as a “duplexed oligomenc compound.”

As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or intemucleoside linkage is modified. As used herein, “unmodified oligonucleotide” means an oligonucleotide that does not comprise any nucleoside modifications or intemucleoside modifications.

As used herein, “pharmaceutical composition” means a mixture of substances suitable for administering to a subject. For example, a pharmaceutical composition may comprise an oligomeric compound and a sterile aqueous solution.

As used herein, “pharmaceutically acceptable carrier or diluent” means any substance suitable for use in administering to a subject. Certain such carriers enable pharmaceutical compositions to be formulated as, for example, tablets, pills, dragees, capsules, liquids, gels, syrups, slurries, suspension, and lozenges for the oral ingestion by a subject. In certain embodiments, a pharmaceutically acceptable carrier or diluent is sterile water, sterile saline, sterile buffer solution, or sterile artificial cerebrospinal fluid.

As used herein, “pharmaceutically acceptable salts” means physiologically and pharmaceutically acceptable salts of compounds. Pharmaceutically acceptable salts retain the desired biological activity of the parent compound and do not impart undesired toxicological effects thereto.

As used herein, “RNA” means an RNA transcript and includes pre-mRNA and mature mRNA unless otherwise specified.

As used herein, “stereorandom chiral center” in the context of a population of molecules of identical molecular formula means a chiral center having a random stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center. The stereochemical configuration of a chiral center is considered random when it is the result of a synthetic method that is not designed to control the stereochemical configuration. In certain embodiments, a stereorandom chiral center is a stereorandom phosphorothioate intemucleoside linkage.

As used herein, “subject” means a human or non-human animal.

As used herein, “sugar moiety” means an unmodified sugar moiety or a modified sugar moiety. As used herein, “unmodified sugar moiety” means a 2′-OH(H) β-D ribosyl moiety, as found in RNA (an “unmodified RNA sugar moiety”), or a 2′-H(H) β-D deoxyribosyl moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate.

As used herein, “sugar surrogate” means a modified sugar moiety having other than a furanosyl moiety that can link a nucleobase to another group, such as an intemucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or target nucleic acids.

As used herein, “standard in vitro assay” means the assay described in Example 1 and reasonable variations thereof.

As used herein, “standard in vivo assay” means the assay described in Example 9 and reasonable variations thereof.

As used herein, “symptom” means any physical feature or test result that indicates the existence or extent of a disease or disorder. In certain embodiments, a symptom is apparent to a subject or to a medical professional examining or testing the subject.

As used herein, “target nucleic acid” means a nucleic acid that an antisense compound is designed to affect.

As used herein, “target region” means a portion of a target nucleic acid to which an oligomeric compound is designed to hybridize.

As used herein, “terminal group” means a chemical group or group of atoms that is covalently linked to a terminus of an oligonucleotide.

As used herein, “therapeutically effective amount” means an amount of a pharmaceutical agent that provides a therapeutic benefit to a subject. For example, a therapeutically effective amount improves a symptom of a disease.

### CERTAIN EMBODIMENTS

The present disclosure provides the following non-limiting numbered embodiments:

Embodiment 1. An oligomeric compound comprising a modified oligonucleotide consisting of 12 to 30 linked nucleosides wherein the nucleobase sequence of the modified oligonucleotide is at least 85% complementary to an equal length portion of a SCN1A nucleic acid, and wherein the modified oligonucleotide comprises at least one modification selected from a modified sugar moiety and a modified internucleoside linkage.

Embodiment 2. An oligomeric compound comprising a modified oligonucleotide consisting of 12 to 30 linked nucleosides and having a nucleobase sequence comprising at least 12, at least 13, at least 14, at least 15, at least 16, at least 17, or 18 contiguous nucleobases of any of the nucleobase sequences of SEQ ID NOs 41-889, wherein the modified oligonucleotide comprises at least one modification selected from a modified sugar moiety and a modified intemucleoside linkage.

Embodiment 3. The oligomeric compound of embodiment 1 or embodiment 2, wherein the modified oligonucleotide has a nucleobase sequence that is at least 90%, 95%, or 100% complementary to the nucleobase sequence of SEQ ID NO: 1 or SEQ ID NO: 2 when measured across the entire nucleobase sequence of the modified oligonucleotide.

Embodiment 4. The oligomeric compound of any of embodiments 1-3, wherein the modified oligonucleotide has a nucleobase sequence comprising a portion of at least 8, at least 9, at least 10, at least 11, at least 12, at least 13, at least 14, at least 15, at least 16, at least 17, or at least 18 contiguous nucleobases, wherein the portion is complementary to SEQ ID NO: 13.

Embodiment 5. The oligomeric compound of any of embodiments 1-4 wherein the modified oligonucleotide comprises at least one modified sugar moiety.

Embodiment 6. The oligomeric compound of embodiment 5, wherein the modified oligonucleotide comprises at least one bicyclic sugar moiety.

Embodiment 7. The oligomeric compound of embodiment 6, wherein the bicyclic sugar moiety has a 4′-2′ bridge, wherein the 4′-2′ bridge is selected from —CH₂—O—; and —CH(CH₃)—O—.

Embodiment 8. The oligomeric compound of any of embodiments 5-7, wherein the modified oligonucleotide comprises at least one non-bicyclic modified sugar moiety.

Embodiment 9. The oligomeric compound of embodiment 8, wherein the non-bicyclic modified sugar moiety is any of a 2′-MOE sugar moiety, a 2′-NMA sugar moiety, a 2′-OMe sugar moiety, or a 2′-F sugar moiety.

Embodiment 10. The oligomeric compound any of embodiments 5-9, wherein the modified oligonucleotide comprises at least one sugar surrogate.

Embodiment 11. The oligomeric compound of embodiment 10, wherein the sugar surrogate is any of morpholino, modified morpholino, PNA, THP, and F-HNA.

Embodiment 12. The oligomeric compound of embodiment 5, wherein each nucleoside of the modified oligonucleotide comprises a modified sugar moiety.

Embodiment 13. The oligomeric compound of embodiment 12, wherein each modified sugar moiety is a 2′-MOE sugar moiety.

Embodiment 14. The oligomeric compound of embodiment 12, wherein each modified sugar moiety is a 2′-NMA sugar moiety.

Embodiment 15. The oligomeric compound of any of embodiments 1-14, wherein the modified oligonucleotide comprises at least one modified internucleoside linkage.

Embodiment 16. The oligomeric compound of any of embodiments 1-14, wherein each internucleoside linkage of the modified oligonucleotide is a modified internucleoside linkage.

Embodiment 17. The oligomeric compound of embodiment 15 or embodiment 16, wherein at least one modified internucleoside linkage is a phosphorothioate internucleoside linkage.

Embodiment 18. The oligomeric compound of embodiment 15 or embodiment 17 wherein the modified oligonucleotide comprises at least one phosphodiester internucleoside linkage.

Embodiment 19. The oligomeric compound of any of embodiments 15, 17, or 18, wherein each internucleoside linkage is independently selected from a phosphodiester internucleoside linkage and a phosphorothioate internucleoside linkage.

Embodiment 20. The oligomeric compound of embodiment 16, wherein each internucleoside linkage is a phosphorothioate internucleoside linkage.

Embodiment 21. The oligomeric compound of any of embodiments 1-20, wherein the modified oligonucleotide comprises at least one modified nucleobase.

Embodiment 22. The oligomeric compound of embodiment 21, wherein the modified nucleobase is a 5-methyl cytosine.

Embodiment 23. The oligomeric compound of any of embodiments 1-22, wherein the modified oligonucleotide consists of 12-22, 12-20, 14-20, 15-25, 16-20, 18-22 or 18-20 linked nucleosides.

Embodiment 24. The oligomeric compound of any of embodiments 1-23, wherein the modified oligonucleotide consists of 16, 17, or 18 linked nucleosides.

Embodiment 25. An oligomeric compound comprising a modified oligonucleotide according to the following chemical notation: Aₙₛ Gₙₛ Tₙₛ Tₙₛ Gₙₛ Gₙₛ Aₙₛ Gₙₛ ^{m}Cₙₛ Aₙₛ Aₙₛ Gₙₛ Aₙₛ Tₙₛ Tₙₛ Aₙₛ Tₙₛ ^{m}Cₙ (SEQ ID NO: 41), wherein,
A=an adenine nucleobase,
^{m}C=a 5-methyl cytosine nucleobase,
G=a guanine nucleobase,
T=a thymine nucleobase,
n=a 2′-NMA sugar moiety, and
s=a phosphorothioate internucleoside linkage.

Embodiment 26. An oligomeric compound comprising a modified oligonucleotide according to any one of the following chemical notations (5′ to 3′):

i)
(SEQ ID NO: 41)
AₑₛGₑₒTₑₒTₑₛGₑₛGₑₛAₑₛGₑₛ^{m}CₑₛAₑₛAₑₛGₑₛAₑₛTₑₛTₑₛAₑₛTₑₛ^{m}Cₑ;
ii)
(SEQ ID NO: 41)
AₑₛGₑₒTₑₛTₑₒGₑₛGₑₛAₑₛGₑₛ^{m}CₑₛAₑₛAₑₛGₑₛAₑₛTₑₛTₑₛAₑₛTₑₛ^{m}Cₑ;
iii)
(SEQ ID NO: 41)
AₑₛGₑₒTₑₛTₑₛGₑₛGₑₒAₑₛGₑₛ^{m}CₑₛAₑₛAₑₛGₑₛAₑₛTₑₛTₑₛAₑₛTₑₛ^{m}Cₑ;
iv)
(SEQ ID NO: 41)
AₑₛGₑₒTₑₛTₑₛGₑₛGₑₛAₑₛGₑₒ^{m}CₑₛAₑₛAₑₛGₑₛAₑₛTₑₛTₑₛAₑₛTₑₛ^{m}Cₑ;
v)
(SEQ ID NO: 41)
AₑₛGₑₛTₑₛTₑₒGₑₒGₑₛAₑₛGₑₛ^{m}CₑₛAₑₛAₑₛGₑₛAₑₛTₑₛTₑₛAₑₛTₑₛ^{m}Cₑ;
vi)
(SEQ ID NO: 41)
AₑₛGₑₛTₑₛTₑₛGₑₛGₑₛAₑₛGₑₒ^{m}CₑₒAₑₛAₑₛGₑₛAₑₛTₑₛTₑₛAₑₛTₑₛ^{m}Cₑ;
vii)
(SEQ ID NO: 41)
AₑₛGₑₛTₑₛTₑₛGₑₛGₑₛAₑₛGₑₛ^{m}CₑₛAₑₒAₑₒGₑₛAₑₛTₑₛTₑₛAₑₛTₑₛ^{m}Cₑ;
viii)
(SEQ ID NO: 41)
AₑₛGₑₛTₑₛTₑₛGₑₛGₑₛAₑₛGₑₛ^{m}CₑₛAₑₛAₑₛGₑₒAₑₒTₑₛTₑₛAₑₛTₑₛ^{m}Cₑ;
ix)
(SEQ ID NO: 41)
AₑₛGₑₒTₑₛTₑₛGₑₛGₑₛAₑₛGₑₛ^{m}CₑₛAₑₒAₑₛGₑₛAₑₛTₑₛTₑₛAₑₛTₑₛ^{m}Cₑ;
x)
(SEQ ID NO: 41)
AₑₛGₑₒTₑₛTₑₛGₑₛGₑₛAₑₛGₑₛ^{m}CₑₛAₑₛAₑₛGₑₒAₑₛTₑₛTₑₛAₑₛTₑₛ^{m}Cₑ;
xi)
(SEQ ID NO: 41)
AₑₛGₑₒTₑₛTₑₛGₑₛGₑₛAₑₛGₑₛ^{m}CₑₛAₑₛAₑₛGₑₛAₑₛTₑₛTₑₒAₑₛTₑₛ^{m}Cₑ;
xii)
(SEQ ID NO: 41)
AₑₛGₑₛTₑₒTₑₛGₑₛGₑₛAₑₛGₑₛ^{m}CₑₛAₑₛAₑₛGₑₛAₑₛTₑₛTₑₒAₑₛTₑₛ^{m}Cₑ;
xiii)
(SEQ ID NO: 41)
AₑₛGₑₛTₑₛTₑₛGₑₒGₑₛAₑₛGₑₛ^{m}CₑₛAₑₛAₑₛGₑₛAₑₛTₑₛTₑₒAₑₛTₑₛ^{m}Cₑ;
xiv)
(SEQ ID NO: 41)
AₑₛGₑₛTₑₛTₑₛGₑₛGₑₒAₑₒGₑₛ^{m}CₑₛAₑₛAₑₛGₑₛAₑₛTₑₛTₑₛAₑₛTₑₛ^{m}Cₑ;
xv)
(SEQ ID NO: 41)
AₑₛGₑₛTₑₛTₑₛGₑₛGₑₛAₑₒGₑₛ^{m}CₑₛAₑₛAₑₛGₑₛAₑₛTₑₛTₑₒAₑₛTₑₛ^{m}Cₑ;
xvi)
(SEQ ID NO: 41)
AₑₛGₑₛTₑₛTₑₛGₑₛGₑₛAₑₛGₑₛ^{m}CₑₒAₑₛAₑₛGₑₛAₑₛTₑₒTₑₛAₑₛTₑₛ^{m}Cₑ;
xvii)
(SEQ ID NO: 41)
AₑₛGₑₛTₑₛTₑₛGₑₛGₑₛAₑₛGₑₛ^{m}CₑₒAₑₛAₑₛGₑₛAₑₛTₑₛTₑₒAₑₛTₑₛ^{m}Cₑ;
xviii)
(SEQ ID NO: 41)
AₑₛGₑₛTₑₛTₑₛGₑₛGₑₛAₑₛGₑₛ^{m}CₑₛAₑₛAₑₒGₑₛAₑₛTₑₛTₑₒAₑₒTₑₛ^{m}Cₑ;
xix)
(SEQ ID NO: 41)
AₑₛGₑₛTₑₛTₑₛGₑₛGₑₛAₑₛGₑₛ^{m}CₑₛAₑₛAₑₛGₑₒAₑₛTₑₒTₑₛAₑₛTₑₛ^{m}Cₑ;
xx)
(SEQ ID NO: 41)
AₑₛGₑₛTₑₛTₑₛGₑₛGₑₛAₑₛGₑₛ^{m}CₑₛAₑₛAₑₛGₑₛAₑₒTₑₛTₑₒAₑₛTₑₛ^{m}Cₑ;
xxi)
(SEQ ID NO: 41)
AₑₛGₑₛTₑₛTₑₛGₑₛGₑₛAₑₛGₑₛ^{m}CₑₛAₑₛAₑₛGₑₛAₑₛTₑₒTₑₒAₑₛTₑₛ^{m}Cₑ;
xxii)
(SEQ ID NO: 890)
AₑₒAₑₛGₑₛTₑₛTₑₛGₑₛGₑₛAₑₛGₑₛ^{m}CₑₛAₑₛAₑₛGₑₛAₑₛTₑₛTₑₛAₑₛTₑₛ^{m}Cₑₒ^{m}Cₑ;
xxiii)
(SEQ ID NO: 891)
AₑₛGₑₛTₑₛTₑₛGₑₛGₑₛAₑₛGₑₛ^{m}CₑₛAₑₛAₑₛGₑₛAₑₛTₑₛTₑₛAₑₛTₑₛ^{m}Cₑₒ^{m}Cₑ;
xxiv)
(SEQ ID NO: 892)
AₑₒAₑₛGₑₛTₑₛTₑₛGₑₛGₑₛAₑₛGₑₛ^{m}CₑₛAₑₛAₑₛGₑₛAₑₛTₑₛTₑₛAₑₛTₑₛ^{m}Cₑ;
xxv)
(SEQ ID NO: 881)
GₙₛGₙₛTₙₛAₙₛGₙₛ^{m}CₙₛAₙₛAₙₛAₙₛAₙₛGₙₛGₙₛGₙₛGₙₛTₙₛAₙₛAₙₛTₙ;
xxvi)
(SEQ ID NO: 885)
AₙₛGₙₛ^{m}CₙₛAₙₛAₙₛAₙₛAₙₛGₙₛGₙₛGₙₛGₙₛTₙₛAₙₛAₙₛTₙₛAₙₛ^{m}CₙₛAₙ;
xxvii)
(SEQ ID NO: 888)
Gₙₛ^{m}CₙₛAₙₛAₙₛAₙₛAₙₛGₙₛGₙₛGₙₛGₙₛTₙₛAₙₛAₙₛTₙₛAₙₛ^{m}CₙₛAₙₛGₙ;
xxviii)
(SEQ ID NO: 882)
^{m}CₙₛAₙₛAₙₛAₙₛAₙₛGₙₛGₙₛGₙₛGₙₛTₙₛAₙₛAₙₛTₙₛAₙₛ^{m}CₙₛAₙₛGₙₛTₙ;
xxix)
(SEQ ID NO: 880)
AₙₛAₙₛAₙₛGₙₛGₙₛGₙₛGₙₛTₙₛAₙₛAₙₛTₙₛAₙₛ^{m}CₙₛAₙₛGₙₛTₙₛAₙₛ^{m}Cₙ;
xxx)
(SEQ ID NO: 883)
AₙₛAₙₛGₙₛGₙₛGₙₛGₙₛTₙₛAₙₛAₙₛTₙₛAₙₛ^{m}CₙₛAₙₛGₙₛTₙₛAₙₛ^{m}Cₙₛ^{m}Cₙ;
xxxi)
(SEQ ID NO: 511)
GₙₛTₙₛAₙₛAₙₛTₙₛAₙₛ^{m}CₙₛAₙₛGₙₛTₙₛAₙₛ^{m}Cₙₛ^{m}Cₙₛ^{m}CₙₛAₙₛTₙₛAₙₛAₙ;
xxxii)
(SEQ ID NO: 750)
TₙₛAₙₛAₙₛTₙₛAₙₛ^{m}CₙₛAₙₛGₙₛTₙₛAₙₛ^{m}Cₙₛ^{m}Cₙₛ^{m}CₙₛAₙₛTₙₛAₙₛAₙₛTₙ;
xxxiii)
(SEQ ID NO: 736)
AₙₛAₙₛTₙₛAₙₛ^{m}CₙₛAₙₛGₙₛTₙₛAₙₛ^{m}Cₙₛ^{m}Cₙₛ^{m}CₙₛAₙₛTₙₛAₙₛAₙₛTₙₛAₙ;
xxxiv)
(SEQ ID NO: 642)
AₙₛTₙₛAₙₛ^{m}CₙₛAₙₛGₙₛTₙₛAₙₛ^{m}Cₙₛ^{m}Cₙₛ^{m}CₙₛAₙₛTₙₛAₙₛAₙₛTₙₛAₙₛAₙ;
xxxv)
(SEQ ID NO: 519)
TₙₛAₙₛ^{m}CₙₛAₙₛGₙₛTₙₛAₙₛ^{m}Cₙₛ^{m}Cₙₛ^{m}CₙₛAₙₛTₙₛAₙₛAₙₛTₙₛAₙₛAₙₛAₙ;
xxxvi)
(SEQ ID NO: 741)
Aₙₛ^{m}CₙₛAₙₛGₙₛTₙₛAₙₛ^{m}Cₙₛ^{m}Cₙₛ^{m}CₙₛAₙₛTₙₛAₙₛAₙₛTₙₛAₙₛAₙₛAₙₛGₙ;
xxxvii)
(SEQ ID NO: 664)
^{m}Cₙₛ^{m}Cₙₛ^{m}CₙₛAₙₛTₙₛ^{m}Cₙₛ^{m}CₙₛAₙₛAₙₛGₙₛTₙₛTₙₛGₙₛGₙₛAₙₛGₙₛ^{m}CₙₛAₙ;
xxxviii)
(SEQ ID NO: 645)
^{m}Cₙₛ^{m}CₙₛAₙₛTₙₛ^{m}Cₙₛ^{m}CₙₛAₙₛAₙₛGₙₛTₙₛTₙₛGₙₛGₙₛAₙₛGₙₛ^{m}CₙₛAₙₛAₙ;
xxxix)
(SEQ ID NO: 550)
^{m}CₙₛAₙₛTₙₛ^{m}Cₙₛ^{m}CₙₛAₙₛAₙₛGₙₛTₙₛTₙₛGₙₛGₙₛAₙₛGₙₛ^{m}CₙₛAₙₛAₙₛGₙ;
xl)
(SEQ ID NO: 814)
AₙₛTₙₛ^{m}Cₙₛ^{m}CₙₛAₙₛAₙₛGₙₛTₙₛTₙₛGₙₛGₙₛAₙₛGₙₛ^{m}CₙₛAₙₛAₙₛGₙₛAₙ;
xli)
(SEQ ID NO: 692)
Tₙₛ^{m}Cₙₛ^{m}CₙₛAₙₛAₙₛGₙₛTₙₛTₙₛGₙₛGₙₛAₙₛGₙₛ^{m}CₙₛAₙₛAₙₛGₙₛAₙₛTₙ;
xlii)
(SEQ ID NO: 587)
^{m}Cₙₛ^{m}CₙₛAₙₛAₙₛGₙₛTₙₛTₙₛGₙₛGₙₛAₙₛGₙₛ^{m}CₙₛAₙₛAₙₛGₙₛAₙₛTₙₛTₙ;
xliii)
(SEQ ID NO: 523)
^{m}CₙₛAₙₛAₙₛGₙₛTₙₛTₙₛGₙₛGₙₛAₙₛGₙₛ^{m}CₙₛAₙₛAₙₛGₙₛAₙₛTₙₛTₙₛAₙ;
xliv)
(SEQ ID NO: 778)
AₙₛAₙₛGₙₛTₙₛTₙₛGₙₛGₙₛAₙₛGₙₛ^{m}CₙₛAₙₛAₙₛGₙₛAₙₛTₙₛTₙₛAₙₛTₙ;
xlv)
(SEQ ID NO: 510)
TₙₛTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙ;
xlvi)
(SEQ ID NO: 816)
TₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙ;
xlvii)
(SEQ ID NO: 696)
GₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙₛTₙ;
or
xlviii)
(SEQ ID NO: 590)
GₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙₛTₙₛAn,

wherein,
A=an adenine nucleobase,
^{m}C=a 5-methyl cytosine nucleobase,
G=a guanine nucleobase,
T=a thymine nucleobase,
e=a 2′-MOE sugar moiety,
n=a 2′-NMA sugar moiety.
o=a phosphodiester intemucleoside linkage, and
s=a phosphorothioate intemucleoside linkage.

Embodiment 27. The oligomeric compound of any of embodiments 1-26, wherein the oligomeric compound is a singled-stranded oligomeric compound.

Embodiment 28. The oligomeric compound of any of embodiments 1-27 consisting of the modified oligonucleotide.

Embodiment 29. The oligomeric compound of any of embodiments 1-28 comprising a conjugate moiety comprising a conjugate group and a conjugate linker.

Embodiment 30. The oligomeric compound of embodiment 29, wherein the conjugate group comprises a GalNAc cluster comprising 1-3 GalNAc ligands.

Embodiment 31. The oligomeric compound of embodiment 29 or embodiment 30, wherein the conjugate linker consists of a single bond.

Embodiment 32. The oligomeric compound of embodiment 29, wherein the conjugate linker is cleavable.

Embodiment 33. The oligomeric compound of embodiment 29, wherein the conjugate linker comprises 1-3 linker-nucleosides.

Embodiment 34. The oligomeric compound of any of embodiments 29-33, wherein the conjugate group is attached to the modified oligonucleotide at the 5′-end of the modified oligonucleotide.

Embodiment 35. The oligomeric compound of any of embodiments 29-33, wherein the conjugate group is attached to the modified oligonucleotide at the 3′-end of the modified oligonucleotide.

Embodiment 36. The oligomeric compound of any of embodiments 1-27 or 29-35 comprising a terminal group.

Embodiment 37. The oligomeric compound of any of embodiments 1-32 or 34-35, wherein the oligomeric compound does not comprise linker-nucleosides.

Embodiment 38. A modified oligonucleotide according to the following chemical structure:

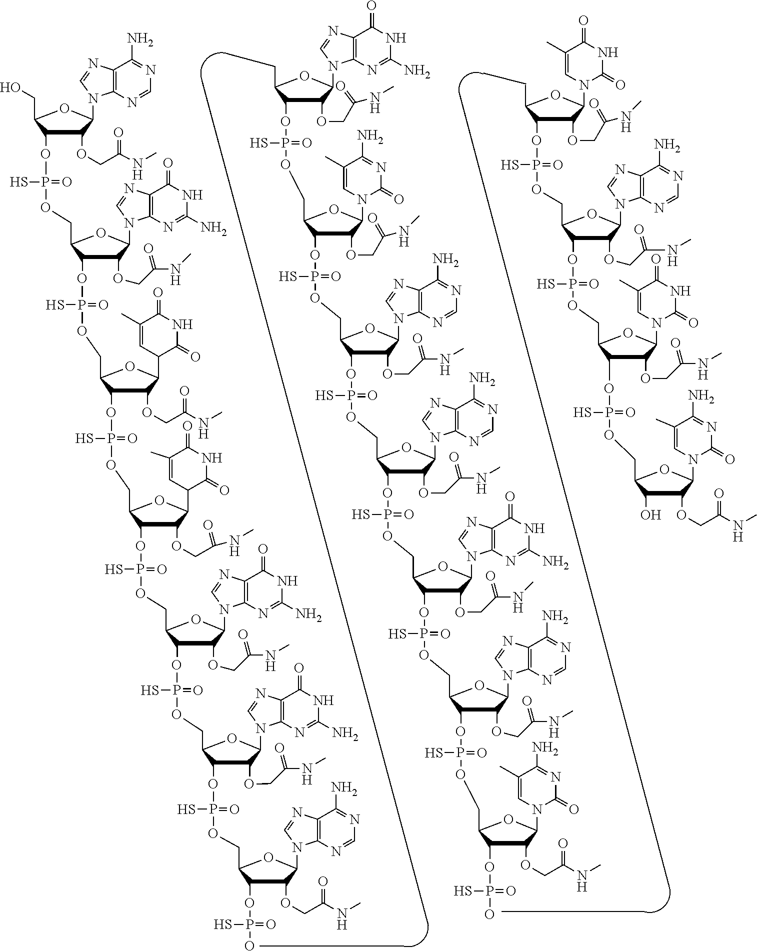

(SEQ ID NO: 41) or a salt thereof.

Embodiment 39. The modified oligonucleotide of embodiment 38, which is the sodium salt or the potassium salt.

Embodiment 40. A modified oligonucleotide according to the following chemical structure:

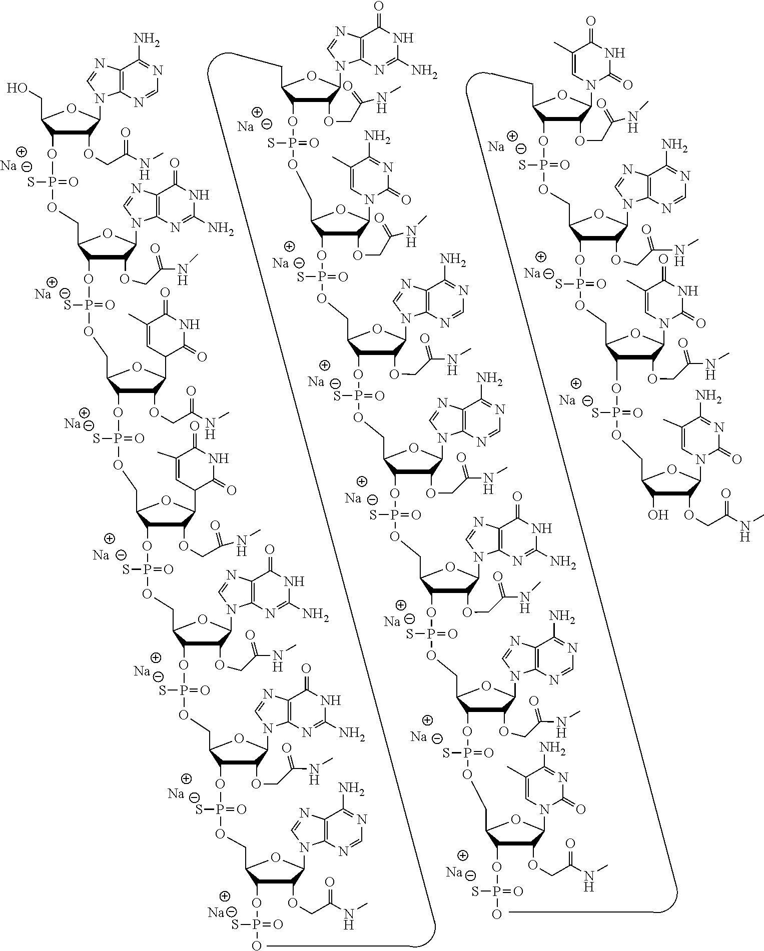

### (SEQ ID NO: 41).

Embodiment 41. A chirally enriched population of modified oligonucleotides of any of embodiments 38-40, wherein the population is enriched for modified oligonucleotides comprising at least one particular phosphorothioate internucleoside linkage having a particular stereochemical configuration.

Embodiment 42. The chirally enriched population of embodiment 41, wherein the population is enriched for modified oligonucleotides comprising at least one particular phosphorothioate internucleoside linkage having the (Sp) configuration.

Embodiment 43. The chirally enriched population of embodiment 41, wherein the population is enriched for modified oligonucleotides comprising at least one particular phosphorothioate internucleoside linkage having the (Rp) configuration.

Embodiment 44. The chirally enriched population of embodiment 41, wherein the population is enriched for modified oligonucleotides having a particular, independently selected stereochemical configuration at each phosphorothioate intemucleoside linkage.

Embodiment 45. The chirally enriched population of embodiment 44, wherein the population is enriched for modified oligonucleotides having the (Sp) configuration at each phosphorothioate internucleoside linkage or for modified oligonucleotides having the (Rp) configuration at each phosphorothioate intemucleoside linkage.

Embodiment 46. The chirally enriched population of embodiment 44, wherein the population is enriched for modified oligonucleotides having the (Rp) configuration at one particular phosphorothioate intemucleoside linkage and the (Sp) configuration at each of the remaining phosphorothioate intemucleoside linkages.

Embodiment 47. The chirally enriched population of embodiment 44, wherein the population is enriched for modified oligonucleotides having at least 3 contiguous phosphorothioate internucleoside linkages in the Sp, Sp, and Rp configurations, in the 5′ to 3′ direction.

Embodiment 48. A population of modified oligonucleotides of any of embodiments 38-40, wherein all of the phosphorothioate intemucleoside linkages of the modified oligonucleotide are stereorandom.

Embodiment 49. A pharmaceutical composition comprising the oligomeric compound of any of embodiments 1-37, the modified oligonucleotide of any of embodiments 38-40, the chirally-enriched population of any of embodiments 41-47, or the population of modified oligonucleotides of embodiment 48, and a pharmaceutically acceptable diluent or carrier.

Embodiment 50. The pharmaceutical composition of embodiment 49, comprising a pharmaceutically acceptable diluent and wherein the pharmaceutically acceptable diluent is artificial CSF (aCSF) or PBS.

Embodiment 51. The pharmaceutical composition of embodiment 50, wherein the pharmaceutical composition consists essentially of the modified oligonucleotide and artificial CSF (aCSF).

Embodiment 52. The pharmaceutical composition of embodiment 50, wherein the pharmaceutical composition consists essentially of the modified oligonucleotide and PBS.

Embodiment 53. A method of modulating splicing of an SCN1A RNA in a cell comprising contacting the cell with an oligomeric compound of any of embodiments 1-37, a modified oligonucleotide of any of embodiments 38-40, a chirally-enriched population of any of embodiments 41-47, a population of modified oligonucleotides of embodiment 48, or a pharmaceutical composition of any of embodiments 49-52.

Embodiment 54. The method of embodiment 53, wherein the amount of SCN1A RNA that includes an NIE is reduced.

Embodiment 55. The method of embodiment 54, wherein the amount of SCN1A RNA that includes NIE-1 is reduced.

Embodiment 56. The method of any of embodiments 53-55, wherein the amount of SCN1A RNA that excludes an NIE is increased.

Embodiment 57. The method of any of embodiments 53-56, wherein the amount of SCN1A RNA that excludes NIE-1 is increased.

Embodiment 58. A method of increasing the amount of full-length SCN1A RNA in a cell, comprising contacting the cell with an oligomeric compound of any of embodiments 1-37, a modified oligonucleotide of any of embodiments 38-40, a chirally-enriched population of any of embodiments 41-47, a population of modified oligonucleotides of embodiment 48, or a pharmaceutical composition of any of embodiments 49-52.

Embodiment 59. A method of increasing SCN1A RNA lacking NIE-1 in a cell, tissue, or organ, comprising contacting a cell, tissue, or organ with an oligomeric compound of any of embodiments 1-37, a modified oligonucleotide of any of embodiments 38-40, a chirally-enriched population of any of embodiments 41-47, a population of modified oligonucleotides of embodiment 48, or a pharmaceutical composition of any of embodiments 49-52.

Embodiment 60. The method of any of embodiments 53-59, wherein the cell is in vitro.

Embodiment 61. The method of any of embodiments 53-59, wherein the cell is in an animal.

Embodiment 62. A method of ameliorating a disease associated with SCN1A comprising administering to a subject having or at risk for developing a disease associated with SCN1A a therapeutically effective amount of a pharmaceutical composition according to any of embodiments 49-52, and thereby treating the disease associated with SCN1A.

Embodiment 63. The method of embodiment 62, comprising identifying a subject having or at risk for developing a disease associated SCN1A.

Embodiment 64. The method of embodiment 62 or embodiment 63, wherein the disease associated with SCN1A is a developmental or epileptic encephalopathic disease.

Embodiment 65. The method of embodiment 64, wherein the developmental or epileptic encephalopathic disease is Dravet Syndrome.

Embodiment 66. The method of embodiment 64, wherein the developmental or epileptic encephalopathic disease is any of Genetic Epilepsy with Febrile Seizures Plus (GEFS+), febrile seizures, Idiopathic/Generic Generalized Epilepsies (IGE/GGE), Temporal Lobe Epilepsy, Myoclonic Astatic Epilepsy (MAE), Lennox-Gastaut Syndrome, or Migrating Partial Epilepsy of Infancy (MMPSI).

Embodiment 67. The method of any of embodiments 64-66, wherein at least one symptom of the developmental or epileptic encephalopathic disease is ameliorated.

Embodiment 68. The method of embodiment 67, wherein the symptom is any of seizures, behavioral and developmental delays, movement and balance dysfunctions, motor and cognitive dysfunctions, delayed language and speech, visual motor integration dysfunctions, visual perception dysfunctions, executive dysfunctions, growth and nutrition issues, sleeping difficulties, chronic infections, sensory integration disorders, or dysautonomia.

Embodiment 69. The method of embodiment 68, wherein the seizures are frequent or prolonged.

Embodiment 70. The method of embodiment 68 or embodiment 69, wherein the seizure is any of convulsive, myoclonic, absence, focal, obtundation status, or tonic.

Embodiment 71. The method of any of embodiments 62-70, wherein the pharmaceutical composition is administered to the central nervous system or systemically.

Embodiment 72. The method of embodiment 71, wherein the pharmaceutical composition is administered to the central nervous system and systemically.

Embodiment 73. The method of any of embodiments 62-71, wherein the pharmaceutical composition is administered any of intrathecally, systemically, subcutaneously, or intramuscularly.

### I. Certain Oligonucleotides

In certain embodiments, provided herein are oligomeric compounds comprising oligonucleotides, which consist of linked nucleosides. Oligonucleotides may be unmodified oligonucleotides (RNA or DNA) or may be modified oligonucleotides. Modified oligonucleotides comprise at least one modification relative to unmodified RNA or DNA. That is, modified oligonucleotides comprise at least one modified nucleoside (comprising a modified sugar moiety and/or a modified nucleobase) and/or at least one modified internucleoside linkage.

A. Certain Modified Nucleosides

Modified nucleosides comprise a modified sugar moiety or a modified nucleobase or both a modified sugar moiety and a modified nucleobase.

1. Certain Sugar Moieties

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified sugar moieties comprising a furanosyl ring with one or more substituent groups none of which bridges two atoms of the furanosyl ring to form a bicyclic structure. Such non bridging substituents may be at any position of the furanosyl, including but not limited to substituents at the 2′, 4′, and/or 5′ positions. In certain embodiments one or more non-bridging substituent of non-bicyclic modified sugar moieties is branched. Examples of 2′-substituent groups suitable for non-bicyclic modified sugar moieties include but are not limited to: 2′-F, 2′-OCH₃(“OMe” or “O-methyl”), and 2′-O(CH₂)₂OCH₃(“MOE” or “O-methoxyethyl”), and 2′-O—N-alkyl acetamide, e.g., 2′-O—N-methyl acetamide (“NMA”), 2′-O—N-dimethyl acetamide, 2′-O—N-ethyl acetamide, or 2′-O—N-propyl acetamide. For example, see U.S. Pat. No. 6,147,200, Prakash et al., 2003, Org. Lett., 5, 403-6. A “2′-O—N-methyl acetamide nucleoside” or “2′-NMA nucleoside” is shown below:

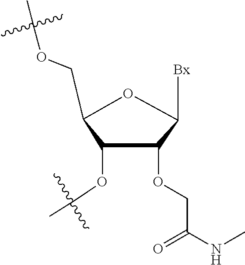

In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, 5-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or 5), 5′-vinyl, and 5′-methoxy. In certain embodiments, non-bicyclic modified sugar moieties comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836.

In certain embodiments, a 2′-substituted non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂), ON(CH₃)₂, O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl, e.g., for example, OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, OCH₃, OCH₂CH₂OCH₃, and OCH2C(═O)—N(H)CH3.

Certain modified sugar moieties comprise a substituent that bridges two atoms of the furanosyl ring to form a second ring, resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt”), 4′-CH₂-0-CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2-, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ-, and —N(Rₐ)—;

wherein:

x is 0, 1, or 2;

n is 1, 2, 3, or 4;

each Rₐ and R_{b} is, independently, H, a protecting group, hydroxyl, C₁-C₁ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and

each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129, 8362-8379; Wengel et a., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

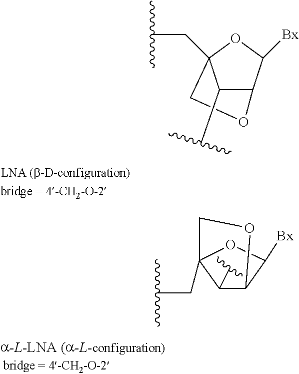

α-L-methyleneoxy (4′-CH₂—O-2′) or α-L-LNA bicyclic nucleosides have been incorporated into oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, C J. Bioorg. & Med. Chem. 2002, 10, 841-854), fluoro HNA:

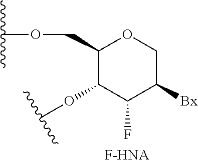

(“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds having the formula:

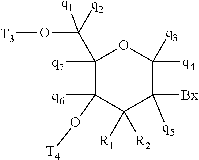

wherein, independently, for each of said modified THP nucleoside:

Bx is a nucleobase moiety;

T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group; q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and

each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

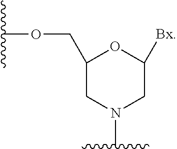

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides.

2. Certain Modified Nucleobases

In certain embodiments, modified oligonucleotides comprise one or more nucleosides comprising an unmodified nucleobase. In certain embodiments, modified oligonucleotides comprise one or more nucleosides comprising a modified nucleobase. In certain embodiments, modified oligonucleotides comprise one or more nucleosides that does not comprise a nucleobase, referred to as an abasic nucleoside.

In certain embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyl adenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

3. Certain Modified Internucleoside Linkages

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include but are not limited to phosphodiesters, which contain a phosphodiester bond, P(O₂)═O, (also referred to as unmodified or naturally occurring linkages); phosphotriesters; methylphosphonates; methoxypropylphosphonates (“MOP”); phosphoramidates; mesyl phosphoramidates; phosphorothioates (P(O₂)═S); and phosphorodithioates (HS—P═S). Representative non-phosphorus containing internucleoside linking groups include but are not limited to methylenemethylimino (—CH₂—N(CH₃)—O—CH₂—); thiodiester, thionocarbamate (—O—C(═O)(NH)—S—); siloxane (—O—SiH₂—O—); and N,N′-dimethylhydrazine (—CH₂—N(CH₃)—N(CH₃)—). Modified intemucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Methods of preparation of phosphorous-containing and non-phosphorous-containing internucleoside linkages are well known to those skilled in the art.

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate internucleoside linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate internucleoside linkage. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate internucleoside linkage is present in at least 65% of the molecules in the population. In certain embodiments, the particular configuration of the particular phosphorothioate intemucleoside linkage is present in at least 70% of the molecules in the population. In certain embodiments, the particular configuration of the particular phosphorothioate internucleoside linkage is present in at least 80% of the molecules in the population. In certain embodiments, the particular configuration of the particular phosphorothioate internucleoside linkage is present in at least 90% of the molecules in the population. In certain embodiments, the particular configuration of the particular phosphorothioate intemucleoside linkage is present in at least 99% of the molecules in the population. Such chirally enriched populations of modified oligonucleotides can be generated using synthetic methods known in the art, e.g., methods described in Oka et al., JACS, 2003, 125, 8307, Wan et al. Nuc. Acid. Res., 2014, 42, 13456, and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

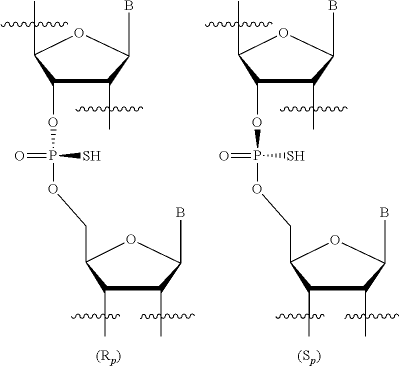

Unless otherwise indicated, chiral internucleoside linkages of modified oligonucleotides described herein can be stereorandom or in a particular stereochemical configuration.

In certain embodiments, modified oligonucleotides comprise an internucleoside motif of (5′ to 3′) sooosssssssssssssss. In certain embodiments, the particular stereochemical configuration of the modified oligonucleotides is (5′ to 3′) Sp-o-o-o-Sp-Sp-Sp-Rp-Sp-Sp-Rp-Sp-Sp-Sp-Sp-Sp-Sp-Sp-Sp or Sp-o-o-o-Sp-Sp-Sp-Rp-Sp-Sp-Sp-Sp-Sp-Sp-Sp-Sp-Sp-Sp-Sp; wherein each ‘Sp’ represents a phosphorothioate internucleoside linkage in the S configuration; Rp represents a phosphorothioate intemucleoside linkage in the R configuration; and ‘o’ represents a phosphodiester intemucleoside linkage.

Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl, and thioformacetal (3′-S—CH₂—O-5′). Further neutral intemucleoside linkages include nonionic linkages comprising siloxane (dialkylsiloxane), carboxylate ester, carboxamide, sulfide, sulfonate ester and amides (see e.g., Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts. In certain embodiments, a modified internucleoside linkage is any of those described in WO 2021/030778, incorporated by reference herein.

B. Certain Motifs

In certain embodiments, modified oligonucleotides comprise one or more modified nucleosides comprising a modified sugar moiety. In certain embodiments, modified oligonucleotides comprise one or more modified nucleosides comprising a modified nucleobase. In certain embodiments, modified oligonucleotides comprise one or more modified internucleoside linkages. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

1. Certain Sugar Motifs

In certain embodiments, oligonucleotides comprise one or more type of modified sugar and/or unmodified sugar moiety arranged along the oligonucleotide, or portion thereof, in a defined pattern or sugar motif. In certain instances, such sugar motifs include but are not limited to any of the sugar modifications discussed herein.

In certain embodiments, modified oligonucleotides have a gapmer motif, which is defined by two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction), In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5-wing differs from the sugar motif of the 3-wing (asymmetric gapmer).

In certain embodiments, the wings of a gapmer comprise 1-6 nucleosides. In certain embodiments, each nucleoside of each wing of a gapmer comprises a modified sugar moiety. In certain embodiments, at least one, at least two, at least three, at least four, at least five, or at least six nucleosides of each wing of a gapmer comprises a modified sugar moiety.

In certain embodiments, the gap of a gapmer comprises 7-12 nucleosides. In certain embodiments, each nucleoside of the gap of a gapmer comprises a 2′-deoxyribosyl sugar moiety. In certain embodiments, at least one nucleoside of the gap of a gapmer comprises a modified sugar moiety and each remaining nucleoside comprises a 2′-deoxyribosyl sugar moiety.

Herein, the lengths (number of nucleosides) of the three regions of a gapmer may be provided using the notation [#of nucleosides in the 5′-wing]-[#of nucleosides in the gap]-[#of nucleosides in the 3′-wing]. Thus, a 5-10-5 gapmer consists of 5 linked nucleosides in each wing and 10 linked nucleosides in the gap. Where such nomenclature is followed by a specific modification, that modification is the modification in each sugar moiety of each wing and the gap nucleosides comprise a 2′-deoxyribosyl sugar moiety. Thus, a 5-10-5 MOE gapmer consists of 5 linked 2′-MOE nucleosides in the 5′-wing, 10 linked 2′-deoxyribonucleosides in the gap, and 5 linked 2′-MOE nucleosides in the 3′-wing.

In certain embodiments, each nucleoside of a modified oligonucleotide, or portion thereof, comprises a 2′-substituted sugar moiety, a bicyclic sugar moiety, a sugar surrogate, or a 2′-deoxyribosyl sugar moiety. In certain embodiments, the 2′-substituted sugar moiety is selected from a 2′-MOE sugar moiety, a 2′-NMA sugar moiety, a 2′-OMe sugar moiety, and a 2′-F sugar moiety. In certain embodiments, the bicyclic sugar moiety is selected from a cEt sugar moiety and an LNA sugar moiety. In certain embodiments, the sugar surrogate is selected from morpholino, modified morpholino, PNA, THP, and F-HNA.

In certain embodiments, modified oligonucleotides comprise at least 12, at least 13, at least 14, at least 15, at least 16, at least 17, at least 18, at least 19, or at least 20 nucleosides comprising a modified sugar moiety. In certain embodiments, the modified sugar moiety is selected independently from a 2′-substituted sugar moiety, a bicyclic sugar moiety, or a sugar surrogate. In certain embodiments, the 2′-substituted sugar moiety is selected from a 2′-MOE sugar moiety, a 2′-NMA sugar moiety, a 2′-OMe sugar moiety, and a 2′-F sugar moiety. In certain embodiments, the bicyclic sugar moiety is selected from a cEt sugar moiety and an LNA sugar moiety. In certain embodiments, the sugar surrogate is selected from morpholino, modified morpholino, THP, and F-HNA.

In certain embodiments, each nucleoside of a modified oligonucleotide comprises a modified sugar moiety (“fully modified oligonucleotide”). In certain embodiments, each nucleoside of a fully modified oligonucleotide comprises a 2′-substituted sugar moiety, a bicyclic sugar moiety, or a sugar surrogate. In certain embodiments, the 2′-substituted sugar moiety is selected from a 2′-MOE sugar moiety, a 2′-NMA sugar moiety, a 2′-OMe sugar moiety, and a 2′-F sugar moiety. In certain embodiments, the bicyclic sugar moiety is selected from a cEt sugar moiety and an LNA sugar moiety. In certain embodiments, the sugar surrogate is selected from morpholino, modified morpholino, THP, and F-HNA. In certain embodiments, each nucleoside of a fully modified oligonucleotide comprises the same modified sugar moiety (“uniformly modified sugar motif”). In certain embodiments, the uniformly modified sugar motif is 7 to 20 nucleosides in length. In certain embodiments, each nucleoside of the uniformly modified sugar motif comprises a 2′-substituted sugar moiety, a bicyclic sugar moiety, or a sugar surrogate. In certain embodiments, the 2′-substituted sugar moiety is selected from a 2′-MOE sugar moiety, a 2′-NMA sugar moiety, a 2′-OMe sugar moiety, and a 2′-F sugar moiety. In certain embodiments, the bicyclic sugar moiety is selected from a cEt sugar moiety and an LNA sugar moiety. In certain embodiments, the sugar surrogate is selected from morpholino, modified morpholino, THP, and F-HNA. In certain embodiments, modified oligonucleotides having at least one fully modified sugar motif may also comprise at least 1, at least 2, at least 3, or at least 4 2′-deoxyribonucleosides.

2. Certain Nucleobase Motifs

In certain embodiments, oligonucleotides comprise modified and/or unmodified nucleobases arranged along the oligonucleotide, or portion thereof, in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methyl cytosines. In certain embodiments, all of the cytosine nucleobases are 5-methyl cytosines and all of the other nucleobases of the modified oligonucleotide are unmodified nucleobases.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of the nucleoside is a 2′-deoxyribosyl sugar moiety. In certain embodiments, the modified nucleobase is selected from: a 2-thiopyrimidine and a 5-propynepyrimidine.

3. Certain Internucleoside Linkage Motifs

In certain embodiments, oligonucleotides comprise modified and/or unmodified intemucleoside linkages arranged along the oligonucleotide, or portion thereof, in a defined pattern or motif. In certain embodiments, each intemucleoside linking group is a phosphodiester intemucleoside linkage. In certain embodiments, each intemucleoside linking group of a modified oligonucleotide is a phosphorothioate intemucleoside linkage. In certain embodiments, each intemucleoside linkage of a modified oligonucleotide is independently selected from a phosphorothioate intemucleoside linkage and phosphodiester internucleoside linkage. In certain embodiments, each phosphorothioate intemucleoside linkage is independently selected from a stereorandom phosphorothioate, a (Sp) phosphorothioate, and a (Rp) phosphorothioate. In certain embodiments, the sugar motif of a modified oligonucleotide is a gapmer and the intemucleoside linkages within the gap are all modified. In certain such embodiments, some or all of the intemucleoside linkages in the wings are unmodified phosphodiester intemucleoside linkages. In certain embodiments, the terminal intemucleoside linkages are modified. In certain embodiments, the sugar motif of a modified oligonucleotide is a gapmer, and the intemucleoside linkage motif comprises at least one phosphodiester intemucleoside linkage in at least one wing, wherein the at least one phosphodiester internucleoside linkage is not a terminal intemucleoside linkage, and the remaining intemucleoside linkages are phosphorothioate intemucleoside linkages. In certain such embodiments, all of the phosphorothioate internucleoside linkages are stereorandom. In certain embodiments, all of the phosphorothioate intemucleoside linkages in the wings are (Sp) phosphorothioates, and the gap comprises at least one Sp, Sp, Rp motif. In certain embodiments, populations of modified oligonucleotides are enriched for modified oligonucleotides comprising such intemucleoside linkage motifs.

In certain embodiments, modified oligonucleotides comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, or at least 19 phosphodiester intemucleoside linkages. In certain embodiments, modified oligonucleotides comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, or at least 19 phosphorothioate internucleoside linkages. In certain embodiments, modified oligonucleotides comprise at least 1, at least 2, at least 3, at least 4, or at least 5 phosphodiester internucleoside linkages and the remainder of the internucleoside linkages are phosphorothioate internucleoside linkages.

C. Certain Lengths

It is possible to increase or decrease the length of an oligonucleotide without eliminating activity. For example, in Woolf et al., Proc. Natl. Acad. Sci. USA, 1992, 89, 7305-7309, 1992), a series of oligonucleotides 13-25 nucleobases in length were tested for their ability to induce cleavage of a target nucleic acid in an oocyte injection model. Oligonucleotides 25 nucleobases in length with 8 or 11 mismatch bases near the ends of the oligonucleotides were able to direct specific cleavage of the target nucleic acid, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

In certain embodiments, oligonucleotides consist of 16 linked nucleosides. In certain embodiments, oligonucleotides consist of 17 linked nucleosides. In certain embodiments, oligonucleotides consist of 18 linked nucleosides. In certain embodiments, oligonucleotides consist of 19 linked nucleosides. In certain embodiments, oligonucleotides consist of 20 linked nucleosides.

D. Certain Modified Oligonucleotides

In certain embodiments, the above modifications (sugar, nucleobase, internucleoside linkage) are incorporated into a modified oligonucleotide. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such sugar gapmer oligonucleotides may comprise one or more modified nucleobase independent of the gapmer pattern of the sugar modifications. Unless otherwise indicated, all modifications are independent of nucleobase sequence.

E. Certain Populations of Modified Oligonucleotides

Populations of modified oligonucleotides in which all of the modified oligonucleotides of the population have the same molecular formula can be stereorandom populations or chirally enriched populations. All of the chiral centers of all of the modified oligonucleotides are stereorandom in a stereorandom population. In a chirally enriched population, at least one particular chiral center is not stereorandom in the modified oligonucleotides of the population. In certain embodiments, the modified oligonucleotides of a chirally enriched population are enriched for β-D ribosyl sugar moieties, and all of the phosphorothioate internucleoside linkages are stereorandom. In certain embodiments, the modified oligonucleotides of a chirally enriched population are enriched for both β-D ribosyl sugar moieties and at least one, particular phosphorothioate internucleoside linkage in a particular stereochemical configuration.

F. Nucleobase Sequence

In certain embodiments, oligonucleotides (unmodified or modified oligonucleotides) are further described by their nucleobase sequence. In certain embodiments oligonucleotides have a nucleobase sequence that is complementary to a second oligonucleotide or an identified reference nucleic acid, such as a target nucleic acid. In certain such embodiments, a portion of an oligonucleotide has a nucleobase sequence that is complementary to a second oligonucleotide or an identified reference nucleic acid, such as a target nucleic acid. In certain embodiments, the nucleobase sequence of a portion or entire length of an oligonucleotide is at least 50%, at least 60%, at least 70%, at least 80%, at least 85%, at least 90%, at least 95%, or 100% complementary to the second oligonucleotide or nucleic acid, such as a target nucleic acid.

### II. Certain Oligomeric Compounds

In certain embodiments, provided herein are oligomeric compounds, which consist of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, abasic nucleosides, modified or unmodified nucleosides, and two or more nucleosides that are independently modified or unmodified.

A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge, and clearance. In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy 2008, 16, 734-740), or a GalNAc cluster (e.g., WO2014/179620).

1. Conjugate Moieties

Conjugate moieties include, without limitation, intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, lipophilic groups, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial, or an antibiotic.

2. Conjugate Linkers

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain oligomeric compounds, a conjugate moiety is attached to an oligonucleotide via a more complex conjugate linker comprising one or more conjugate linker moieties, which are sub-units making up a conjugate linker. In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methyl cytosine, 4-N-benzoyl-5-methyl cytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxyribonucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate internucleoside linkage. In certain such embodiments, the cleavable moiety is 2′-deoxyadenosine.

B. Certain Terminal Groups

In certain embodiments, oligomeric compounds comprise one or more terminal groups. In certain such embodiments, oligomeric compounds comprise a stabilized 5′-phosphate. Stabilized 5′-phosphates include, but are not limited to 5′-phosphanates, including, but not limited to 5′-vinylphosphonates. In certain embodiments, terminal groups comprise one or more abasic nucleosides and/or inverted nucleosides. In certain embodiments, terminal groups comprise one or more 2′-linked nucleosides. In certain such embodiments, the 2′-linked nucleoside is an abasic nucleoside.

C. Oligomeric Duplexes

In certain embodiments, oligomeric compounds described herein comprise an oligonucleotide, having a nucleobase sequence complementary to that of a target nucleic acid. In certain embodiments, an oligomeric compound is paired with a second oligomeric compound to form an oligomeric duplex. Such oligomeric duplexes comprise a first oligomeric compound having a portion complementary to a target nucleic acid and a second oligomeric compound having a portion complementary to the first oligomeric compound. In certain embodiments, the first oligomeric compound of an oligomeric duplex comprises or consists of (1) a modified or unmodified oligonucleotide and optionally a conjugate group and (2) a second modified or unmodified oligonucleotide and optionally a conjugate group. Either or both oligomeric compounds of an oligomeric duplex may comprise a conjugate group. The oligonucleotides of each oligomeric compound of an oligomeric duplex may include non-complementary overhanging nucleosides.

D. Antisense Activity

In certain embodiments, oligomeric compounds and oligomeric duplexes are capable of hybridizing to a target nucleic acid, resulting in at least one antisense activity; such oligomeric compounds and oligomeric duplexes are antisense compounds. In certain embodiments, antisense compounds have antisense activity when they reduce, modulate, or increase the amount or activity of a target nucleic acid by 25% or more in the standard cell assay. In certain embodiments, antisense compounds selectively affect one or more target nucleic acid. Such antisense compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In certain antisense activities, hybridization of an antisense compound to a target nucleic acid results in recruitment of a protein that cleaves the target nucleic acid. For example, certain antisense compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, provided herein are antisense compounds that are sufficiently “DNA-like” to elicit RNase H activity. In certain embodiments, one or more non-DNA-like nucleoside in the gap of a gapmer is tolerated.

In certain antisense activities, an antisense compound or a portion of an antisense compound is loaded into an RNA-induced silencing complex (RISC), ultimately resulting in cleavage of the target nucleic acid. For example, certain antisense compounds result in cleavage of the target nucleic acid by Argonaute. Antisense compounds that are loaded into RISC are RNAi compounds. RNAi compounds may be double-stranded (siRNA) or single-stranded (ssRNA).

In certain embodiments, hybridization of an antisense compound to a target nucleic acid does not result in recruitment of a protein that cleaves that target nucleic acid. In certain embodiments, hybridization of the antisense compound to the target nucleic acid results in alteration of splicing of the target nucleic acid. In certain embodiments, hybridization of an antisense compound to a target nucleic acid results in inhibition of a binding interaction between the target nucleic acid and a protein or other nucleic acid. In certain embodiments, hybridization of an antisense compound to a target nucleic acid results in alteration of translation of the target nucleic acid. In certain embodiments, hybridization of an antisense compound to a target nucleic acid results in a reduced amount or level of RNA that includes an NIE. In certain embodiments, hybridization of an antisense compound to a target nucleic acid results in exon inclusion. In certain embodiments, hybridization of an antisense compound to a target nucleic acid results in an increase in the amount or activity of a target nucleic acid. In certain embodiments, hybridization of an antisense compound complementary to a target nucleic acid results in alteration of splicing, leading to the inclusion of an exon in the mRNA.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein and/or a phenotypic change in a cell or subject.

### III. Certain Target Nucleic Acids

In certain embodiments, oligomeric compounds comprise or consist of an oligonucleotide comprising a portion that is complementary to a target nucleic acid. In certain embodiments, the target nucleic acid is an endogenous RNA molecule. In certain embodiments, the target nucleic acid encodes a protein. In certain such embodiments, the target nucleic acid is selected from: a mature mRNA and a pre-mRNA, including intronic, exonic and untranslated regions. In certain embodiments, the target nucleic acid is a mature mRNA. In certain embodiments, the target nucleic acid is a pre-mRNA. In certain embodiments, the target region is entirely within an intron. In certain embodiments, the target region spans an intron/exon junction. In certain embodiments, the target region is at least 50% within an intron.

A. Complementarity/Mismatches to the Target Nucleic Acid

It is possible to introduce mismatch bases without eliminating activity. For example, Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo. Maher and Dolnick (Nuc. Acid. Res. 16:3341-3358, 1988) tested a series of tandem 14 nucleobase oligonucleotides, and a 28 and 42 nucleobase oligonucleotides comprised of the sequence of two or three of the tandem oligonucleotides, respectively, for their ability to arrest translation of human DHFR in a rabbit reticulocyte assay. Each of the three 14 nucleobase oligonucleotides alone was able to inhibit translation, albeit at a more modest level than the 28 or 42 nucleobase oligonucleotides.

In certain embodiments, oligonucleotides are complementary to the target nucleic acid over the entire length of the oligonucleotide. In certain embodiments, oligonucleotides are 99%, 95%, 90%, 85%, or 80% complementary to the target nucleic acid. In certain embodiments, oligonucleotides are at least 80% complementary to the target nucleic acid over the entire length of the oligonucleotide and comprise a portion that is 100% or fully complementary to a target nucleic acid. In certain embodiments, the portion of full complementarity is 6, 7, 8, 9, 10, 11, 12, 13, 14, 15, 16, 17, 18, 19, or 20 nucleobases in length.

In certain embodiments, oligonucleotides comprise one or more mismatched nucleobases relative to the target nucleic acid. In certain embodiments, the mismatch is at position 1, 2, 3, 4, 5, 6, 7, 8, 9, 10, 11, 12, 13, 14, 15, 16, 17, 18, 19, or 20 from the 5′-end of the oligonucleotide.

B. SCN1A

In certain embodiments, oligomeric compounds comprise or consist of a modified oligonucleotide that is complementary to a target nucleic acid encoding SCN1A, or a portion thereof. In certain embodiments, the SCN1A target nucleic acid has the nucleobase sequence set forth in SEQ ID NO: 1 (the complement of GENBANK Accession No. NC_000002.12 truncated from nucleotides 165982001 to 166152000). In certain embodiments, the SCN1A target nucleic acid has the nucleobase sequence set forth in SEQ ID NO: 2 (GENBANK Accession No. NM_001165963.2).

In certain embodiments, contacting a cell or subject with an oligomeric compound complementary to SEQ ID NO: 1 or SEQ ID NO: 2 modulates splicing of SCN1A RNA in a cell or a subject. In certain embodiments, contacting a cell or a subject with an oligomeric compound complementary to SEQ ID NO: 1 or SEQ ID NO: 2 increases the amount of SCN1A RNA and/or protein. In certain embodiments, contacting a cell or a subject with an oligomeric compound complementary to SEQ ID NO: 1 or SEQ ID NO: 2 reduces the amount of SCN1A RNA including a NIE. In certain embodiments, contacting a cell or a subject with an oligomeric compound complementary to SEQ ID NO: 1 or SEQ ID NO: 2 increases the amount of SCN1A RNA excluding a NIE. In certain embodiments, the NIE is NIE-1. In certain embodiments, the oligomeric compound comprises or consists of a modified oligonucleotide.

In certain embodiments, contacting a cell in a subject with an oligomeric compound complementary to SEQ ID NO: 1 or SEQ ID NO: 2 ameliorates one or more symptom of encephalopathy. In certain embodiments, the encephalopathy is Dravet Syndrome. In certain embodiments, the symptom is any of prolonged or frequent seizures, sudden unexpected death in epilepsy, status epilepticus, behavioral and developmental delays, movement and balance dysfunctions, orthopedic conditions, motor and cognitive dysfunctions, delayed language and speech issues, visual motor integration dysfunctions, visual perception dysfunctions, executive dysfunctions, growth and nutrition issues, sleeping difficulties, chronic infections, sensory integration disorders, and dysautonomia.

C. Certain Target Nucleic Acids in Certain Tissues

In certain embodiments, oligomeric compounds comprise or consist of an oligonucleotide comprising a portion that is complementary to a target nucleic acid, wherein the target nucleic acid is expressed in a pharmacologically relevant tissue. In certain embodiments, the pharmacologically relevant tissues are the cells and tissues that comprise the central nervous system (CNS). Such tissues include brain tissues, such as, cerebral cortex.

### IV. Certain Pharmaceutical Compositions

In certain embodiments, described herein are pharmaceutical compositions comprising one or more oligomeric compounds. In certain embodiments, the one or more oligomeric compounds each consists of a modified oligonucleotide. In certain embodiments, the pharmaceutical composition comprises a pharmaceutically acceptable diluent or carrier. In certain embodiments, a pharmaceutical composition comprises or consists of a sterile saline solution and one or more oligomeric compound. In certain embodiments, the sterile saline is pharmaceutical grade saline. In certain embodiments, a pharmaceutical composition comprises or consists of one or more oligomeric compound and sterile water. In certain embodiments, the sterile water is pharmaceutical grade water. In certain embodiments, a pharmaceutical composition comprises or consists of one or more oligomeric compound and phosphate-buffered saline (PBS). In certain embodiments, the sterile PBS is pharmaceutical grade PBS. In certain embodiments, a pharmaceutical composition comprises or consists of one or more oligomeric compound and artificial cerebrospinal fluid (“artificial CSF” or “aCSF”). In certain embodiments, the artificial cerebrospinal fluid is pharmaceutical grade.

In certain embodiments, a pharmaceutical composition comprises a modified oligonucleotide and artificial cerebrospinal fluid. In certain embodiments, a pharmaceutical composition consists of a modified oligonucleotide and artificial cerebrospinal fluid. In certain embodiments, a pharmaceutical composition consists essentially of a modified oligonucleotide and artificial cerebrospinal fluid. In certain embodiments, the artificial cerebrospinal fluid is pharmaceutical grade.

In certain embodiments, pharmaceutical compositions comprise one or more oligomeric compound and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, oligomeric compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an oligomeric compound encompass any pharmaceutically acceptable salts of the oligomeric compound, esters of the oligomeric compound, or salts of such esters. In certain embodiments, pharmaceutical compositions comprising oligomeric compounds comprising one or more oligonucleotide, upon administration to a subject, including a human, are capable of providing (directly or indirectly) the biologically active metabolite or residue thereof. Accordingly, for example, the disclosure is also drawn to pharmaceutically acceptable salts of oligomeric compounds, prodrugs, pharmaceutically acceptable salts of such prodrugs, and other bioequivalents. Suitable pharmaceutically acceptable salts include, but are not limited to, sodium and potassium salts. In certain embodiments, prodrugs comprise one or more conjugate group attached to an oligonucleotide, wherein the conjugate group is cleaved by endogenous nucleases within the body. In certain embodiments, prodrugs comprise one or more conjugate group attached to an oligonucleotide, wherein the conjugate group is cleaved by endogenous nucleases within the body.

Lipid moieties have been used in nucleic acid therapies in a variety of methods. In certain such methods, the nucleic acid, such as an oligomeric compound, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents comprising an oligomeric compound provided herein to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal (IT), intracerebroventricular (ICV), etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

Under certain conditions, certain compounds disclosed herein act as acids. Although such compounds may be drawn or described in protonated (free acid) form, or ionized and in association with a cation (salt) form, aqueous solutions of such compounds exist in equilibrium among such forms. For example, a phosphate linkage of an oligonucleotide in aqueous solution exists in equilibrium among free acid, anion and salt forms. Unless otherwise indicated, compounds described herein are intended to include all such forms. Moreover, certain oligonucleotides have several such linkages, each of which is in equilibrium. Thus, oligonucleotides in solution exist in an ensemble of forms at multiple positions all at equilibrium. The term “oligonucleotide” is intended to include all such forms. Drawn structures necessarily depict a single form. Nevertheless, unless otherwise indicated, such drawings are likewise intended to include corresponding forms. Herein, a structure depicting the free acid of a compound followed by the term “or a salt thereof” expressly includes all such forms that may be fully or partially protonated/de-protonated/in association with a cation. In certain instances, one or more specific cation is identified.

In certain embodiments, modified oligonucleotides or oligomeric compounds are in aqueous solution with sodium. In certain embodiments, modified oligonucleotides or oligomeric compounds are in aqueous solution with potassium. In certain embodiments, modified oligonucleotides or oligomeric compounds are in PBS. In certain embodiments, modified oligonucleotides or oligomeric compounds are in water. In certain such embodiments, the pH of the solution is adjusted with NaOH and/or HCl to achieve a desired pH.

Herein, certain specific doses are described. A dose may be in the form of a dosage unit. For clarity, a dose (or dosage unit) of a modified oligonucleotide or an oligomeric compound in milligrams indicates the mass of the free acid form of the modified oligonucleotide or oligomeric compound. As described above, in aqueous solution, the free acid is in equilibrium with anionic and salt forms. However, for the purpose of calculating dose, it is assumed that the modified oligonucleotide or oligomeric compound exists as a solvent-free, sodium-acetate free, anhydrous, free acid. For example, where a modified oligonucleotide or an oligomeric compound is in solution comprising sodium (e.g., saline), the modified oligonucleotide or oligomeric compound may be partially or fully de-protonated and in association with Na+ ions. However, the mass of the protons are nevertheless counted toward the weight of the dose, and the mass of the Na+ ions are not counted toward the weight of the dose. Thus, for example, a dose, or dosage unit, of 10 mg of Compound No. 1429226, equals the number of fully protonated molecules that weighs 10 mg. This would be equivalent to 10.5 mg of solvent-free, sodium acetate-free, anhydrous sodiated Compound No. 1429226. When an oligomeric compound comprises a conjugate group, the mass of the conjugate group is included in calculating the dose of such oligomeric compound. If the conjugate group also has an acid, the conjugate group is likewise assumed to be fully protonated for the purpose of calculating dose.

### V. Certain Compositions

1. Compound No: 1429226

In certain embodiments, Compound No. 1429226 is characterized as a modified oligonucleotide having a sequence of (from 5′ to 3′) AGTTGGAGCAAGATTATC (SEQ ID NO: 41), wherein each nucleoside comprises a 2′-NMA sugar moiety, each intemucleoside linkage is a phosphorothioate internucleoside linkage, and each cytosine is a 5-methyl cytosine.

In certain embodiments, Compound No. 1429226 is represented by the following chemical notation (5′ to 3′): Aₙₛ Gₙₛ Tₙₛ Tₙₛ Gₙₛ Gₙₛ Aₙₛ Gₙₛ^{m}Cₙₛ Aₙₛ Aₙₛ Gₙₛ Aₙₛ Tₙₛ Tₙₛ Aₙₛ Tₙₛ^{m}Cₙ (SEQ ID NO: 41);

wherein,

A=an adenine nucleobase,

^{m}C=a 5-methyl cytosine nucleobase,

G=a guanine nucleobase,

T=a thymine nucleobase,

n=a 2′-NMA sugar moiety, and

s=a phosphorothioate internucleoside linkage.

In certain embodiments, Compound No. 1429226 is represented by the following chemical structure:

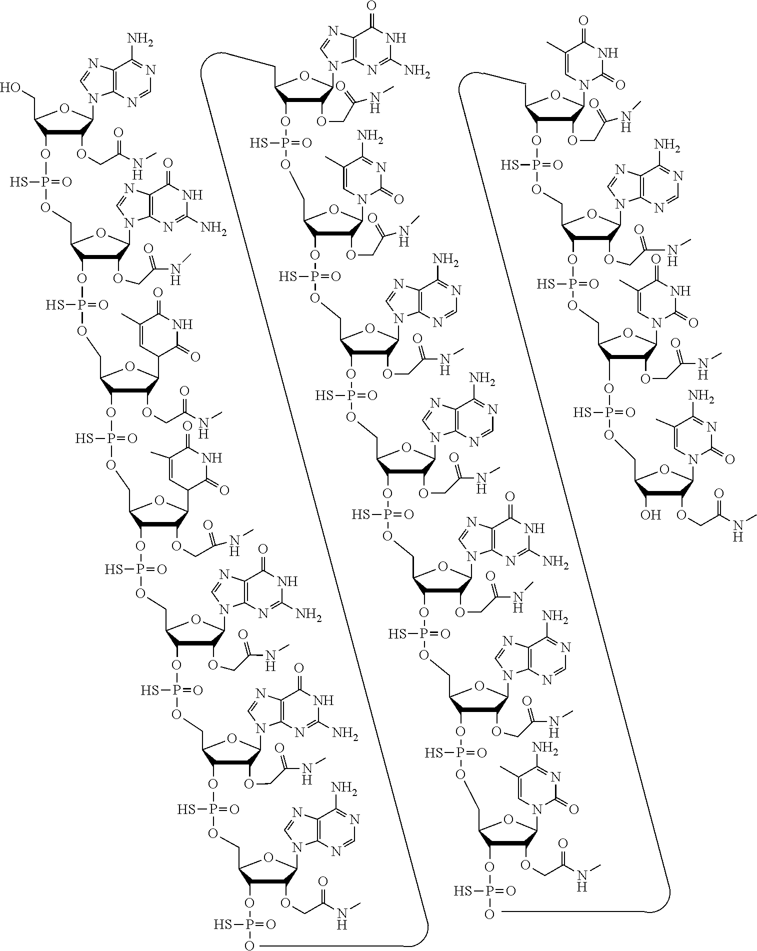

### Structure 1. Compound No. 1429226

In certain embodiments, the sodium salt of Compound No. 1429226 is represented by the following chemical

structure:

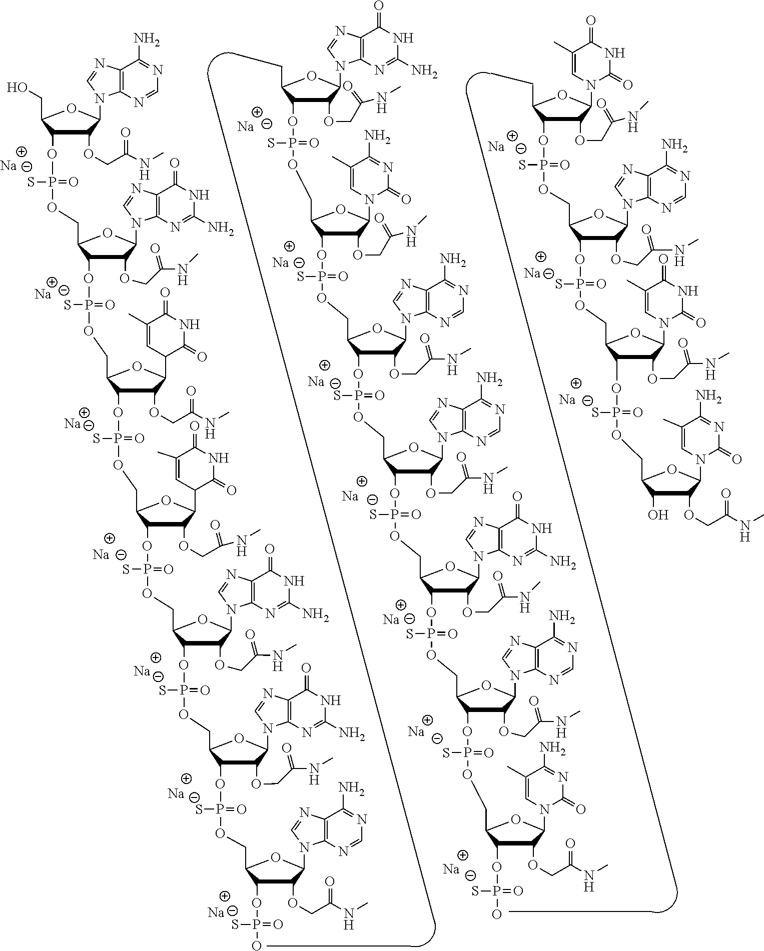

### Structure 2. The Sodium Salt of Compound No. 1429226

### Nonlimiting Disclosure and Incorporation by Reference

Each of the literature and patent publications listed herein is incorporated by reference in its entirety. While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar moiety (2′-OH in place of one 2′-H of DNA) or as an RNA having a modified base (thymine (methylated uracil) in place of a uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG,” wherein ^{m}C indicates a cytosine base comprising a methyl group at the 5-position.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as a or 3 such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms, unless specified otherwise. Likewise, all cis- and trans-isomers and tautomeric forms of the compounds herein are also included unless otherwise indicated. Oligomeric compounds described herein include chirally pure or enriched mixtures as well as racemic mixtures. For example, oligomeric compounds having a plurality of phosphorothioate internucleoside linkages include such compounds in which chirality of the phosphorothioate intemucleoside linkages is controlled or is random. Unless otherwise indicated, compounds described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXAMPLES

The following examples illustrate certain embodiments of the present disclosure and are not limiting. Moreover, where specific embodiments are provided, the inventors have contemplated generic application of those specific embodiments.

### Example 1: Activity of Modified Oligonucleotides Targeting Human SCN1A in HepG2 Cells, Single Dose, In Vitro

Modified oligonucleotides complementary to a human SCN1A nucleic acid were synthesized and tested for their effect on SCN1A RNA levels in vitro. The modified oligonucleotides were tested in a series of experiments using the same culture conditions.

The modified oligonucleotides in the tables below are 18 nucleosides in length. Each nucleoside comprises a 2′-MOE sugar moiety. The intemucleoside linkages throughout each modified oligonucleotide are phosphorothioate internucleoside linkages. All cytosine nucleobases throughout each modified oligonucleotide are 5-methylcytosines.

Each modified oligonucleotide listed in the tables below is 100% complementary to either the human SCN1A genomic sequence, designated herein as SEQ ID NO: 1 (the complement of GENBANK Accession No. NC_000002.12 truncated from nucleotides 165982001 to 166152000) or to the human SCN1A mRNA, designated herein as SEQ ID NO: 2 (GENBANK Accession No. NM_001165963.2) or to both. ‘N/A’ indicates that the modified oligonucleotide is not complementary to that particular target sequence with 100% complementarity. “Start site” indicates the 5′-most nucleoside of the target sequence to which the modified oligonucleotide is complementary. “Stop site” indicates the 3′-most nucleoside of the target sequence to which the modified oligonucleotide is complementary.

Cultured HepG2 cells at a density of 20,000 cells per well were transfected using electroporation with 4000 nM of modified oligonucleotide. After a treatment period of approximately 24 hours, RNA was isolated from the cells and SCN1A RNA levels were measured by quantitative real-time RTPCR. Human primer probe set RTS40976 (forward sequence CCAAGAAGGCTGGAATATCTTTG, designated herein as SEQ ID NO: 15; reverse sequence GCCAACTTGAAAACTCGCA, designated herein as SEQ ID NO: 16; probe sequence ACCAGGCTAAGCGTCACAATAAAACCG, designated herein as SEQ ID NO: 17) was used to measure SCN1A RNA levels. SCN1A RNA levels were normalized to total RNA content, as measured by RIBOGREEN@. SCN1A RNA is presented as % of the average of untreated control (% UTC). As shown in the tables below, certain modified oligonucleotides complementary to SCN1A RNA increased the amount of human SCN1A RNA compared to untreated control. Each of Tables 1-6 represents a different experiment.

TABLE 1
Effect of modified oligonucleotides on amount of human SCNIA RNA
in HepG2 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | SCNIA | SEQ |
| Compound | Start | Stop | Start | Stop | | (% | ID |
| Number | Site | Site | Site | Site | Sequence (5′ to 3′) | UTC) | No. |
| 1262739 | 2869 | 2886 | N/A | N/A | AACAGTTCCATGAGTTTC | 57 | 42 |
| 1262745 | 2875 | 2892 | N/A | N/A | TGGAGGAACAGTTCCATG | 90 | 43 |
| 1262751 | 2881 | 2898 | N/A | N/A | TTAATCTGGAGGAACAGT | 130 | 44 |
| 1262757 | 2887 | 2904 | N/A | N/A | GAAGTGTTAATCTGGAGG | 129 | 45 |
| 1262763 | 2893 | 2910 | N/A | N/A | ACCCCTGAAGTGTTAATC | 93 | 46 |
| 1262769 | 2899 | 2916 | N/A | N/A | TCCATAACCCCTGAAGTG | 89 | 47 |
| 1262775 | 2905 | 2922 | N/A | N/A | CCAGCTTCCATAACCCCT | 87 | 48 |
| 1262781 | 2912 | 2929 | N/A | N/A | GCTTCCTCCAGCTTCCAT | 54 | 49 |
| 1262787 | 2925 | 2942 | N/A | N/A | TAGTAAAAGCTCAGCTTC | 75 | 50 |
| 1262793 | 2931 | 2948 | N/A | N/A | AAGATGTAGTAAAAGCTC | 108 | 51 |
| 1262799 | 78339 | 78356 | 457 | 474 | TACCATTTATTCTGCATA | 79 | 52 |
| 1262805 | 78359 | 78376 | 477 | 494 | GTCATCCTGCACATTTTA | 84 | 53 |
| 1262811 | 160818 | 160835 | 6589 | 6606 | AGGAGTCCTGTTGATAAA | 102 | 54 |
| 1262817 | 160825 | 160842 | 6596 | 6613 | CTCCTAAAGGAGTCCTGT | 66 | 55 |
| 1262823 | 160839 | 160856 | 6610 | 6627 | AGTTTGGCATTGACCTCC | 54 | 56 |
| 1262829 | 160874 | 160891 | 6645 | 6662 | GCACTGACCTTAAGGAGA | 126 | 57 |
| 1262835 | 160884 | 160901 | 6655 | 6672 | CTTATTGTAGGCACTGAC | 69 | 58 |
| 1262841 | 160927 | 160944 | 6698 | 6715 | CCCCTTTACACAGAGTCA | 61 | 59 |
| 1262847 | 160954 | 160971 | 6725 | 6742 | AACAGTAACCTCCTGTCA | 85 | 60 |
| 1262853 | 160966 | 160983 | 6737 | 6754 | GCTGGTAGTGAGAACAGT | 93 | 61 |
| 1262859 | 160974 | 160991 | 6745 | 6762 | CAGTGTCAGCTGGTAGTG | 84 | 62 |
| 1262865 | 161002 | 161019 | 6773 | 6790 | GACTAGCCATTGTGCATC | 113 | 63 |
| 1262871 | 161008 | 161025 | 6779 | 6796 | CAGTCTGACTAGCCATTG | 74 | 64 |
| 1262877 | 161014 | 161031 | 6785 | 6802 | TCCCTACAGTCTGACTAG | 72 | 65 |
| 1262883 | 161020 | 161037 | 6791 | 6808 | AACTGGTCCCTACAGTCT | 73 | 66 |
| 1262889 | 161031 | 161048 | 6802 | 6819 | GCACCCCTTGAAACTGGT | 114 | 67 |
| 1262895 | 161037 | 161054 | 6808 | 6825 | AGGTTTGCACCCCTTGAA | 85 | 68 |
| 1262901 | 161087 | 161104 | 6858 | 6875 | GATACAATTACTACACTA | 81 | 69 |
| 1262907 | 161150 | 161167 | 6921 | 6938 | GATGAATCCACTAACAGA | 32 | 70 |
| 1262913 | 161231 | 161248 | 7002 | 7019 | TAGAGGTCCTTAGCCTAT | 75 | 71 |
| 1262919 | 161241 | 161258 | 7012 | 7029 | ATACCTGTTATAGAGGTC | 74 | 72 |
| 1262925 | 161247 | 161264 | 7018 | 7035 | GGTGGCATACCTGTTATA | 62 | 73 |
| 1262931 | 161258 | 161275 | 7029 | 7046 | CATACCCCCCAGGTGGCA | 70 | 74 |
| 1262937 | 161264 | 161281 | 7035 | 7052 | GGTTGCCATACCCCCCAG | 40 | 75 |
| 1262943 | 161270 | 161287 | 7041 | 7058 | CCATGTGGTTGCCATACC | 72 | 76 |
| 1262949 | 161293 | 161310 | 7064 | 7081 | ACGACTTTGTGTAGCTGG | 90 | 77 |
| 1262955 | 161299 | 161316 | 7070 | 7087 | CAAACCACGACTTTGTGT | 57 | 78 |
| 1262961 | 161305 | 161322 | 7076 | 7093 | CTCATGCAAACCACGACT | 61 | 79 |
| 1262967 | 161313 | 161330 | 7084 | 7101 | AGCATGCCCTCATGCAAA | 53 | 80 |
| 1262973 | 161319 | 161336 | 7090 | 7107 | AAGTGCAGCATGCCCTCA | 135 | 81 |
| 1262979 | 161326 | 161343 | 7097 | 7114 | GATCTCTAAGTGCAGCAT | 76 | 82 |
| 1262985 | 161400 | 161417 | 7171 | 7188 | ATCACCCAATTACCCCTC | 56 | 83 |
| 1262991 | 161406 | 161423 | 7177 | 7194 | CCACTTATCACCCAATTA | 27 | 84 |
| 1262997 | 161412 | 161429 | 7183 | 7200 | GCACCTCCACTTATCACC | 83 | 85 |
| 1263003 | 161439 | 161456 | 7210 | 7227 | TGGATTTCGCAAAACAAG | 57 | 86 |
| 1263009 | 161461 | 161478 | 7232 | 7249 | ATAATCTACTTGGTCTAG | 81 | 87 |
| 1263015 | 161477 | 161494 | 7248 | 7265 | ACTGGCCTACCCACAAAT | 67 | 88 |
| 1263021 | 161483 | 161500 | 7254 | 7271 | AGATTTACTGGCCTACCC | 104 | 89 |
| 1263027 | 161495 | 161512 | 7266 | 7283 | TTGCACCTGCTAAGATTT | 101 | 90 |
| 1263033 | 161501 | 161518 | 7272 | 7289 | TGAAGTTTGCACCTGCTA | 115 | 91 |
| 1263039 | 161601 | 161618 | 7372 | 7389 | GTCTTCTGGCGGTGGAGG | 83 | 92 |
| 1263045 | 161607 | 161624 | 7378 | 7395 | AATTCAGTCTTCTGGCGG | 65 | 93 |
| 1263051 | 161667 | 161684 | 7438 | 7455 | CCGAAGATGGCTAAACAA | 51 | 94 |
| 1263057 | 161673 | 161690 | 7444 | 7461 | TGAGAGCCGAAGATGGCT | 64 | 95 |
| 1263063 | 161680 | 161697 | 7451 | 7468 | ACCTTGCTGAGAGCCGAA | 66 | 96 |
| 1263069 | 161690 | 161707 | 7461 | 7478 | TACAGTGTCAACCTTGCT | 113 | 97 |
| 1263075 | 161743 | 161760 | 7514 | 7531 | GCACCACAGGGTAAAATG | 58 | 98 |
| 1263081 | 161779 | 161796 | 7550 | 7567 | TACTGTGCTTAGGTCATT | 83 | 99 |
| 1263087 | 161828 | 161845 | 7599 | 7616 | GTAAAGCTTGCACTCTAC | 87 | 100 |
| 1263093 | 161836 | 161853 | 7607 | 7624 | TTACCTGTGTAAAGCTTG | 40 | 101 |
| 1263099 | 161879 | 161896 | 7650 | 7667 | GATAGCATCCAAACTATC | 89 | 102 |
| 1263105 | 161887 | 161904 | 7658 | 7675 | CATGCATTGATAGCATCC | 87 | 103 |
| 1263111 | 161965 | 161982 | 7736 | 7753 | TACCACTGACATATGGTT | 71 | 104 |
| 1263117 | 162030 | 162047 | 7801 | 7818 | AAGTGCTGCAAACTATTG | 97 | 105 |
| 1263123 | 162092 | 162109 | 7863 | 7880 | ACAGTCTGGCTATATACC | 109 | 106 |
| 1263129 | 162098 | 162115 | 7869 | 7886 | GTCTGTACAGTCTGGCTA | 75 | 107 |
| 1263135 | 162129 | 162146 | 7900 | 7917 | AATAGGTTAAGCAGTGTG | 66 | 108 |
| 1263141 | 162249 | 162266 | 8020 | 8037 | ATTGTGATATCAACCTGA | 65 | 109 |
| 1263147 | 162337 | 162354 | 8108 | 8125 | AATCTACAACTACCCAGT | 82 | 110 |
| 1263153 | 162482 | 162499 | 8253 | 8270 | TAGTGCAGGTACTACCAG | 75 | ill |
| 1263159 | 162488 | 162505 | 8259 | 8276 | TTCAGTTAGTGCAGGTAC | 84 | 112 |
| 1263165 | 162501 | 162518 | 8272 | 8289 | GCACTACCTTCAATTCAG | 88 | 113 |
| 1263171 | 162578 | 162595 | 8349 | 8366 | AACAATCTAGAGCAGCAT | 105 | 114 |
| 1263177 | 162638 | 162655 | 8409 | 8426 | TATAAGTTGACTACTCTG | 71 | 115 |
| 1263183 | 162656 | 162673 | 8427 | 8444 | TCCTGATGTAATTGACTA | 54 | 116 |
| 1263189 | 162696 | 162713 | 8467 | 8484 | AGAGGAGCCTATGGTTTG | 76 | 117 |
| 1263195 | 162735 | 162752 | 8506 | 8523 | AGTTCACGAATACAGTTT | 51 | 118 |
| 1263201 | 162741 | 162758 | 8512 | 8529 | GCATGCAGTTCACGAATA | 87 | 119 |

TABLE 2
Effect of modified oligonucleotides on amount of human SCNIA RNA
in HepG2 cells

| | SEQ | SEQ | | | | | |
|---|---|---|---|---|---|---|---|
| | ID | ID | SEQ | SEQ | | | |
| | NO: 1 | NO: 1 | ID NO: | ID NO: | | SCNIA | SEQ |
| Compound | Start | Stop | 2 Start | 2 Stop | | (% | ID |
| Number | Site | Site | Site | Site | Sequence (5′ to 3′) | UTC) | No. |
| 1262740 | 2870 | 2887 | N/A | N/A | GAACAGTTCCATGAGTTT | 62 | 120 |
| 1262746 | 2876 | 2893 | N/A | N/A | CTGGAGGAACAGTTCCAT | 82 | 121 |
| 1262752 | 2882 | 2899 | N/A | N/A | GTTAATCTGGAGGAACAG | 92 | 122 |
| 1262758 | 2888 | 2905 | N/A | N/A | TGAAGTGTTAATCTGGAG | 71 | 123 |
| 1262764 | 2894 | 2911 | N/A | N/A | AACCCCTGAAGTGTTAAT | 83 | 124 |
| 1262770 | 2900 | 2917 | N/A | N/A | TTCCATAACCCCTGAAGT | 112 | 125 |
| 1262776 | 2906 | 2923 | N/A | N/A | TCCAGCTTCCATAACCCC | 82 | 126 |
| 1262782 | 2919 | 2936 | N/A | N/A | AAGCTCAGCTTCCTCCAG | 70 | 127 |
| 1262788 | 2926 | 2943 | N/A | N/A | GTAGTAAAAGCTCAGCTT | 82 | 128 |
| 1262794 | 2932 | 2949 | N/A | N/A | AAAGATGTAGTAAAAGCT | 75 | 129 |
| 1262800 | 78342 | 78359 | 460 | 477 | AATTACCATTTATTCTGC | 37 | 130 |
| 1262806 | 78360 | 78377 | 478 | 495 | TGTCATCCTGCACATTTT | 124 | 131 |
| 1262812 | 160819 | 160836 | 6590 | 6607 | AAGGAGTCCTGTTGATAA | 58 | 132 |
| 1262818 | 160828 | 160845 | 6599 | 6616 | GACCTCCTAAAGGAGTCC | 66 | 133 |
| 1262824 | 160840 | 160857 | 6611 | 6628 | CAGTTTGGCATTGACCTC | 54 | 134 |
| 1262830 | 160879 | 160896 | 6650 | 6667 | TGTAGGCACTGACCTTAA | 72 | 135 |
| 1262836 | 160885 | 160902 | 6656 | 6673 | TCTTATTGTAGGCACTGA | 63 | 136 |
| 1262842 | 160949 | 160966 | 6720 | 6737 | TAACCTCCTGTCAAGGTC | 88 | 137 |
| 1262848 | 160955 | 160972 | 6726 | 6743 | GAACAGTAACCTCCTGTC | 55 | 138 |
| 1262854 | 160969 | 160986 | 6740 | 6757 | TCAGCTGGTAGTGAGAAC | 113 | 139 |
| 1262860 | 160991 | 161008 | 6762 | 6779 | GTGCATCTTATCTTCAGC | 109 | 140 |
| 1262866 | 161003 | 161020 | 6774 | 6791 | TGACTAGCCATTGTGCAT | 54 | 141 |
| 1262872 | 161009 | 161026 | 6780 | 6797 | ACAGTCTGACTAGCCATT | 92 | 142 |
| 1262878 | 161015 | 161032 | 6786 | 6803 | GTCCCTACAGTCTGACTA | 129 | 143 |
| 1262884 | 161021 | 161038 | 6792 | 6809 | AAACTGGTCCCTACAGTC | 87 | 144 |
| 1262890 | 161032 | 161049 | 6803 | 6820 | TGCACCCCTTGAAACTGG | 92 | 145 |
| 1262896 | 161038 | 161055 | 6809 | 6826 | CAGGTTTGCACCCCTTGA | 65 | 146 |
| 1262902 | 161088 | 161105 | 6859 | 6876 | GGATACAATTACTACACT | 79 | 147 |
| 1262908 | 161151 | 161168 | 6922 | 6939 | AGATGAATCCACTAACAG | 44 | 148 |
| 1262914 | 161232 | 161249 | 7003 | 7020 | ATAGAGGTCCTTAGCCTA | 54 | 149 |
| 1262920 | 161242 | 161259 | 7013 | 7030 | CATACCTGTTATAGAGGT | 106 | 150 |
| 1262926 | 161248 | 161265 | 7019 | 7036 | AGGTGGCATACCTGTTAT | 78 | 151 |
| 1262932 | 161259 | 161276 | 7030 | 7047 | CCATACCCCCCAGGTGGC | 88 | 152 |
| 1262938 | 161265 | 161282 | 7036 | 7053 | TGGTTGCCATACCCCCCA | 41 | 153 |
| 1262944 | 161271 | 161288 | 7042 | 7059 | GCCATGTGGTTGCCATAC | 73 | 154 |
| 1262950 | 161294 | 161311 | 7065 | 7082 | CACGACTTTGTGTAGCTG | 63 | 155 |
| 1262956 | 161300 | 161317 | 7071 | 7088 | GCAAACCACGACTTTGTG | 48 | 156 |
| 1262962 | 161306 | 161323 | 7077 | 7094 | CCTCATGCAAACCACGAC | 68 | 157 |
| 1262968 | 161314 | 161331 | 7085 | 7102 | CAGCATGCCCTCATGCAA | 77 | 158 |
| 1262974 | 161320 | 161337 | 7091 | 7108 | TAAGTGCAGCATGCCCTC | 80 | 159 |
| 1262980 | 161328 | 161345 | 7099 | 7116 | ATGATCTCTAAGTGCAGC | 114 | 160 |
| 1262986 | 161401 | 161418 | 7172 | 7189 | TATCACCCAATTACCCCT | 59 | 161 |
| 1262992 | 161407 | 161424 | 7178 | 7195 | TCCACTTATCACCCAATT | 65 | 162 |
| 1262998 | 161413 | 161430 | 7184 | 7201 | AGCACCTCCACTTATCAC | 139 | 163 |
| 1263004 | 161441 | 161458 | 7212 | 7229 | GCTGGATTTCGCAAAACA | 84 | 164 |
| 1263010 | 161462 | 161479 | 7233 | 7250 | AATAATCTACTTGGTCTA | 83 | 165 |
| 1263016 | 161478 | 161495 | 7249 | 7266 | TACTGGCCTACCCACAAA | 47 | 166 |
| 1263022 | 161484 | 161501 | 7255 | 7272 | AAGATTTACTGGCCTACC | 63 | 167 |
| 1263028 | 161496 | 161513 | 7267 | 7284 | TTTGCACCTGCTAAGATT | 68 | 168 |
| 1263034 | 161502 | 161519 | 7273 | 7290 | ATGAAGTTTGCACCTGCT | 75 | 169 |
| 1263040 | 161602 | 161619 | 7373 | 7390 | AGTCTTCTGGCGGTGGAG | 61 | 170 |
| 1263046 | 161608 | 161625 | 7379 | 7396 | CAATTCAGTCTTCTGGCG | 73 | 171 |
| 1263052 | 161668 | 161685 | 7439 | 7456 | GCCGAAGATGGCTAAACA | 102 | 172 |
| 1263058 | 161674 | 161691 | 7445 | 7462 | CTGAGAGCCGAAGATGGC | 83 | 173 |
| 1263064 | 161681 | 161698 | 7452 | 7469 | AACCTTGCTGAGAGCCGA | 109 | 174 |
| 1263070 | 161691 | 161708 | 7462 | 7479 | ATACAGTGTCAACCTTGC | 93 | 175 |
| 1263076 | 161744 | 161761 | 7515 | 7532 | TGCACCACAGGGTAAAAT | 53 | 176 |
| 1263082 | 161780 | 161797 | 7551 | 7568 | ATACTGTGCTTAGGTCAT | 52 | 177 |
| 1263088 | 161829 | 161846 | 7600 | 7617 | TGTAAAGCTTGCACTCTA | 48 | 178 |
| 1263094 | 161867 | 161884 | 7638 | 7655 | ACTATCTATAAATGGTAC | 135 | 179 |
| 1263100 | 161880 | 161897 | 7651 | 7668 | TGATAGCATCCAAACTAT | 56 | 180 |
| 1263106 | 161888 | 161905 | 7659 | 7676 | ACATGCATTGATAGCATC | 29 | 181 |
| 1263112 | 161966 | 161983 | 7737 | 7754 | TTACCACTGACATATGGT | 42 | 182 |
| 1263118 | 162039 | 162056 | 7810 | 7827 | AAGCTGTTAAAGTGCTGC | 53 | 183 |
| 1263124 | 162093 | 162110 | 7864 | 7881 | TACAGTCTGGCTATATAC | 118 | 184 |
| 1263130 | 162099 | 162116 | 7870 | 7887 | TGTCTGTACAGTCTGGCT | 34 | 185 |
| 1263136 | 162130 | 162147 | 7901 | 7918 | TAATAGGTTAAGCAGTGT | 129 | 186 |
| 1263142 | 162250 | 162267 | 8021 | 8038 | GATTGTGATATCAACCTG | 53 | 187 |
| 1263148 | 162405 | 162422 | 8176 | 8193 | GTGAACCTATTTTGCTCC | 57 | 188 |
| 1263154 | 162483 | 162500 | 8254 | 8271 | TTAGTGCAGGTACTACCA | 72 | 189 |
| 1263160 | 162489 | 162506 | 8260 | 8277 | ATTCAGTTAGTGCAGGTA | 130 | 190 |
| 1263166 | 162510 | 162527 | 8281 | 8298 | ATAACATAAGCACTACCT | 75 | 191 |
| 1263172 | 162579 | 162596 | 8350 | 8367 | GAACAATCTAGAGCAGCA | 76 | 192 |
| 1263178 | 162641 | 162658 | 8412 | 8429 | CTATATAAGTTGACTACT | 81 | 193 |
| 1263184 | 162657 | 162674 | 8428 | 8445 | GTCCTGATGTAATTGACT | 78 | 194 |
| 1263190 | 162729 | 162746 | 8500 | 8517 | CGAATACAGTTTATCTAA | 122 | 195 |
| 1263196 | 162736 | 162753 | 8507 | 8524 | CAGTTCACGAATACAGTT | 103 | 196 |
| 1263202 | 162742 | 162759 | 8513 | 8530 | AGCATGCAGTTCACGAAT | 98 | 197 |

TABLE 3
Effect of modified oligonucleotides on amount of human SCNIA RNA
in HepG2 cells

| | | SEQ | | | | | |
|---|---|---|---|---|---|---|---|
| | SEQ ID | ID | SEQ | SEQ | | | |
| | NO: 1 | NO: 1 | ID NO: | ID NO: | | SCNIA | SEQ |
| Compound | Start | Stop | 2 Start | 2 Stop | | (% | ID |
| Number | Site | Site | Site | Site | Sequence (5′ to 3′) | UTC) | No. |
| 1262741 | 2871 | 2888 | N/A | N/A | GGAACAGTTCCATGAGTT | 99 | 198 |
| 1262747 | 2877 | 2894 | N/A | N/A | TCTGGAGGAACAGTTCCA | 28 | 199 |
| 1262753 | 2883 | 2900 | N/A | N/A | TGTTAATCTGGAGGAACA | 60 | 200 |
| 1262759 | 2889 | 2906 | N/A | N/A | CTGAAGTGTTAATCTGGA | 78 | 201 |
| 1262765 | 2895 | 2912 | N/A | N/A | TAACCCCTGAAGTGTTAA | 119 | 202 |
| 1262771 | 2901 | 2918 | N/A | N/A | CTTCCATAACCCCTGAAG | 61 | 203 |
| 1262777 | 2907 | 2924 | N/A | N/A | CTCCAGCTTCCATAACCC | 147 | 204 |
| 1262783 | 2920 | 2937 | N/A | N/A | AAAGCTCAGCTTCCTCCA | 56 | 205 |
| 1262789 | 2927 | 2944 | N/A | N/A | TGTAGTAAAAGCTCAGCT | 125 | 206 |
| 1262795 | 2936 | 2953 | N/A | N/A | CCCAAAAGATGTAGTAAA | 56 | 207 |
| 1262801 | 78351 | 78368 | 469 | 486 | GCACATTTTAATTACCAT | 83 | 208 |
| 1262807 | 78361 | 78378 | 479 | 496 | TTGTCATCCTGCACATTT | 92 | 209 |
| 1262813 | 160820 | 160837 | 6591 | 6608 | AAAGGAGTCCTGTTGATA | 76 | 210 |
| 1262819 | 160829 | 160846 | 6600 | 6617 | TGACCTCCTAAAGGAGTC | 115 | 211 |
| 1262825 | 160841 | 160858 | 6612 | 6629 | TCAGTTTGGCATTGACCT | 60 | 212 |
| 1262831 | 160880 | 160897 | 6651 | 6668 | TTGTAGGCACTGACCTTA | 45 | 213 |
| 1262837 | 160886 | 160903 | 6657 | 6674 | GTCTTATTGTAGGCACTG | 62 | 214 |
| 1262843 | 160950 | 160967 | 6721 | 6738 | GTAACCTCCTGTCAAGGT | 60 | 215 |
| 1262849 | 160957 | 160974 | 6728 | 6745 | GAGAACAGTAACCTCCTG | 61 | 216 |
| 1262855 | 160970 | 160987 | 6741 | 6758 | GTCAGCTGGTAGTGAGAA | 104 | 217 |
| 1262861 | 160996 | 161013 | 6767 | 6784 | CCATTGTGCATCTTATCT | 66 | 218 |
| 1262867 | 161004 | 161021 | 6775 | 6792 | CTGACTAGCCATTGTGCA | 48 | 219 |
| 1262873 | 161010 | 161027 | 6781 | 6798 | TACAGTCTGACTAGCCAT | 57 | 220 |
| 1262879 | 161016 | 161033 | 6787 | 6804 | GGTCCCTACAGTCTGACT | 92 | 221 |
| 1262885 | 161022 | 161039 | 6793 | 6810 | GAAACTGGTCCCTACAGT | 64 | 222 |
| 1262891 | 161033 | 161050 | 6804 | 6821 | TTGCACCCCTTGAAACTG | 108 | 223 |
| 1262897 | 161039 | 161056 | 6810 | 6827 | ACAGGTTTGCACCCCTTG | 74 | 224 |
| 1262903 | 161089 | 161106 | 6860 | 6877 | TGGATACAATTACTACAC | 58 | 225 |
| 1262909 | 161227 | 161244 | 6998 | 7015 | GGTCCTTAGCCTATTTCT | 59 | 226 |
| 1262915 | 161233 | 161250 | 7004 | 7021 | TATAGAGGTCCTTAGCCT | 32 | 227 |
| 1262921 | 161243 | 161260 | 7014 | 7031 | GCATACCTGTTATAGAGG | 107 | 228 |
| 1262927 | 161254 | 161271 | 7025 | 7042 | CCCCCCAGGTGGCATACC | 46 | 229 |
| 1262933 | 161260 | 161277 | 7031 | 7048 | GCCATACCCCCCAGGTGG | 66 | 230 |
| 1262939 | 161266 | 161283 | 7037 | 7054 | GTGGTTGCCATACCCCCC | 65 | 231 |
| 1262945 | 161272 | 161289 | 7043 | 7060 | GGCCATGTGGTTGCCATA | 53 | 232 |
| 1262951 | 161295 | 161312 | 7066 | 7083 | CCACGACTTTGTGTAGCT | 88 | 233 |
| 1262957 | 161301 | 161318 | 7072 | 7089 | TGCAAACCACGACTTTGT | 57 | 234 |
| 1262963 | 161307 | 161324 | 7078 | 7095 | CCCTCATGCAAACCACGA | 61 | 235 |
| 1262969 | 161315 | 161332 | 7086 | 7103 | GCAGCATGCCCTCATGCA | 80 | 236 |
| 1262975 | 161321 | 161338 | 7092 | 7109 | CTAAGTGCAGCATGCCCT | 54 | 237 |
| 1262981 | 161333 | 161350 | 7104 | 7121 | CATGCATGATCTCTAAGT | 45 | 238 |
| 1262987 | 161402 | 161419 | 7173 | 7190 | TTATCACCCAATTACCCC | 75 | 239 |
| 1262993 | 161408 | 161425 | 7179 | 7196 | CTCCACTTATCACCCAAT | 107 | 240 |
| 1262999 | 161415 | 161432 | 7186 | 7203 | AAAGCACCTCCACTTATC | 113 | 241 |
| 1263005 | 161442 | 161459 | 7213 | 7230 | GGCTGGATTTCGCAAAAC | 96 | 242 |
| 1263011 | 161473 | 161490 | 7244 | 7261 | GCCTACCCACAAATAATC | 114 | 243 |
| 1263017 | 161479 | 161496 | 7250 | 7267 | TTACTGGCCTACCCACAA | 72 | 244 |
| 1263023 | 161485 | 161502 | 7256 | 7273 | TAAGATTTACTGGCCTAC | 53 | 245 |
| 1263029 | 161497 | 161514 | 7268 | 7285 | GTTTGCACCTGCTAAGAT | 46 | 246 |
| 1263035 | 161503 | 161520 | 7274 | 7291 | AATGAAGTTTGCACCTGC | 55 | 247 |
| 1263041 | 161603 | 161620 | 7374 | 7391 | CAGTCTTCTGGCGGTGGA | 58 | 248 |
| 1263047 | 161611 | 161628 | 7382 | 7399 | GGTCAATTCAGTCTTCTG | 76 | 249 |
| 1263053 | 161669 | 161686 | 7440 | 7457 | AGCCGAAGATGGCTAAAC | 35 | 250 |
| 1263059 | 161675 | 161692 | 7446 | 7463 | GCTGAGAGCCGAAGATGG | 56 | 251 |
| 1263065 | 161682 | 161699 | 7453 | 7470 | CAACCTTGCTGAGAGCCG | 36 | 252 |
| 1263071 | 161692 | 161709 | 7463 | 7480 | TATACAGTGTCAACCTTG | 44 | 253 |
| 1263077 | 161745 | 161762 | 7516 | 7533 | GTGCACCACAGGGTAAAA | 69 | 254 |
| 1263083 | 161781 | 161798 | 7552 | 7569 | AATACTGTGCTTAGGTCA | 79 | 255 |
| 1263089 | 161830 | 161847 | 7601 | 7618 | GTGTAAAGCTTGCACTCT | 63 | 256 |
| 1263095 | 161875 | 161892 | 7646 | 7663 | GCATCCAAACTATCTATA | 84 | 257 |
| 1263101 | 161881 | 161898 | 7652 | 7669 | TTGATAGCATCCAAACTA | 51 | 258 |
| 1263107 | 161891 | 161908 | 7662 | 7679 | TAAACATGCATTGATAGC | 68 | 259 |
| 1263113 | 161968 | 161985 | 7739 | 7756 | CTTTACCACTGACATATG | 162 | 260 |
| 1263119 | 162079 | 162096 | 7850 | 7867 | ATACCATATGTTATCCAC | 93 | 261 |
| 1263125 | 162094 | 162111 | 7865 | 7882 | GTACAGTCTGGCTATATA | 74 | 262 |
| 1263131 | 162101 | 162118 | 7872 | 7889 | CATGTCTGTACAGTCTGG | 76 | 263 |
| 1263137 | 162131 | 162148 | 7902 | 7919 | TTAATAGGTTAAGCAGTG | 115 | 264 |
| 1263143 | 162251 | 162268 | 8022 | 8039 | TGATTGTGATATCAACCT | 54 | 265 |
| 1263149 | 162406 | 162423 | 8177 | 8194 | CGTGAACCTATTTTGCTC | 62 | 266 |
| 1263155 | 162484 | 162501 | 8255 | 8272 | GTTAGTGCAGGTACTACC | 48 | 267 |
| 1263161 | 162490 | 162507 | 8261 | 8278 | AATTCAGTTAGTGCAGGT | 51 | 268 |
| 1263167 | 162541 | 162558 | 8312 | 8329 | CATAAACCGAAGTCAGAA | 63 | 269 |
| 1263173 | 162583 | 162600 | 8354 | 8371 | TTTAGAACAATCTAGAGC | 76 | 270 |
| 1263179 | 162642 | 162659 | 8413 | 8430 | ACTATATAAGTTGACTAC | 47 | 271 |
| 1263185 | 162692 | 162709 | 8463 | 8480 | GAGCCTATGGTTTGCTTC | 83 | 272 |
| 1263191 | 162730 | 162747 | 8501 | 8518 | ACGAATACAGTTTATCTA | 124 | 273 |
| 1263197 | 162737 | 162754 | 8508 | 8525 | GCAGTTCACGAATACAGT | 83 | 274 |
| 1263203 | 162743 | 162760 | 8514 | 8531 | CAGCATGCAGTTCACGAA | 79 | 275 |

TABLE 4
Effect of modified oligonucleotides on amount of human SCNIA RNA
in HepG2 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | SCNIA | SEQ |
| Compound | Start | Stop | Start | Stop | | (% | ID |
| Number | Site | Site | Site | Site | Sequence (5′ to 3′) | UTC) | No. |
| 1262742 | 2872 | 2889 | N/A | N/A | AGGAACAGTTCCATGAGT | 38 | 276 |
| 1262748 | 2878 | 2895 | N/A | N/A | ATCTGGAGGAACAGTTCC | 48 | 277 |
| 1262754 | 2884 | 2901 | N/A | N/A | GTGTTAATCTGGAGGAAC | 69 | 278 |
| 1262760 | 2890 | 2907 | N/A | N/A | CCTGAAGTGTTAATCTGG | 37 | 279 |
| 1262766 | 2896 | 2913 | N/A | N/A | ATAACCCCTGAAGTGTTA | 63 | 280 |
| 1262772 | 2902 | 2919 | N/A | N/A | GCTTCCATAACCCCTGAA | 58 | 281 |
| 1262778 | 2908 | 2925 | N/A | N/A | CCTCCAGCTTCCATAACC | 68 | 282 |
| 1262784 | 2921 | 2938 | N/A | N/A | AAAAGCTCAGCTTCCTCC | 55 | 283 |
| 1262790 | 2928 | 2945 | N/A | N/A | ATGTAGTAAAAGCTCAGC | 75 | 284 |
| 1262796 | 2937 | 2954 | N/A | N/A | CCCCAAAAGATGTAGTAA | 76 | 285 |
| 1262802 | 78353 | 78370 | 471 | 488 | CTGCACATTTTAATTACC | 80 | 286 |
| 1262808 | 78362 | 78379 | 480 | 497 | CTTGTCATCCTGCACATT | 48 | 287 |
| 1262814 | 160821 | 160838 | 6592 | 6609 | TAAAGGAGTCCTGTTGAT | 56 | 288 |
| 1262820 | 160836 | 160853 | 6607 | 6624 | TTGGCATTGACCTCCTAA | 79 | 289 |
| 1262826 | 160842 | 160859 | 6613 | 6630 | GTCAGTTTGGCATTGACC | 54 | 290 |
| 1262832 | 160881 | 160898 | 6652 | 6669 | ATTGTAGGCACTGACCTT | 43 | 291 |
| 1262838 | 160887 | 160904 | 6658 | 6675 | TGTCTTATTGTAGGCACT | 69 | 292 |
| 1262844 | 160951 | 160968 | 6722 | 6739 | AGTAACCTCCTGTCAAGG | 70 | 293 |
| 1262850 | 160959 | 160976 | 6730 | 6747 | GTGAGAACAGTAACCTCC | 72 | 294 |
| 1262856 | 160971 | 160988 | 6742 | 6759 | TGTCAGCTGGTAGTGAGA | 71 | 295 |
| 1262862 | 160997 | 161014 | 6768 | 6785 | GCCATTGTGCATCTTATC | 40 | 296 |
| 1262868 | 161005 | 161022 | 6776 | 6793 | TCTGACTAGCCATTGTGC | 54 | 297 |
| 1262874 | 161011 | 161028 | 6782 | 6799 | CTACAGTCTGACTAGCCA | 72 | 298 |
| 1262880 | 161017 | 161034 | 6788 | 6805 | TGGTCCCTACAGTCTGAC | 66 | 299 |
| 1262886 | 161028 | 161045 | 6799 | 6816 | CCCCTTGAAACTGGTCCC | 52 | 300 |
| 1262892 | 161034 | 161051 | 6805 | 6822 | TTTGCACCCCTTGAAACT | 75 | 301 |
| 1262898 | 161040 | 161057 | 6811 | 6828 | CACAGGTTTGCACCCCTT | 57 | 302 |
| 1262904 | 161090 | 161107 | 6861 | 6878 | GTGGATACAATTACTACA | 42 | 303 |
| 1262910 | 161228 | 161245 | 6999 | 7016 | AGGTCCTTAGCCTATTTC | 44 | 304 |
| 1262916 | 161238 | 161255 | 7009 | 7026 | CCTGTTATAGAGGTCCTT | 41 | 305 |
| 1262922 | 161244 | 161261 | 7015 | 7032 | GGCATACCTGTTATAGAG | 86 | 306 |
| 1262928 | 161255 | 161272 | 7026 | 7043 | ACCCCCCAGGTGGCATAC | 56 | 307 |
| 1262934 | 161261 | 161278 | 7032 | 7049 | TGCCATACCCCCCAGGTG | 45 | 308 |
| 1262940 | 161267 | 161284 | 7038 | 7055 | TGTGGTTGCCATACCCCC | 66 | 309 |
| 1262946 | 161275 | 161292 | 7046 | 7063 | GAGGGCCATGTGGTTGCC | 55 | 310 |
| 1262952 | 161296 | 161313 | 7067 | 7084 | ACCACGACTTTGTGTAGC | 40 | 311 |
| 1262958 | 161302 | 161319 | 7073 | 7090 | ATGCAAACCACGACTTTG | 67 | 312 |
| 1262964 | 161308 | 161325 | 7079 | 7096 | GCCCTCATGCAAACCACG | 63 | 313 |
| 1262970 | 161316 | 161333 | 7087 | 7104 | TGCAGCATGCCCTCATGC | 46 | 314 |
| 1262976 | 161322 | 161339 | 7093 | 7110 | TCTAAGTGCAGCATGCCC | 83 | 315 |
| 1262982 | 161334 | 161351 | 7105 | 7122 | TCATGCATGATCTCTAAG | 63 | 316 |
| 1262988 | 161403 | 161420 | 7174 | 7191 | CTTATCACCCAATTACCC | 61 | 317 |
| 1262994 | 161409 | 161426 | 7180 | 7197 | CCTCCACTTATCACCCAA | 57 | 318 |
| 1263000 | 161435 | 161452 | 7206 | 7223 | TTTCGCAAAACAAGATCA | 48 | 319 |
| 1263006 | 161443 | 161460 | 7214 | 7231 | GGGCTGGATTTCGCAAAA | 87 | 320 |
| 1263012 | 161474 | 161491 | 7245 | 7262 | GGCCTACCCACAAATAAT | 66 | 321 |
| 1263018 | 161480 | 161497 | 7251 | 7268 | TTTACTGGCCTACCCACA | 44 | 322 |
| 1263024 | 161488 | 161505 | 7259 | 7276 | TGCTAAGATTTACTGGCC | 67 | 323 |
| 1263030 | 161498 | 161515 | 7269 | 7286 | AGTTTGCACCTGCTAAGA | 55 | 324 |
| 1263036 | 161504 | 161521 | 7275 | 7292 | GAATGAAGTTTGCACCTG | 77 | 325 |
| 1263042 | 161604 | 161621 | 7375 | 7392 | TCAGTCTTCTGGCGGTGG | 81 | 326 |
| 1263048 | 161659 | 161676 | 7430 | 7447 | GGCTAAACAAAGTGCAGG | 57 | 327 |
| 1263054 | 161670 | 161687 | 7441 | 7458 | GAGCCGAAGATGGCTAAA | 56 | 328 |
| 1263060 | 161677 | 161694 | 7448 | 7465 | TTGCTGAGAGCCGAAGAT | 66 | 329 |
| 1263066 | 161684 | 161701 | 7455 | 7472 | GTCAACCTTGCTGAGAGC | 59 | 330 |
| 1263072 | 161693 | 161710 | 7464 | 7481 | ATATACAGTGTCAACCTT | 77 | 331 |
| 1263078 | 161746 | 161763 | 7517 | 7534 | CGTGCACCACAGGGTAAA | 89 | 332 |
| 1263084 | 161782 | 161799 | 7553 | 7570 | AAATACTGTGCTTAGGTC | 50 | 333 |
| 1263090 | 161833 | 161850 | 7604 | 7621 | CCTGTGTAAAGCTTGCAC | 60 | 334 |
| 1263096 | 161876 | 161893 | 7647 | 7664 | AGCATCCAAACTATCTAT | 78 | 335 |
| 1263102 | 161883 | 161900 | 7654 | 7671 | CATTGATAGCATCCAAAC | 38 | 336 |
| 1263108 | 161907 | 161924 | 7678 | 7695 | CAGCAGCATGGTAATATA | 59 | 337 |
| 1263114 | 162026 | 162043 | 7797 | 7814 | GCTGCAAACTATTGCTTA | 49 | 338 |
| 1263120 | 162089 | 162106 | 7860 | 7877 | GTCTGGCTATATACCATA | 71 | 339 |
| 1263126 | 162095 | 162112 | 7866 | 7883 | TGTACAGTCTGGCTATAT | 64 | 340 |
| 1263132 | 162102 | 162119 | 7873 | 7890 | ACATGTCTGTACAGTCTG | 98 | 341 |
| 1263138 | 162244 | 162261 | 8015 | 8032 | GATATCAACCTGAAGATA | 52 | 342 |
| 1263144 | 162252 | 162269 | 8023 | 8040 | GTGATTGTGATATCAACC | 87 | 343 |
| 1263150 | 162478 | 162495 | 8249 | 8266 | GCAGGTACTACCAGAAAT | 91 | 344 |
| 1263156 | 162485 | 162502 | 8256 | 8273 | AGTTAGTGCAGGTACTAC | 47 | 345 |
| 1263162 | 162491 | 162508 | 8262 | 8279 | CAATTCAGTTAGTGCAGG | 43 | 346 |
| 1263168 | 162542 | 162559 | 8313 | 8330 | ACATAAACCGAAGTCAGA | 65 | 347 |
| 1263174 | 162596 | 162613 | 8367 | 8384 | AGCCCACATTCTATTTAG | 53 | 348 |
| 1263180 | 162643 | 162660 | 8414 | 8431 | GACTATATAAGTTGACTA | 35 | 349 |
| 1263186 | 162693 | 162710 | 8464 | 8481 | GGAGCCTATGGTTTGCTT | 67 | 350 |
| 1263192 | 162731 | 162748 | 8502 | 8519 | CACGAATACAGTTTATCT | 56 | 351 |
| 1263198 | 162738 | 162755 | 8509 | 8526 | TGCAGTTCACGAATACAG | 45 | 352 |
| 1263204 | 162744 | 162761 | 8515 | 8532 | CCAGCATGCAGTTCACGA | 61 | 353 |

TABLE 5
Effect of modified oligonucleotides on amount of human SCNIA RNA
in HepG2 cells

| | | SEQ | | | | | |
|---|---|---|---|---|---|---|---|
| | SEQ | ID | SEQ | SEQ | | | |
| | ID NO: | NO: 1 | ID NO: | ID NO: | | SCNIA | SEQ |
| Compound | 1 Start | Stop | 2 Start | 2 Stop | | (% | ID |
| Number | Site | Site | Site | Site | Sequence (5′ to 3′) | UTC) | No. |
| 1262743 | 2873 | 2890 | N/A | N/A | GAGGAACAGTTCCATGAG | 65 | 354 |
| 1262749 | 2879 | 2896 | N/A | N/A | AATCTGGAGGAACAGTTC | 58 | 355 |
| 1262755 | 2885 | 2902 | N/A | N/A | AGTGTTAATCTGGAGGAA | 47 | 356 |
| 1262761 | 2891 | 2908 | N/A | N/A | CCCTGAAGTGTTAATCTG | 68 | 357 |
| 1262767 | 2897 | 2914 | N/A | N/A | CATAACCCCTGAAGTGTT | 88 | 358 |
| 1262773 | 2903 | 2920 | N/A | N/A | AGCTTCCATAACCCCTGA | 36 | 359 |
| 1262779 | 2909 | 2926 | N/A | N/A | TCCTCCAGCTTCCATAAC | 52 | 360 |
| 1262785 | 2923 | 2940 | N/A | N/A | GTAAAAGCTCAGCTTCCT | 117 | 361 |
| 1262791 | 2929 | 2946 | N/A | N/A | GATGTAGTAAAAGCTCAG | 84 | 362 |
| 1262797 | 78337 | 78354 | 455 | 472 | CCATTTATTCTGCATATG | 111 | 363 |
| 1262803 | 78354 | 78371 | 472 | 489 | CCTGCACATTTTAATTAC | 84 | 364 |
| 1262809 | 160783 | 160800 | 6554 | 6571 | GGCTGTAAACAATTTGTC | 106 | 365 |
| 1262815 | 160822 | 160839 | 6593 | 6610 | CTAAAGGAGTCCTGTTGA | 122 | 366 |
| 1262821 | 160837 | 160854 | 6608 | 6625 | TTTGGCATTGACCTCCTA | 85 | 367 |
| 1262827 | 160843 | 160860 | 6614 | 6631 | AGTCAGTTTGGCATTGAC | 88 | 368 |
| 1262833 | 160882 | 160899 | 6653 | 6670 | TATTGTAGGCACTGACCT | 67 | 369 |
| 1262839 | 160888 | 160905 | 6659 | 6676 | CTGTCTTATTGTAGGCAC | 45 | 370 |
| 1262845 | 160952 | 160969 | 6723 | 6740 | CAGTAACCTCCTGTCAAG | 80 | 371 |
| 1262851 | 160960 | 160977 | 6731 | 6748 | AGTGAGAACAGTAACCTC | 47 | 372 |
| 1262857 | 160972 | 160989 | 6743 | 6760 | GTGTCAGCTGGTAGTGAG | 69 | 373 |
| 1262863 | 160998 | 161015 | 6769 | 6786 | AGCCATTGTGCATCTTAT | 127 | 374 |
| 1262869 | 161006 | 161023 | 6777 | 6794 | GTCTGACTAGCCATTGTG | 63 | 375 |
| 1262875 | 161012 | 161029 | 6783 | 6800 | CCTACAGTCTGACTAGCC | 104 | 376 |
| 1262881 | 161018 | 161035 | 6789 | 6806 | CTGGTCCCTACAGTCTGA | 97 | 377 |
| 1262887 | 161029 | 161046 | 6800 | 6817 | ACCCCTTGAAACTGGTCC | 72 | 378 |
| 1262893 | 161035 | 161052 | 6806 | 6823 | GTTTGCACCCCTTGAAAC | 63 | 379 |
| 1262899 | 161041 | 161058 | 6812 | 6829 | TCACAGGTTTGCACCCCT | 56 | 380 |
| 1262905 | 161091 | 161108 | 6862 | 6879 | AGTGGATACAATTACTAC | 67 | 381 |
| 1262911 | 161229 | 161246 | 7000 | 7017 | GAGGTCCTTAGCCTATTT | 78 | 382 |
| 1262917 | 161239 | 161256 | 7010 | 7027 | ACCTGTTATAGAGGTCCT | 69 | 383 |
| 1262923 | 161245 | 161262 | 7016 | 7033 | TGGCATACCTGTTATAGA | 75 | 384 |
| 1262929 | 161256 | 161273 | 7027 | 7044 | TACCCCCCAGGTGGCATA | 94 | 385 |
| 1262935 | 161262 | 161279 | 7033 | 7050 | TTGCCATACCCCCCAGGT | 105 | 386 |
| 1262941 | 161268 | 161285 | 7039 | 7056 | ATGTGGTTGCCATACCCC | 64 | 387 |
| 1262947 | 161289 | 161306 | 7060 | 7077 | CTTTGTGTAGCTGGGAGG | 61 | 388 |
| 1262953 | 161297 | 161314 | 7068 | 7085 | AACCACGACTTTGTGTAG | 79 | 389 |
| 1262959 | 161303 | 161320 | 7074 | 7091 | CATGCAAACCACGACTTT | 99 | 390 |
| 1262965 | 161310 | 161327 | 7081 | 7098 | ATGCCCTCATGCAAACCA | 61 | 391 |
| 1262971 | 161317 | 161334 | 7088 | 7105 | GTGCAGCATGCCCTCATG | 63 | 392 |
| 1262977 | 161323 | 161340 | 7094 | 7111 | CTCTAAGTGCAGCATGCC | 69 | 393 |
| 1262983 | 161335 | 161352 | 7106 | 7123 | CTCATGCATGATCTCTAA | 70 | 394 |
| 1262989 | 161404 | 161421 | 7175 | 7192 | ACTTATCACCCAATTACC | 70 | 395 |
| 1262995 | 161410 | 161427 | 7181 | 7198 | ACCTCCACTTATCACCCA | 63 | 396 |
| 1263001 | 161437 | 161454 | 7208 | 7225 | GATTTCGCAAAACAAGAT | 63 | 397 |
| 1263007 | 161459 | 161476 | 7230 | 7247 | AATCTACTTGGTCTAGGG | 87 | 398 |
| 1263013 | 161475 | 161492 | 7246 | 7263 | TGGCCTACCCACAAATAA | 58 | 399 |
| 1263019 | 161481 | 161498 | 7252 | 7269 | ATTTACTGGCCTACCCAC | 139 | 400 |
| 1263025 | 161493 | 161510 | 7264 | 7281 | GCACCTGCTAAGATTTAC | 84 | 401 |
| 1263031 | 161499 | 161516 | 7270 | 7287 | AAGTTTGCACCTGCTAAG | 82 | 402 |
| 1263037 | 161530 | 161547 | 7301 | 7318 | CATAACATTTATGACTCC | 78 | 403 |
| 1263043 | 161605 | 161622 | 7376 | 7393 | TTCAGTCTTCTGGCGGTG | 58 | 404 |
| 1263049 | 161662 | 161679 | 7433 | 7450 | GATGGCTAAACAAAGTGC | 67 | 405 |
| 1263055 | 161671 | 161688 | 7442 | 7459 | AGAGCCGAAGATGGCTAA | 68 | 406 |
| 1263061 | 161678 | 161695 | 7449 | 7466 | CTTGCTGAGAGCCGAAGA | 87 | 407 |
| 1263067 | 161687 | 161704 | 7458 | 7475 | AGTGTCAACCTTGCTGAG | 58 | 408 |
| 1263073 | 161694 | 161711 | 7465 | 7482 | CATATACAGTGTCAACCT | 73 | 409 |
| 1263079 | 161777 | 161794 | 7548 | 7565 | CTGTGCTTAGGTCATTAT | 76 | 410 |
| 1263085 | 161825 | 161842 | 7596 | 7613 | AAGCTTGCACTCTACATT | 78 | 411 |
| 1263091 | 161834 | 161851 | 7605 | 7622 | ACCTGTGTAAAGCTTGCA | 97 | 412 |
| 1263097 | 161877 | 161894 | 7648 | 7665 | TAGCATCCAAACTATCTA | 72 | 413 |
| 1263103 | 161884 | 161901 | 7655 | 7672 | GCATTGATAGCATCCAAA | 50 | 414 |
| 1263109 | 161911 | 161928 | 7682 | 7699 | GATACAGCAGCATGGTAA | 64 | 415 |
| 1263115 | 162028 | 162045 | 7799 | 7816 | GTGCTGCAAACTATTGCT | 68 | 416 |
| 1263121 | 162090 | 162107 | 7861 | 7878 | AGTCTGGCTATATACCAT | 100 | 417 |
| 1263127 | 162096 | 162113 | 7867 | 7884 | CTGTACAGTCTGGCTATA | 67 | 418 |
| 1263133 | 162103 | 162120 | 7874 | 7891 | AACATGTCTGTACAGTCT | 98 | 419 |
| 1263139 | 162246 | 162263 | 8017 | 8034 | GTGATATCAACCTGAAGA | 107 | 420 |
| 1263145 | 162253 | 162270 | 8024 | 8041 | AGTGATTGTGATATCAAC | 92 | 421 |
| 1263151 | 162480 | 162497 | 8251 | 8268 | GTGCAGGTACTACCAGAA | 67 | 422 |
| 1263157 | 162486 | 162503 | 8257 | 8274 | CAGTTAGTGCAGGTACTA | 88 | 423 |
| 1263163 | 162492 | 162509 | 8263 | 8280 | TCAATTCAGTTAGTGCAG | 42 | 424 |
| 1263169 | 162543 | 162560 | 8314 | 8331 | AACATAAACCGAAGTCAG | 60 | 425 |
| 1263175 | 162636 | 162653 | 8407 | 8424 | TAAGTTGACTACTCTGTT | 66 | 426 |
| 1263181 | 162645 | 162662 | 8416 | 8433 | TTGACTATATAAGTTGAC | 102 | 427 |
| 1263187 | 162694 | 162711 | 8465 | 8482 | AGGAGCCTATGGTTTGCT | 102 | 428 |
| 1263193 | 162732 | 162749 | 8503 | 8520 | TCACGAATACAGTTTATC | 74 | 429 |
| 1263199 | 162739 | 162756 | 8510 | 8527 | ATGCAGTTCACGAATACA | 44 | 430 |
| 1263205 | 162745 | 162762 | 8516 | 8533 | TCCAGCATGCAGTTCACG | 61 | 431 |

TABLE 6
Effect of modified oligonucleotides on amount of human SCNIA RNA
in HepG2 cells

| | | SEQ | | | | | |
|---|---|---|---|---|---|---|---|
| | SEQ | ID | SEQ | SEQ | | | |
| | ID NO: | NO: 1 | ID NO: | ID NO: | | SCNIA | SEQ |
| Compound | 1 Start | Stop | 2 Start | 2 Stop | | (% | ID |
| Number | Site | Site | Site | Site | Sequence (5′ to 3′) | UTC) | No. |
| 1262744 | 2874 | 2891 | N/A | N/A | GGAGGAACAGTTCCATGA | 33 | 432 |
| 1262750 | 2880 | 2897 | N/A | N/A | TAATCTGGAGGAACAGTT | 56 | 433 |
| 1262756 | 2886 | 2903 | N/A | N/A | AAGTGTTAATCTGGAGGA | 57 | 434 |
| 1262762 | 2892 | 2909 | N/A | N/A | CCCCTGAAGTGTTAATCT | 34 | 435 |
| 1262768 | 2898 | 2915 | N/A | N/A | CCATAACCCCTGAAGTGT | 57 | 436 |
| 1262774 | 2904 | 2921 | N/A | N/A | CAGCTTCCATAACCCCTG | 52 | 437 |
| 1262780 | 2910 | 2927 | N/A | N/A | TTCCTCCAGCTTCCATAA | 109 | 438 |
| 1262786 | 2924 | 2941 | N/A | N/A | AGTAAAAGCTCAGCTTCC | 53 | 439 |
| 1262792 | 2930 | 2947 | N/A | N/A | AGATGTAGTAAAAGCTCA | 57 | 440 |
| 1262798 | 78338 | 78355 | 456 | 473 | ACCATTTATTCTGCATAT | 43 | 441 |
| 1262804 | 78358 | 78375 | 476 | 493 | TCATCCTGCACATTTTAA | 77 | 442 |
| 1262810 | 160817 | 160834 | 6588 | 6605 | GGAGTCCTGTTGATAAAA | 111 | 443 |
| 1262816 | 160823 | 160840 | 6594 | 6611 | CCTAAAGGAGTCCTGTTG | 30 | 444 |
| 1262822 | 160838 | 160855 | 6609 | 6626 | GTTTGGCATTGACCTCCT | 62 | 445 |
| 1262828 | 160869 | 160886 | 6640 | 6657 | GACCTTAAGGAGATTTGT | 66 | 446 |
| 1262834 | 160883 | 160900 | 6654 | 6671 | TTATTGTAGGCACTGACC | 52 | 447 |
| 1262840 | 160889 | 160906 | 6660 | 6677 | ACTGTCTTATTGTAGGCA | 51 | 448 |
| 1262846 | 160953 | 160970 | 6724 | 6741 | ACAGTAACCTCCTGTCAA | 49 | 449 |
| 1262852 | 160961 | 160978 | 6732 | 6749 | TAGTGAGAACAGTAACCT | 97 | 450 |
| 1262858 | 160973 | 160990 | 6744 | 6761 | AGTGTCAGCTGGTAGTGA | 102 | 451 |
| 1262864 | 160999 | 161016 | 6770 | 6787 | TAGCCATTGTGCATCTTA | 79 | 452 |
| 1262870 | 161007 | 161024 | 6778 | 6795 | AGTCTGACTAGCCATTGT | 41 | 453 |
| 1262876 | 161013 | 161030 | 6784 | 6801 | CCCTACAGTCTGACTAGC | 170 | 454 |
| 1262882 | 161019 | 161036 | 6790 | 6807 | ACTGGTCCCTACAGTCTG | 40 | 455 |
| 1262888 | 161030 | 161047 | 6801 | 6818 | CACCCCTTGAAACTGGTC | 66 | 456 |
| 1262894 | 161036 | 161053 | 6807 | 6824 | GGTTTGCACCCCTTGAAA | 115 | 457 |
| 1262900 | 161043 | 161060 | 6814 | 6831 | AATCACAGGTTTGCACCC | 70 | 458 |
| 1262906 | 161146 | 161163 | 6917 | 6934 | AATCCACTAACAGATTCC | 123 | 459 |
| 1262912 | 161230 | 161247 | 7001 | 7018 | AGAGGTCCTTAGCCTATT | 44 | 460 |
| 1262918 | 161240 | 161257 | 7011 | 7028 | TACCTGTTATAGAGGTCC | 56 | 461 |
| 1262924 | 161246 | 161263 | 7017 | 7034 | GTGGCATACCTGTTATAG | 88 | 462 |
| 1262930 | 161257 | 161274 | 7028 | 7045 | ATACCCCCCAGGTGGCAT | 98 | 463 |
| 1262936 | 161263 | 161280 | 7034 | 7051 | GTTGCCATACCCCCCAGG | 54 | 464 |
| 1262942 | 161269 | 161286 | 7040 | 7057 | CATGTGGTTGCCATACCC | 45 | 465 |
| 1262948 | 161292 | 161309 | 7063 | 7080 | CGACTTTGTGTAGCTGGG | 35 | 466 |
| 1262954 | 161298 | 161315 | 7069 | 7086 | AAACCACGACTTTGTGTA | 93 | 467 |
| 1262960 | 161304 | 161321 | 7075 | 7092 | TCATGCAAACCACGACTT | 73 | 468 |
| 1262966 | 161312 | 161329 | 7083 | 7100 | GCATGCCCTCATGCAAAC | 43 | 469 |
| 1262972 | 161318 | 161335 | 7089 | 7106 | AGTGCAGCATGCCCTCAT | 76 | 470 |
| 1262978 | 161324 | 161341 | 7095 | 7112 | TCTCTAAGTGCAGCATGC | 110 | 471 |
| 1262984 | 161399 | 161416 | 7170 | 7187 | TCACCCAATTACCCCTCC | 66 | 472 |
| 1262990 | 161405 | 161422 | 7176 | 7193 | CACTTATCACCCAATTAC | 59 | 473 |
| 1262996 | 161411 | 161428 | 7182 | 7199 | CACCTCCACTTATCACCC | 69 | 474 |
| 1263002 | 161438 | 161455 | 7209 | 7226 | GGATTTCGCAAAACAAGA | 58 | 475 |
| 1263008 | 161460 | 161477 | 7231 | 7248 | TAATCTACTTGGTCTAGG | 42 | 476 |
| 1263014 | 161476 | 161493 | 7247 | 7264 | CTGGCCTACCCACAAATA | 104 | 477 |
| 1263020 | 161482 | 161499 | 7253 | 7270 | GATTTACTGGCCTACCCA | 35 | 478 |
| 1263026 | 161494 | 161511 | 7265 | 7282 | TGCACCTGCTAAGATTTA | 30 | 479 |
| 1263032 | 161500 | 161517 | 7271 | 7288 | GAAGTTTGCACCTGCTAA | 18 | 480 |
| 1263038 | 161599 | 161616 | 7370 | 7387 | CTTCTGGCGGTGGAGGGT | 97 | 481 |
| 1263044 | 161606 | 161623 | 7377 | 7394 | ATTCAGTCTTCTGGCGGT | 82 | 482 |
| 1263050 | 161666 | 161683 | 7437 | 7454 | CGAAGATGGCTAAACAAA | 66 | 483 |
| 1263056 | 161672 | 161689 | 7443 | 7460 | GAGAGCCGAAGATGGCTA | 58 | 484 |
| 1263062 | 161679 | 161696 | 7450 | 7467 | CCTTGCTGAGAGCCGAAG | 101 | 485 |
| 1263068 | 161689 | 161706 | 7460 | 7477 | ACAGTGTCAACCTTGCTG | 67 | 486 |
| 1263074 | 161695 | 161712 | 7466 | 7483 | ACATATACAGTGTCAACC | 76 | 487 |
| 1263080 | 161778 | 161795 | 7549 | 7566 | ACTGTGCTTAGGTCATTA | 75 | 488 |
| 1263086 | 161827 | 161844 | 7598 | 7615 | TAAAGCTTGCACTCTACA | 39 | 489 |
| 1263092 | 161835 | 161852 | 7606 | 7623 | TACCTGTGTAAAGCTTGC | 136 | 490 |
| 1263098 | 161878 | 161895 | 7649 | 7666 | ATAGCATCCAAACTATCT | 125 | 491 |
| 1263104 | 161885 | 161902 | 7656 | 7673 | TGCATTGATAGCATCCAA | 42 | 492 |
| 1263110 | 161912 | 161929 | 7683 | 7700 | AGATACAGCAGCATGGTA | 32 | 493 |
| 1263116 | 162029 | 162046 | 7800 | 7817 | AGTGCTGCAAACTATTGC | 67 | 494 |
| 1263122 | 162091 | 162108 | 7862 | 7879 | CAGTCTGGCTATATACCA | 55 | 495 |
| 1263128 | 162097 | 162114 | 7868 | 7885 | TCTGTACAGTCTGGCTAT | 128 | 496 |
| 1263134 | 162128 | 162145 | 7899 | 7916 | ATAGGTTAAGCAGTGTGT | 44 | 497 |
| 1263140 | 162247 | 162264 | 8018 | 8035 | TGTGATATCAACCTGAAG | 74 | 498 |
| 1263146 | 162336 | 162353 | 8107 | 8124 | ATCTACAACTACCCAGTC | 64 | 499 |
| 1263152 | 162481 | 162498 | 8252 | 8269 | AGTGCAGGTACTACCAGA | 76 | 500 |
| 1263158 | 162487 | 162504 | 8258 | 8275 | TCAGTTAGTGCAGGTACT | 60 | 501 |
| 1263164 | 162497 | 162514 | 8268 | 8285 | TACCTTCAATTCAGTTAG | 48 | 502 |
| 1263170 | 162572 | 162589 | 8343 | 8360 | CTAGAGCAGCATTACTCC | 42 | 503 |
| 1263176 | 162637 | 162654 | 8408 | 8425 | ATAAGTTGACTACTCTGT | 69 | 504 |
| 1263182 | 162655 | 162672 | 8426 | 8443 | CCTGATGTAATTGACTAT | 55 | 505 |
| 1263188 | 162695 | 162712 | 8466 | 8483 | GAGGAGCCTATGGTTTGC | 84 | 506 |
| 1263194 | 162734 | 162751 | 8505 | 8522 | GTTCACGAATACAGTTTA | 51 | 507 |
| 1263200 | 162740 | 162757 | 8511 | 8528 | CATGCAGTTCACGAATAC | 78 | 508 |
| 1263206 | 162767 | 162784 | 8538 | 8555 | ATTTAGCATAATAGTAGC | 78 | 509 |

### Example 2: Activity of Modified Oligonucleotides Targeting Human Scn1A in Hepg2 Cells, Single Dose, In Vitro

Modified oligonucleotides complementary to an SCN1A nucleic acid were synthesized and tested for their effect on SCN1A RNA levels in vitro. The modified oligonucleotides were tested in a series of experiments using the same culture conditions.

The modified oligonucleotides in the tables below are 18 nucleosides in length. Each nucleoside comprises a 2′-MOE sugar moiety. The internucleoside linkages throughout each modified oligonucleotide are phosphorothioate internucleoside linkages. All cytosine nucleobases throughout each modified oligonucleotide are 5-methylcytosines.

“Start site” indicates the 5′-most nucleoside of the target sequence to which the modified oligonucleotide is complementary. “Stop site” indicates the 3′-most nucleoside of the target sequence to which the modified oligonucleotide is complementary. As shown in the tables below, the modified oligonucleotides are complementary to either the human SCN1A genomic sequence, designated herein as SEQ ID NO: 1 (described herein above) or to the human SCN1A mRNA, designated herein as SEQ ID NO: 2 (described herein above) or to both. ‘N/A’ indicates that the modified oligonucleotide is not complementary to that particular target sequence with 100% complementarity.

Cultured HepG2 cells at a density of 20,000 cells per well were transfected using electroporation with 15,000 nM of modified oligonucleotide. After a treatment period of approximately 24 hours, RNA was isolated from the cells and SCN1A RNA levels were measured by quantitative real-time RTPCR. Human primer probe set RTS48189 (forward sequence CTATACCTCGACCAGGAAACAAA, designated herein as SEQ ID NO: 18; reverse sequence TGACCATGTTAAGACAGATGAGAA, designated herein as SEQ ID NO: 19; probe sequence TGTCTGGTTACGAAGTCAAAGACCATTCC, designated herein as SEQ ID NO: 20) was used to measure full-length SCN1A RNA levels. SCN1A RNA levels were normalized to total RNA content, as measured by RIBOGREEN®. SCN1A RNA is presented as % of the average of untreated control (% UTC). As shown in the tables below, certain modified oligonucleotides complementary to SCN1A RNA increased the amount of human SCN1A RNA compared to untreated control. Each of Tables 7-11 represents a different experiment.

TABLE 7
Effect of modified oligonucleotides on amount of human SCNIA RNA
in HepG2 cells

| | SEQ | SEQ | SEQ | SEQ | | | |
|---|---|---|---|---|---|---|---|
| | ID NO: | ID NO: | ID NO: | ID NO: | | SCN1A | SEQ |
| Compound | 1 Start | 1 Stop | 2 Start | 2 Stop | | (% | ID |
| Number | Site | Site | Site | Site | Sequence (5′ to 3′) | UTC) | No. |
| 1342105 | 144706 | 144723 | N/A | N/A | TTGGAGCAAGATTATCCT | 111 | 510 |
| 1342107 | 144766 | 144783 | N/A | N/A | GTAATACAGTACCCATAA | 61 | 511 |
| 1342109 | 144753 | 144770 | N/A | N/A | CATAATAAAGGGCTCAGG | 112 | 512 |
| 1342118 | 144686 | 144703 | N/A | N/A | ACAAAATAGAAATATATA | 173 | 513 |
| 1366952 | 95466 | 95483 | N/A | N/A | AACACACAAAAGAAAATC | 136 | 514 |
| 1366958 | 144722 | 144739 | N/A | N/A | CTCCACCCCATCCAAGTT | 114 | 515 |
| 1366964 | 152881 | 152898 | N/A | N/A | GAGCCTGAAAGAGGCTGA | 59 | 516 |
| 1366966 | 91106 | 91123 | N/A | N/A | GAAAAAGAAATATTAGGG | 89 | 517 |
| 1366970 | 91360 | 91377 | N/A | N/A | AAAGTGGGCACATCACCT | 57 | 518 |
| 1366971 | 144762 | 144779 | N/A | N/A | TACAGTACCCATAATAAA | 91 | 519 |
| 1366976 | 152950 | 152967 | N/A | N/A | AAAATACTACATCTTACA | 57 | 520 |
| 1366978 | 91136 | 91153 | N/A | N/A | TGATGAGAGCAAAACTCC | 76 | 521 |
| 1366981 | 110791 | 110808 | N/A | N/A | TTTGAAGACTAAACACAT | 82 | 522 |
| 1366982 | 144710 | 144727 | N/A | N/A | CAAGTTGGAGCAAGATTA | 138 | 523 |
| 1366983 | 110807 | 110824 | N/A | N/A | TTTTTCAAGCAGAAAATT | 66 | 524 |
| 1366988 | 152876 | 152893 | N/A | N/A | TGAAAGAGGCTGAAATCA | 73 | 525 |
| 1366991 | 152924 | 152941 | N/A | N/A | AAGAGCAAAGTTGGAATG | 118 | 526 |
| 1367000 | 110787 | 110804 | N/A | N/A | AAGACTAAACACATTTAC | 83 | 527 |
| 1367003 | 95470 | 95487 | N/A | N/A | AAGGAACACACAAAAGAA | 54 | 528 |
| 1367004 | 144682 | 144699 | N/A | N/A | AATAGAAATATATAGTTT | 85 | 529 |
| 1367005 | 95434 | 95451 | N/A | N/A | ATATTACAAAAAGCTAAC | 55 | 530 |
| 1367011 | 110827 | 110844 | N/A | N/A | ACACAATTAAATGTAAAC | 167 | 531 |
| 1367016 | 110823 | 110840 | N/A | N/A | AATTAAATGTAAACAGTT | 85 | 532 |
| 1367021 | 95450 | 95467 | N/A | N/A | TCAAAATCCAAGTGTTAT | 67 | 533 |
| 1367022 | 152955 | 152972 | N/A | N/A | GATAGAAAATACTACATC | 83 | 534 |
| 1367024 | 152941 | 152958 | N/A | N/A | CATCTTACAAAGTTTTGA | 53 | 535 |
| 1367035 | 152937 | 152954 | N/A | N/A | TTACAAAGTTTTGAAGAG | 66 | 536 |
| 1367038 | 152911 | 152928 | N/A | N/A | GAATGAGCATGAATTTCA | 68 | 537 |
| 1367045 | 95458 | 95475 | N/A | N/A | AAAGAAAATCAAAATCCA | 108 | 538 |
| 1367047 | 144758 | 144775 | N/A | N/A | GTACCCATAATAAAGGGC | 61 | 539 |
| 1367048 | 152885 | 152902 | N/A | N/A | CTTGGAGCCTGAAAGAGG | 74 | 540 |
| 1367049 | 110799 | 110816 | N/A | N/A | GCAGAAAATTTGAAGACT | 83 | 541 |
| 1367053 | 110819 | 110836 | N/A | N/A | AAATGTAAACAGTTTTTC | 89 | 542 |
| 1367066 | 152907 | 152924 | N/A | N/A | GAGCATGAATTTCAGTTT | 78 | 543 |
| 1367068 | 152959 | 152976 | N/A | N/A | GGTTGATAGAAAATACTA | 88 | 544 |
| 1367073 | 95454 | 95471 | N/A | N/A | AAAATCAAAATCCAAGTG | 113 | 545 |
| 1367084 | 144698 | 144715 | N/A | N/A | AGATTATCCTATACAAAA | 71 | 546 |
| 1367088 | 95446 | 95463 | N/A | N/A | AATCCAAGTGTTATATTA | 80 | 547 |
| 1367090 | 95462 | 95479 | N/A | N/A | CACAAAAGAAAATCAAAA | 69 | 548 |
| 1367097 | 95422 | 95439 | N/A | N/A | GCTAACATTGAAAAGCCC | 55 | 549 |
| 1367098 | 144714 | 144731 | N/A | N/A | CATCCAAGTTGGAGCAAG | 147 | 550 |
| 1367102 | 152895 | 152912 | N/A | N/A | CAGTTTAGGTCTTGGAGC | 76 | 551 |
| 1367103 | 110795 | 110812 | N/A | N/A | AAAATTTGAAGACTAAAC | 44 | 552 |
| 1367108 | 152903 | 152920 | N/A | N/A | ATGAATTTCAGTTTAGGT | 77 | 553 |
| 1367109 | 152899 | 152916 | N/A | N/A | ATTTCAGTTTAGGTCTTG | 69 | 554 |
| 1367113 | 152946 | 152963 | N/A | N/A | TACTACATCTTACAAAGT | 106 | 555 |
| 1367116 | 95438 | 95455 | N/A | N/A | TGTTATATTACAAAAAGC | 77 | 556 |
| 1367123 | 110803 | 110820 | N/A | N/A | TCAAGCAGAAAATTTGAA | 95 | 557 |
| 1367124 | 152928 | 152945 | N/A | N/A | TTTGAAGAGCAAAGTTGG | 75 | 558 |
| 1367127 | 91140 | 91157 | N/A | N/A | TGGGTGATGAGAGCAAAA | 111 | 559 |
| 1367140 | 110771 | 110788 | N/A | N/A | ACCTTCCAATATGCTTAC | 77 | 560 |
| 1367141 | 144690 | 144707 | N/A | N/A | CTATACAAAATAGAAATA | 94 | 561 |
| 1367145 | 91144 | 91161 | N/A | N/A | AGCCTGGGTGATGAGAGC | 69 | 562 |
| 1367146 | 152932 | 152949 | N/A | N/A | AAGTTTTGAAGAGCAAAG | 75 | 563 |
| 1367147 | 95478 | 95495 | N/A | N/A | TTATTGTTAAGGAACACA | 121 | 564 |
| 1367150 | 144694 | 144711 | N/A | N/A | TATCCTATACAAAATAGA | 57 | 565 |
| 1367151 | 110775 | 110792 | N/A | N/A | ATTTACCTTCCAATATGC | 77 | 566 |
| 1367153 | 152867 | 152884 | N/A | N/A | CTGAAATCAAATAAAATA | 104 | 567 |
| 1367162 | 95426 | 95443 | N/A | N/A | AAAAGCTAACATTGAAAA | 63 | 568 |
| 1367163 | 144702 | 144719 | N/A | N/A | AGCAAGATTATCCTATAC | 139 | 569 |
| 1367165 | 110783 | 110800 | N/A | N/A | CTAAACACATTTACCTTC | 117 | 570 |
| 1367166 | 110811 | 110828 | N/A | N/A | ACAGTTTTTCAAGCAGAA | 118 | 571 |
| 1367171 | 91216 | 91233 | N/A | N/A | AGGAGAACAGGAGAATCG | 83 | 572 |
| 1367185 | 95442 | 95459 | N/A | N/A | CAAGTGTTATATTACAAA | 85 | 573 |
| 1367187 | 95474 | 95491 | N/A | N/A | TGTTAAGGAACACACAAA | 50 | 574 |
| 1367189 | 144718 | 144735 | N/A | N/A | ACCCCATCCAAGTTGGAG | 82 | 575 |
| 1367192 | 152872 | 152889 | N/A | N/A | AGAGGCTGAAATCAAATA | 90 | 576 |
| 1367193 | 152919 | 152936 | N/A | N/A | CAAAGTTGGAATGAGCAT | 84 | 577 |
| 1367195 | 91220 | 91237 | N/A | N/A | AGGCAGGAGAACAGGAGA | 83 | 578 |
| 1367196 | 152890 | 152907 | N/A | N/A | TAGGTCTTGGAGCCTGAA | 69 | 579 |
| 1367200 | 152915 | 152932 | N/A | N/A | GTTGGAATGAGCATGAAT | 88 | 580 |
| 1367203 | 110815 | 110832 | N/A | N/A | GTAAACAGTTTTTCAAGC | 63 | 581 |
| 1367205 | 144678 | 144695 | N/A | N/A | GAAATATATAGTTTGTTA | 63 | 582 |
| 1367215 | 152856 | 152873 | N/A | N/A | TAAAATAGGTTTATATTT | 71 | 583 |
| 1367229 | 110779 | 110796 | N/A | N/A | ACACATTTACCTTCCAAT | 72 | 584 |
| 1367231 | 95430 | 95447 | N/A | N/A | TACAAAAAGCTAACATTG | 121 | 585 |
| 1367240 | 152861 | 152878 | N/A | N/A | TCAAATAAAATAGGTTTA | 123 | 586 |

TABLE 8
Effect of modified oligonucleotides on amount of human SCNIA
RNA in HepG2 cells

| | SEQ | SEQ | SEQ | SEQ | | | |
|---|---|---|---|---|---|---|---|
| | ID NO: | ID NO: | ID NO: | ID NO: | | SCNIA | SEQ |
| Compound | 1 Start | 1 Stop | 2 Start | 2 Stop | | (% | ID |
| Number | Site | Site | Site | Site | Sequence (5′ to 3′) | UTC) | No. |
| 1342110 | 144711 | 144728 | N/A | N/A | CCAAGTTGGAGCAAGATT | 67 | 587 |
| 1342115 | 144691 | 144708 | N/A | N/A | CCTATACAAAATAGAAAT | 25 | 588 |
| 1366953 | 95479 | 95496 | N/A | N/A | GTTATTGTTAAGGAACAC | 40 | 589 |
| 1366954 | 144703 | 144720 | N/A | N/A | GAGCAAGATTATCCTATA | 72 | 590 |
| 1366957 | 95447 | 95464 | N/A | N/A | AAATCCAAGTGTTATATT | 117 | 591 |
| 1366959 | 144699 | 144716 | N/A | N/A | AAGATTATCCTATACAAA | 54 | 592 |
| 1366967 | 91137 | 91154 | N/A | N/A | GTGATGAGAGCAAAACTC | 50 | 593 |
| 1366973 | 95455 | 95472 | N/A | N/A | GAAAATCAAAATCCAAGT | 43 | 594 |
| 1366974 | 152929 | 152946 | N/A | N/A | TTTTGAAGAGCAAAGTTG | 81 | 595 |
| 1366977 | 110828 | 110845 | N/A | N/A | TACACAATTAAATGTAAA | 213 | 596 |
| 1366980 | 152908 | 152925 | N/A | N/A | TGAGCATGAATTTCAGTT | 152 | 597 |
| 1366984 | 152904 | 152921 | N/A | N/A | CATGAATTTCAGTTTAGG | 124 | 598 |
| 1366990 | 95435 | 95452 | N/A | N/A | TATATTACAAAAAGCTAA | 62 | 599 |
| 1366992 | 110788 | 110805 | N/A | N/A | GAAGACTAAACACATTTA | 57 | 600 |
| 1366995 | 152878 | 152895 | N/A | N/A | CCTGAAAGAGGCTGAAAT | 66 | 601 |
| 1366999 | 91213 | 91230 | N/A | N/A | AGAACAGGAGAATCGCTT | 60 | 602 |
| 1367002 | 91217 | 91234 | N/A | N/A | CAGGAGAACAGGAGAATC | 44 | 603 |
| 1367014 | 152938 | 152955 | N/A | N/A | CTTACAAAGTTTTGAAGA | 155 | 604 |
| 1367015 | 152868 | 152885 | N/A | N/A | GCTGAAATCAAATAAAAT | 51 | 605 |
| 1367017 | 95443 | 95460 | N/A | N/A | CCAAGTGTTATATTACAA | 25 | 606 |
| 1367020 | 152896 | 152913 | N/A | N/A | TCAGTTTAGGTCTTGGAG | 109 | 607 |
| 1367023 | 110804 | 110821 | N/A | N/A | TTCAAGCAGAAAATTTGA | 62 | 608 |
| 1367031 | 152934 | 152951 | N/A | N/A | CAAAGTTTTGAAGAGCAA | 25 | 609 |
| 1367034 | 95451 | 95468 | N/A | N/A | ATCAAAATCCAAGTGTTA | 52 | 610 |
| 1367036 | 152947 | 152964 | N/A | N/A | ATACTACATCTTACAAAG | 85 | 611 |
| 1367050 | 95475 | 95492 | N/A | N/A | TTGTTAAGGAACACACAA | 24 | 612 |
| 1367054 | 91361 | 91378 | N/A | N/A | CAAAGTGGGCACATCACC | 69 | 613 |
| 1367056 | 152956 | 152973 | N/A | N/A | TGATAGAAAATACTACAT | 69 | 614 |
| 1367057 | 95463 | 95480 | N/A | N/A | ACACAAAAGAAAATCAAA | 63 | 615 |
| 1367058 | 152951 | 152968 | N/A | N/A | GAAAATACTACATCTTAC | 73 | 616 |
| 1367059 | 152920 | 152937 | N/A | N/A | GCAAAGTTGGAATGAGCA | 107 | 617 |
| 1367060 | 95459 | 95476 | N/A | N/A | AAAAGAAAATCAAAATCC | 44 | 618 |
| 1367061 | 144679 | 144696 | N/A | N/A | AGAAATATATAGTTTGTT | 56 | 619 |
| 1367069 | 152891 | 152908 | N/A | N/A | TTAGGTCTTGGAGCCTGA | 61 | 620 |
| 1367075 | 152882 | 152899 | N/A | N/A | GGAGCCTGAAAGAGGCTG | 62 | 621 |
| 1367078 | 144687 | 144704 | N/A | N/A | TACAAAATAGAAATATAT | 34 | 622 |
| 1367081 | 95471 | 95488 | N/A | N/A | TAAGGAACACACAAAAGA | 54 | 623 |
| 1367099 | 152887 | 152904 | N/A | N/A | GTCTTGGAGCCTGAAAGA | 96 | 624 |
| 1367107 | 144695 | 144712 | N/A | N/A | TTATCCTATACAAAATAG | 101 | 625 |
| 1367114 | 110792 | 110809 | N/A | N/A | ATTTGAAGACTAAACACA | 28 | 626 |
| 1367117 | 110780 | 110797 | N/A | N/A | AACACATTTACCTTCCAA | 45 | 627 |
| 1367121 | 144719 | 144736 | N/A | N/A | CACCCCATCCAAGTTGGA | 188 | 628 |
| 1367122 | 152925 | 152942 | N/A | N/A | GAAGAGCAAAGTTGGAAT | 56 | 629 |
| 1367125 | 91107 | 91124 | N/A | N/A | AGAAAAAGAAATATTAGG | 78 | 630 |
| 1367128 | 95427 | 95444 | N/A | N/A | AAAAAGCTAACATTGAAA | 65 | 631 |
| 1367129 | 110820 | 110837 | N/A | N/A | TAAATGTAAACAGTTTTT | 55 | 632 |
| 1367133 | 152858 | 152875 | N/A | N/A | AATAAAATAGGTTTATAT | 42 | 633 |
| 1367135 | 144759 | 144776 | N/A | N/A | AGTACCCATAATAAAGGG | 102 | 634 |
| 1367137 | 110816 | 110833 | N/A | N/A | TGTAAACAGTTTTTCAAG | 22 | 635 |
| 1367152 | 95423 | 95440 | N/A | N/A | AGCTAACATTGAAAAGCC | 46 | 636 |
| 1367155 | 144723 | 144740 | N/A | N/A | GCTCCACCCCATCCAAGT | 161 | 637 |
| 1367157 | 110784 | 110801 | N/A | N/A | ACTAAACACATTTACCTT | 82 | 638 |
| 1367161 | 152916 | 152933 | N/A | N/A | AGTTGGAATGAGCATGAA | 59 | 639 |
| 1367164 | 152900 | 152917 | N/A | N/A | AATTTCAGTTTAGGTCTT | 76 | 640 |
| 1367172 | 152912 | 152929 | N/A | N/A | GGAATGAGCATGAATITC | 73 | 641 |
| 1367174 | 144763 | 144780 | N/A | N/A | ATACAGTACCCATAATAA | 48 | 642 |
| 1367177 | 110776 | 110793 | N/A | N/A | CATTTACCTTCCAATATG | 28 | 643 |
| 1367179 | 95431 | 95448 | N/A | N/A | TTACAAAAAGCTAACATT | 69 | 644 |
| 1367182 | 144715 | 144732 | N/A | N/A | CCATCCAAGTTGGAGCAA | 129 | 645 |
| 1367184 | 144767 | 144784 | N/A | N/A | GGTAATACAGTACCCATA | 125 | 646 |
| 1367188 | 95439 | 95456 | N/A | N/A | GTGTTATATTACAAAAAG | 82 | 647 |
| 1367191 | 110772 | 110789 | N/A | N/A | TACCTTCCAATATGCTTA | 9 | 648 |
| 1367197 | 110796 | 110813 | N/A | N/A | GAAAATTTGAAGACTAAA | 38 | 649 |
| 1367208 | 152960 | 152977 | N/A | N/A | AGGTTGATAGAAAATACT | 36 | 650 |
| 1367210 | 144754 | 144771 | N/A | N/A | CCATAATAAAGGGCTCAG | 31 | 651 |
| 1367212 | 152873 | 152890 | N/A | N/A | AAGAGGCTGAAATCAAAT | 66 | 652 |
| 1367214 | 110824 | 110841 | N/A | N/A | CAATTAAATGTAAACAGT | 165 | 653 |
| 1367216 | 91141 | 91158 | N/A | N/A | CTGGGTGATGAGAGCAAA | 100 | 654 |
| 1367218 | 144707 | 144724 | N/A | N/A | GTTGGAGCAAGATTATCC | 112 | 655 |
| 1367221 | 144683 | 144700 | N/A | N/A | AAATAGAAATATATAGTT | 76 | 656 |
| 1367222 | 91221 | 91238 | N/A | N/A | GAGGCAGGAGAACAGGAG | 51 | 657 |
| 1367223 | 152862 | 152879 | N/A | N/A | ATCAAATAAAATAGGTTT | 70 | 658 |
| 1367228 | 110812 | 110829 | N/A | N/A | AACAGTTTTTCAAGCAGA | 72 | 659 |
| 1367232 | 95467 | 95484 | N/A | N/A | GAACACACAAAAGAAAAT | 141 | 660 |
| 1367233 | 110800 | 110817 | N/A | N/A | AGCAGAAAATTTGAAGAC | 52 | 661 |
| 1367236 | 110808 | 110825 | N/A | N/A | GTTTTTCAAGCAGAAAAT | 239 | 662 |
| 1367243 | 152942 | 152959 | N/A | N/A | ACATCTTACAAAGTTTTG | 41 | 663 |

TABLE 9
Effect of modified oligonucleotides on amount of human SCNIA RNA
in HepG2 cells

| | SEQ | SEQ | SEQ | SEQ | | | |
|---|---|---|---|---|---|---|---|
| | ID NO: | ID NO: | ID NO: | ID NO: | | SCN1A | SEQ |
| Compound | 1 Start | 1 Stop | 2 Start | 2 Stop | | (% | ID |
| Number | Site | Site | Site | Site | Sequence (5′ to 3′) | UTC) | No. |
| 1342108 | 144716 | 144733 | N/A | N/A | CCCATCCAAGTTGGAGCA | 97 | 664 |
| 1342111 | 144768 | 144785 | N/A | N/A | GGGTAATACAGTACCCAT | 50 | 665 |
| 1342122 | 144696 | 144713 | N/A | N/A | ATTATCCTATACAAAATA | 60 | 666 |
| 1366955 | 95456 | 95473 | N/A | N/A | AGAAAATCAAAATCCAAG | 41 | 667 |
| 1366961 | 152888 | 152905 | N/A | N/A | GGTCTTGGAGCCTGAAAG | 136 | 668 |
| 1366962 | 95468 | 95485 | N/A | N/A | GGAACACACAAAAGAAAA | 117 | 669 |
| 1366963 | 152952 | 152969 | N/A | N/A | AGAAAATACTACATCTTA | 55 | 670 |
| 1366965 | 152926 | 152943 | N/A | N/A | TGAAGAGCAAAGTTGGAA | 79 | 671 |
| 1366968 | 152865 | 152882 | N/A | N/A | GAAATCAAATAAAATAGG | 54 | 672 |
| 1366969 | 95452 | 95469 | N/A | N/A | AATCAAAATCCAAGTGTT | 54 | 673 |
| 1366972 | 95460 | 95477 | N/A | N/A | CAAAAGAAAATCAAAATC | 66 | 674 |
| 1366975 | 110829 | 110846 | N/A | N/A | ATACACAATTAAATGTAA | 60 | 675 |
| 1366985 | 95440 | 95457 | N/A | N/A | AGTGTTATATTACAAAAA | 52 | 676 |
| 1366996 | 152909 | 152926 | N/A | N/A | ATGAGCATGAATTTCAGT | 67 | 677 |
| 1366997 | 152917 | 152934 | N/A | N/A | AAGTTGGAATGAGCATGA | 71 | 678 |
| 1367001 | 95424 | 95441 | N/A | N/A | AAGCTAACATTGAAAAGC | 93 | 679 |
| 1367007 | 152905 | 152922 | N/A | N/A | GCATGAATTTCAGTTTAG | 68 | 680 |
| 1367008 | 152944 | 152961 | N/A | N/A | CTACATCTTACAAAGTTT | 104 | 681 |
| 1367018 | 152897 | 152914 | N/A | N/A | TTCAGTTTAGGTCTTGGA | 87 | 682 |
| 1367025 | 91218 | 91235 | N/A | N/A | GCAGGAGAACAGGAGAAT | 59 | 683 |
| 1367026 | 152874 | 152891 | N/A | N/A | AAAGAGGCTGAAATCAAA | 68 | 684 |
| 1367029 | 95420 | 95437 | N/A | N/A | TAACATTGAAAAGCCCAA | 78 | 685 |
| 1367032 | 91142 | 91159 | N/A | N/A | CCTGGGTGATGAGAGCAA | 98 | 686 |
| 1367033 | 144688 | 144705 | N/A | N/A | ATACAAAATAGAAATATA | 77 | 687 |
| 1367037 | 152901 | 152918 | N/A | N/A | GAATTTCAGTTTAGGTCT | 95 | 688 |
| 1367039 | 144755 | 144772 | N/A | N/A | CCCATAATAAAGGGCTCA | 66 | 689 |
| 1367040 | 152961 | 152978 | N/A | N/A | AAGGTTGATAGAAAATAC | 133 | 690 |
| 1367042 | 91222 | 91239 | N/A | N/A | TGAGGCAGGAGAACAGGA | 57 | 691 |
| 1367043 | 144712 | 144729 | N/A | N/A | TCCAAGTTGGAGCAAGAT | 67 | 692 |
| 1367044 | 144700 | 144717 | N/A | N/A | CAAGATTATCCTATACAA | 57 | 693 |
| 1367071 | 144760 | 144777 | N/A | N/A | CAGTACCCATAATAAAGG | 49 | 694 |
| 1367072 | 152869 | 152886 | N/A | N/A | GGCTGAAATCAAATAAAA | 84 | 695 |
| 1367076 | 144704 | 144721 | N/A | N/A | GGAGCAAGATTATCCTAT | 113 | 696 |
| 1367079 | 95428 | 95445 | N/A | N/A | CAAAAAGCTAACATTGAA | 38 | 697 |
| 1367080 | 110789 | 110806 | N/A | N/A | TGAAGACTAAACACATTT | 29 | 698 |
| 1367082 | 152957 | 152974 | N/A | N/A | TTGATAGAAAATACTACA | 89 | 699 |
| 1367083 | 152883 | 152900 | N/A | N/A | TGGAGCCTGAAAGAGGCT | 81 | 700 |
| 1367085 | 110793 | 110810 | N/A | N/A | AATTTGAAGACTAAACAC | 46 | 701 |
| 1367087 | 152930 | 152947 | N/A | N/A | GTTTTGAAGAGCAAAGTT | 85 | 702 |
| 1367089 | 144684 | 144701 | N/A | N/A | AAAATAGAAATATATAGT | 70 | 703 |
| 1367091 | 152892 | 152909 | N/A | N/A | TTTAGGTCTTGGAGCCTG | 34 | 704 |
| 1367093 | 95432 | 95449 | N/A | N/A | ATTACAAAAAGCTAACAT | 57 | 705 |
| 1367096 | 110813 | 110830 | N/A | N/A | AAACAGTTTTTCAAGCAG | 59 | 706 |
| 1367100 | 110773 | 110790 | N/A | N/A | TTACCTTCCAATATGCTT | 24 | 707 |
| 1367101 | 110801 | 110818 | N/A | N/A | AAGCAGAAAATTTGAAGA | 102 | 708 |
| 1367104 | 110825 | 110842 | N/A | N/A | ACAATTAAATGTAAACAG | 58 | 709 |
| 1367105 | 110805 | 110822 | N/A | N/A | TTTCAAGCAGAAAATTTG | 78 | 710 |
| 1367110 | 95480 | 95497 | N/A | N/A | GGTTATTGTTAAGGAACA | 61 | 711 |
| 1367115 | 95444 | 95461 | N/A | N/A | TCCAAGTGTTATATTACA | 72 | 712 |
| 1367119 | 95436 | 95453 | N/A | N/A | TTATATTACAAAAAGCTA | 99 | 713 |
| 1367120 | 110777 | 110794 | N/A | N/A | ACATTTACCTTCCAATAT | 79 | 714 |
| 1367126 | 95472 | 95489 | N/A | N/A | TTAAGGAACACACAAAAG | 39 | 715 |
| 1367131 | 144724 | 144741 | N/A | N/A | CGCTCCACCCCATCCAAG | 167 | 716 |
| 1367132 | 95464 | 95481 | N/A | N/A | CACACAAAAGAAAATCAA | 155 | 717 |
| 1367136 | 152913 | 152930 | N/A | N/A | TGGAATGAGCATGAATTT | 55 | 718 |
| 1367138 | 152939 | 152956 | N/A | N/A | TCTTACAAAGTTTTGAAG | 36 | 719 |
| 1367139 | 152948 | 152965 | N/A | N/A | AATACTACATCTTACAAA | 66 | 720 |
| 1367143 | 152921 | 152938 | N/A | N/A | AGCAAAGTTGGAATGAGC | 97 | 721 |
| 1367144 | 144720 | 144737 | N/A | N/A | CCACCCCATCCAAGTTGG | 45 | 722 |
| 1367167 | 110781 | 110798 | N/A | N/A | AAACACATTTACCTTCCA | 46 | 723 |
| 1367169 | 110821 | 110838 | N/A | N/A | TTAAATGTAAACAGTTTT | 61 | 724 |
| 1367173 | 91138 | 91155 | N/A | N/A | GGTGATGAGAGCAAAACT | 64 | 725 |
| 1367175 | 91108 | 91125 | N/A | N/A | AAGAAAAAGAAATATTAG | 32 | 726 |
| 1367176 | 110797 | 110814 | N/A | N/A | AGAAAATTTGAAGACTAA | 36 | 727 |
| 1367180 | 95476 | 95493 | N/A | N/A | ATTGTTAAGGAACACACA | 101 | 728 |
| 1367194 | 152879 | 152896 | N/A | N/A | GCCTGAAAGAGGCTGAAA | 58 | 729 |
| 1367198 | 91214 | 91231 | N/A | N/A | GAGAACAGGAGAATCGCT | 45 | 730 |
| 1367204 | 144692 | 144709 | N/A | N/A | TCCTATACAAAATAGAAA | 72 | 731 |
| 1367206 | 110817 | 110834 | N/A | N/A | ATGTAAACAGTTTTTCAA | 18 | 732 |
| 1367207 | 95448 | 95465 | N/A | N/A | AAAATCCAAGTGTTATAT | 107 | 733 |
| 1367209 | 110785 | 110802 | N/A | N/A | GACTAAACACATTTACCT | 97 | 734 |
| 1367211 | 144680 | 144697 | N/A | N/A | TAGAAATATATAGTTTGT | 157 | 735 |
| 1367219 | 144764 | 144781 | N/A | N/A | AATACAGTACCCATAATA | 49 | 736 |
| 1367220 | 110809 | 110826 | N/A | N/A | AGTTTTTCAAGCAGAAAA | 108 | 737 |
| 1367225 | 152935 | 152952 | N/A | N/A | ACAAAGTTTTGAAGAGCA | 82 | 738 |
| 1367227 | 152859 | 152876 | N/A | N/A | AAATAAAATAGGTTTATA | 92 | 739 |

TABLE 10
Effect of modified oligonucleotides on amount of human SCNIA RNA
in HepG2 cells

| | SEQ | SEQ | SEQ | SEQ | | | |
|---|---|---|---|---|---|---|---|
| | ID NO: | ID NO: | ID NO: | ID NO: | | SCNIA | SEQ |
| Compound | 1 Start | 1 Stop | 2 Start | 2 Stop | | (% | ID |
| Number | Site | Site | Site | Site | Sequence (5′ to 3′) | UTC) | No. |
| 1342103 | 144725 | 144742 | N/A | N/A | GCGCTCCACCCCATCCAA | 101 | 740 |
| 1342104 | 144761 | 144778 | N/A | N/A | ACAGTACCCATAATAAAG | 72 | 741 |
| 1342106 | 144721 | 144738 | N/A | N/A | TCCACCCCATCCAAGTTG | 73 | 742 |
| 1342112 | 144756 | 144773 | N/A | N/A | ACCCATAATAAAGGGCTC | 97 | 743 |
| 1342120 | 144681 | 144698 | N/A | N/A | ATAGAAATATATAGTTTG | 94 | 744 |
| 1342121 | 144701 | 144718 | N/A | N/A | GCAAGATTATCCTATACA | 77 | 745 |
| 1366951 | 152906 | 152923 | N/A | N/A | AGCATGAATTTCAGTTTA | 78 | 746 |
| 1366956 | 95421 | 95438 | N/A | N/A | CTAACATTGAAAAGCCCA | 218 | 747 |
| 1366960 | 95461 | 95478 | N/A | N/A | ACAAAAGAAAATCAAAAT | 187 | 748 |
| 1366979 | 110774 | 110791 | N/A | N/A | TTTACCTTCCAATATGCT | 33 | 749 |
| 1366986 | 144765 | 144782 | N/A | N/A | TAATACAGTACCCATAAT | 50 | 750 |
| 1366987 | 95449 | 95466 | N/A | N/A | CAAAATCCAAGTGTTATA | 73 | 751 |
| 1366989 | 110814 | 110831 | N/A | N/A | TAAACAGTTTTTCAAGCA | 71 | 752 |
| 1366993 | 152918 | 152935 | N/A | N/A | AAAGTTGGAATGAGCATG | 109 | 753 |
| 1366994 | 95441 | 95458 | N/A | N/A | AAGTGTTATATTACAAAA | 81 | 754 |
| 1366998 | 152927 | 152944 | N/A | N/A | TTGAAGAGCAAAGTTGGA | 61 | 755 |
| 1367006 | 95453 | 95470 | N/A | N/A | AAATCAAAATCCAAGTGT | 54 | 756 |
| 1367009 | 152949 | 152966 | N/A | N/A | AAATACTACATCTTACAA | 23 | 757 |
| 1367012 | 110802 | 110819 | N/A | N/A | CAAGCAGAAAATTTGAAG | 80 | 758 |
| 1367013 | 110786 | 110803 | N/A | N/A | AGACTAAACACATTTACC | 61 | 759 |
| 1367019 | 144757 | 144774 | N/A | N/A | TACCCATAATAAAGGGCT | 70 | 760 |
| 1367027 | 152910 | 152927 | N/A | N/A | AATGAGCATGAATTTCAG | 74 | 761 |
| 1367028 | 91250 | 91267 | N/A | N/A | TCCCTGTAATCCCAGTTG | 61 | 762 |
| 1367030 | 95457 | 95474 | N/A | N/A | AAGAAAATCAAAATCCAA | 76 | 763 |
| 1367041 | 110778 | 110795 | N/A | N/A | CACATTTACCTTCCAATA | 50 | 764 |
| 1367046 | 152889 | 152906 | N/A | N/A | AGGTCTTGGAGCCTGAAA | 55 | 765 |
| 1367051 | 110830 | 110847 | N/A | N/A | TATACACAATTAAATGTA | 74 | 766 |
| 1367052 | 144685 | 144702 | N/A | N/A | CAAAATAGAAATATATAG | 126 | 767 |
| 1367055 | 91219 | 91236 | N/A | N/A | GGCAGGAGAACAGGAGAA | 89 | 768 |
| 1367062 | 152940 | 152957 | N/A | N/A | ATCTTACAAAGTTTTGAA | 67 | 769 |
| 1367063 | 144689 | 144706 | N/A | N/A | TATACAAAATAGAAATAT | 78 | 770 |
| 1367064 | 110806 | 110823 | N/A | N/A | TTTTCAAGCAGAAAATTT | 65 | 771 |
| 1367065 | 95433 | 95450 | N/A | N/A | TATTACAAAAAGCTAACA | 56 | 772 |
| 1367067 | 110826 | 110843 | N/A | N/A | CACAATTAAATGTAAACA | 68 | 773 |
| 1367070 | 152914 | 152931 | N/A | N/A | TTGGAATGAGCATGAATT | 71 | 774 |
| 1367074 | 152870 | 152887 | N/A | N/A | AGGCTGAAATCAAATAAA | 70 | 775 |
| 1367077 | 152880 | 152897 | N/A | N/A | AGCCTGAAAGAGGCTGAA | 58 | 776 |
| 1367086 | 110782 | 110799 | N/A | N/A | TAAACACATTTACCTTCC | 129 | 777 |
| 1367092 | 144709 | 144726 | N/A | N/A | AAGTTGGAGCAAGATTAT | 229 | 778 |
| 1367094 | 110794 | 110811 | N/A | N/A | AAATTTGAAGACTAAACA | 54 | 779 |
| 1367095 | 152884 | 152901 | N/A | N/A | TTGGAGCCTGAAAGAGGC | 126 | 780 |
| 1367106 | 152958 | 152975 | N/A | N/A | GTTGATAGAAAATACTAC | 63 | 781 |
| 1367111 | 95477 | 95494 | N/A | N/A | TATTGTTAAGGAACACAC | 80 | 782 |
| 1367112 | 91143 | 91160 | N/A | N/A | GCCTGGGTGATGAGAGCA | 45 | 783 |
| 1367118 | 152894 | 152911 | N/A | N/A | AGTTTAGGTCTTGGAGCC | 146 | 784 |
| 1367130 | 91215 | 91232 | N/A | N/A | GGAGAACAGGAGAATCGC | 53 | 785 |
| 1367134 | 95429 | 95446 | N/A | N/A | ACAAAAAGCTAACATTGA | 357 | 786 |
| 1367142 | 95445 | 95462 | N/A | N/A | ATCCAAGTGTTATATTAC | 87 | 787 |
| 1367148 | 152962 | 152979 | N/A | N/A | GAAGGTTGATAGAAAATA | 172 | 788 |
| 1367149 | 95473 | 95490 | N/A | N/A | GTTAAGGAACACACAAAA | 48 | 789 |
| 1367154 | 152936 | 152953 | N/A | N/A | TACAAAGTTTTGAAGAGC | 37 | 790 |
| 1367156 | 152953 | 152970 | N/A | N/A | TAGAAAATACTACATCTT | 33 | 791 |
| 1367158 | 110822 | 110839 | N/A | N/A | ATTAAATGTAAACAGTTT | 54 | 792 |
| 1367159 | 95469 | 95486 | N/A | N/A | AGGAACACACAAAAGAAA | 48 | 793 |
| 1367160 | 152931 | 152948 | N/A | N/A | AGTTTTGAAGAGCAAAGT | 86 | 794 |
| 1367168 | 144693 | 144710 | N/A | N/A | ATCCTATACAAAATAGAA | 67 | 795 |
| 1367170 | 91139 | 91156 | N/A | N/A | GGGTGATGAGAGCAAAAC | 37 | 796 |
| 1367178 | 95465 | 95482 | N/A | N/A | ACACACAAAAGAAAATCA | 39 | 797 |
| 1367181 | 110810 | 110827 | N/A | N/A | CAGTTTTTCAAGCAGAAA | 72 | 798 |
| 1367183 | 152875 | 152892 | N/A | N/A | GAAAGAGGCTGAAATCAA | 49 | 799 |
| 1367186 | 144697 | 144714 | N/A | N/A | GATTATCCTATACAAAAT | 77 | 800 |
| 1367190 | 152866 | 152883 | N/A | N/A | TGAAATCAAATAAAATAG | 38 | 801 |
| 1367199 | 110818 | 110835 | N/A | N/A | AATGTAAACAGTTTTTCA | 118 | 802 |
| 1367201 | 152898 | 152915 | N/A | N/A | TTTCAGTTTAGGTCTTGG | 65 | 803 |
| 1367202 | 91135 | 91152 | N/A | N/A | GATGAGAGCAAAACTCCA | 104 | 804 |
| 1367213 | 110798 | 110815 | N/A | N/A | CAGAAAATTTGAAGACTA | 65 | 805 |
| 1367217 | 152902 | 152919 | N/A | N/A | TGAATTTCAGTTTAGGTC | 53 | 806 |
| 1367224 | 110790 | 110807 | N/A | N/A | TTGAAGACTAAACACATT | 50 | 807 |
| 1367226 | 95481 | 95498 | N/A | N/A | AGGTTATTGTTAAGGAAC | 123 | 808 |
| 1367230 | 152922 | 152939 | N/A | N/A | GAGCAAAGTTGGAATGAG | 72 | 809 |
| 1367234 | 95437 | 95454 | N/A | N/A | GTTATATTACAAAAAGCT | 80 | 810 |
| 1367235 | 95425 | 95442 | N/A | N/A | AAAGCTAACATTGAAAAG | 79 | 811 |
| 1367237 | 152945 | 152962 | N/A | N/A | ACTACATCTTACAAAGTT | 92 | 812 |
| 1367238 | 152860 | 152877 | N/A | N/A | CAAATAAAATAGGTTTAT | 105 | 813 |
| 1367239 | 144713 | 144730 | N/A | N/A | ATCCAAGTTGGAGCAAGA | 112 | 814 |
| 1367241 | 144717 | 144734 | N/A | N/A | CCCCATCCAAGTTGGAGC | 212 | 815 |
| 1367242 | 144705 | 144722 | N/A | N/A | TGGAGCAAGATTATCCTA | 147 | 816 |

TABLE 11
Effect of modified oligonucleotides on amount of human SCNIA RNA
in HepG2 cells

| | SEQ | SEQ | SEQ | SEQ | | | |
|---|---|---|---|---|---|---|---|
| | ID NO: | ID NO: | ID NO: | ID NO: | | SCNIA | SEQ |
| Compound | 1 Start | 1 Stop | 2 Start | 2 Stop | | (% | ID |
| Number | Site | Site | Site | Site | Sequence (5′ to 3′) | UTC) | No. |
| 1342083 | 144866 | 144883 | N/A | N/A | GTATTTTCCCTACTGTGG | 43 | 817 |
| 1342084 | 144871 | 144888 | N/A | N/A | ATAATGTATTTTCCCTAC | 261 | 818 |
| 1342085 | 144886 | 144903 | N/A | N/A | GGGATTAGGATGTAAATA | 105 | 819 |
| 1342086 | 144920 | 144937 | N/A | N/A | TTTTTCAAATGAAATTTA | 100 | 820 |
| 1342087 | 144861 | 144878 | N/A | N/A | TTCCCTACTGTGGTGCAA | 44 | 821 |
| 1342088 | 144876 | 144893 | N/A | N/A | TGTAAATAATGTATTTTC | 71 | 822 |
| 1342089 | 144911 | 144928 | N/A | N/A | TGAAATTTAAGACAATTG | 51 | 823 |
| 1342090 | 144881 | 144898 | N/A | N/A | TAGGATGTAAATAATGTA | 96 | 824 |
| 1342091 | 144902 | 144919 | N/A | N/A | AGACAATTGAAAAGAGGG | 91 | 825 |
| 1342092 | 144916 | 144933 | N/A | N/A | TCAAATGAAATTTAAGAC | 68 | 826 |
| 1342093 | 144906 | 144923 | N/A | N/A | TTTAAGACAATTGAAAAG | 94 | 827 |
| 1342094 | 144821 | 144838 | N/A | N/A | TCCCTCACCCCACTACAC | 80 | 828 |
| 1342095 | 144791 | 144808 | N/A | N/A | GCAAGGATTAAAGGTAGC | 83 | 829 |
| 1342096 | 144816 | 144833 | N/A | N/A | CACCCCACTACACATAAG | 58 | 830 |
| 1342097 | 144784 | 144801 | N/A | N/A | TTAAAGGTAGCAAAAGGG | 101 | 831 |
| 1342098 | 144796 | 144813 | N/A | N/A | ACAGTGCAAGGATTAAAG | 120 | 832 |
| 1342099 | 144801 | 144818 | N/A | N/A | AAGTCACAGTGCAAGGAT | 8 | 833 |
| 1342100 | 144806 | 144823 | N/A | N/A | CACATAAGTCACAGTGCA | 49 | 834 |
| 1342101 | 144786 | 144803 | N/A | N/A | GATTAAAGGTAGCAAAAG | 88 | 835 |
| 1342102 | 144811 | 144828 | N/A | N/A | CACTACACATAAGTCACA | 82 | 836 |
| 1342103 | 144725 | 144742 | N/A | N/A | GCGCTCCACCCCATCCAA | 107 | 740 |
| 1342104 | 144761 | 144778 | N/A | N/A | ACAGTACCCATAATAAAG | 55 | 741 |
| 1342105 | 144706 | 144723 | N/A | N/A | TTGGAGCAAGATTATCCT | 88 | 510 |
| 1342106 | 144721 | 144738 | N/A | N/A | TCCACCCCATCCAAGTTG | 71 | 742 |
| 1342107 | 144766 | 144783 | N/A | N/A | GTAATACAGTACCCATAA | 102 | 511 |
| 1342108 | 144716 | 144733 | N/A | N/A | CCCATCCAAGTTGGAGCA | 70 | 664 |
| 1342109 | 144753 | 144770 | N/A | N/A | CATAATAAAGGGCTCAGG | 89 | 512 |
| 1342110 | 144711 | 144728 | N/A | N/A | CCAAGTTGGAGCAAGATT | 97 | 587 |
| 1342111 | 144768 | 144785 | N/A | N/A | GGGTAATACAGTACCCAT | 51 | 665 |
| 1342112 | 144756 | 144773 | N/A | N/A | ACCCATAATAAAGGGCTC | 84 | 743 |
| 1342113 | 144666 | 144683 | N/A | N/A | TTGTTATTAGTTAGAAAT | 106 | 837 |
| 1342114 | 144661 | 144678 | N/A | N/A | ATTAGTTAGAAATCTGAT | 88 | 838 |
| 1342115 | 144691 | 144708 | N/A | N/A | CCTATACAAAATAGAAAT | 97 | 588 |
| 1342116 | 144676 | 144693 | N/A | N/A | AATATATAGTTTGTTATT | 68 | 839 |
| 1342117 | 144671 | 144688 | N/A | N/A | ATAGTTTGTTATTAGTTA | 94 | 840 |
| 1342118 | 144686 | 144703 | N/A | N/A | ACAAAATAGAAATATATA | 117 | 513 |
| 1342119 | 144656 | 144673 | N/A | N/A | TTAGAAATCTGATATGAC | 97 | 841 |
| 1342120 | 144681 | 144698 | N/A | N/A | ATAGAAATATATAGTTTG | 100 | 744 |
| 1342121 | 144701 | 144718 | N/A | N/A | GCAAGATTATCCTATACA | 51 | 745 |
| 1342122 | 144696 | 144713 | N/A | N/A | ATTATCCTATACAAAATA | 70 | 666 |
| 1342123 | 144646 | 144663 | N/A | N/A | GATATGACAGAACATTTG | 74 | 842 |
| 1342124 | 144615 | 144632 | N/A | N/A | TTCATGATGCTCTCCGTC | 67 | 843 |
| 1342125 | 144613 | 144630 | N/A | N/A | CATGATGCTCTCCGTCTG | 50 | 844 |
| 1342126 | 144617 | 144634 | N/A | N/A | TGTTCATGATGCTCTCCG | 68 | 845 |
| 1342127 | 144619 | 144636 | N/A | N/A | TTTGTTCATGATGCTCTC | 65 | 846 |
| 1342128 | 144633 | 144650 | N/A | N/A | ATTTGGTGTTACTTTTTG | 67 | 847 |
| 1342129 | 144621 | 144638 | N/A | N/A | TTTTTGTTCATGATGCTC | 34 | 848 |
| 1342130 | 144636 | 144653 | N/A | N/A | AACATTTGGTGTTACTTT | 82 | 849 |
| 1342131 | 144641 | 144658 | N/A | N/A | GACAGAACATTTGGTGTT | 57 | 850 |
| 1342132 | 144651 | 144668 | N/A | N/A | AATCTGATATGACAGAAC | 58 | 851 |
| 1342133 | 144609 | 144626 | N/A | N/A | ATGCTCTCCGTCTGTTTC | 70 | 852 |
| 1342134 | 144571 | 144588 | N/A | N/A | TTCTTTTGAAAAAGAAGG | 107 | 853 |
| 1342135 | 144591 | 144608 | N/A | N/A | CCTATCCATTAAGACTTG | 56 | 854 |
| 1342136 | 144606 | 144623 | N/A | N/A | CTCTCCGTCTGTTTCCCT | 103 | 855 |
| 1342137 | 144581 | 144598 | N/A | N/A | AAGACTTGAGTTCTTTTG | 51 | 856 |
| 1342138 | 144601 | 144618 | N/A | N/A | CGTCTGTTTCCCTATCCA | 101 | 857 |
| 1342139 | 144586 | 144603 | N/A | N/A | CCATTAAGACTTGAGTTC | 40 | 858 |
| 1342140 | 144596 | 144613 | N/A | N/A | GTTTCCCTATCCATTAAG | 68 | 859 |
| 1342141 | 144576 | 144593 | N/A | N/A | TTGAGTTCTTTTGAAAAA | 98 | 860 |
| 1342142 | 144611 | 144628 | N/A | N/A | TGATGCTCTCCGTCTGTT | 35 | 861 |
| 1342143 | 144856 | 144873 | N/A | N/A | TACTGTGGTGCAATAATA | 69 | 862 |
| 1342144 | 144536 | 144553 | N/A | N/A | GTTAGGAAATTAAAAATA | 94 | 863 |
| 1342145 | 144556 | 144573 | N/A | N/A | AGGTATTTAAGATTTCCT | 56 | 864 |
| 1342146 | 144848 | 144865 | N/A | N/A | TGCAATAATAGTACCCTT | 72 | 865 |
| 1342147 | 144546 | 144563 | N/A | N/A | GATTTCCTAGGTTAGGAA | 62 | 866 |
| 1342148 | 144561 | 144578 | N/A | N/A | AAAGAAGGTATTTAAGAT | 79 | 867 |
| 1342149 | 144851 | 144868 | N/A | N/A | TGGTGCAATAATAGTACC | 80 | 868 |
| 1342150 | 144541 | 144558 | N/A | N/A | CCTAGGTTAGGAAATTAA | 79 | 869 |
| 1342151 | 144551 | 144568 | N/A | N/A | TTTAAGATTTCCTAGGTT | 56 | 870 |
| 1342152 | 144566 | 144583 | N/A | N/A | TTGAAAAAGAAGGTATTT | 60 | 871 |
| 1342153 | 144836 | 144853 | N/A | N/A | ACCCTTCCCAATCCCTCC | 63 | 872 |
| 1342154 | 144840 | 144857 | N/A | N/A | TAGTACCCTTCCCAATCC | 75 | 873 |
| 1342155 | 144844 | 144861 | N/A | N/A | ATAATAGTACCCTTCCCA | 34 | 874 |
| 1342156 | 144846 | 144863 | N/A | N/A | CAATAATAGTACCCTTCC | 45 | 875 |
| 1342157 | 144831 | 144848 | N/A | N/A | TCCCAATCCCTCCCTCAC | 65 | 876 |
| 1342158 | 144826 | 144843 | N/A | N/A | ATCCCTCCCTCACCCCAC | 63 | 877 |
| 1342159 | 144838 | 144855 | N/A | N/A | GTACCCTTCCCAATCCCT | 43 | 878 |
| 1342160 | 144842 | 144859 | N/A | N/A | AATAGTACCCTTCCCAAT | 139 | 879 |

### Example 3: Activity of Human-Mouse Cross-Reactive Modified Oligonucleotides Against Mouse SCN1A in Primary Mouse Cerebral Neuron Cells, Single Dose, In Vitro

Certain modified oligonucleotides described above were tested for their effect on mouse SCN1A RNA levels in vitro.

The modified oligonucleotides in the tables below are 18 nucleosides in length. Each nucleoside comprises a 2′-MOE sugar moiety. The intemucleoside linkages throughout each modified oligonucleotide are phosphorothioate internucleoside linkages. All cytosine nucleobases throughout each modified oligonucleotide are 5-methylcytosines.

“Start site” indicates the 5′-most nucleoside of the target sequence to which the modified oligonucleotide is complementary. “Stop site” indicates the 3′-most nucleoside of the target sequence to which the modified oligonucleotide is complementary. As shown in the tables below, the modified oligonucleotides are complementary to the human SCN1A genomic sequence, designated herein as SEQ ID NO: 1 (the complement of GENBANK Accession No. NC_000002.12 truncated from nucleotides 165982001 to 166152000) and the mouse SCN1A genomic sequence, designated herein as SEQ ID NO: 3 (the complement of GENBANK Accession No. NC_000068.7 truncated from nucleotides 66268001 to 66444000).

Cultured primary mouse cerebral neuron cells at a density of 60,000 cells per well were treated by free uptake with 8000 nM of modified oligonucleotide. After a treatment period of approximately 3 days, RNA was isolated from the cells and the amount of SCN1A RNA that excludes the mouse form of NIE-1 (NIE-1⁻), and the amount of SCN1A RNA that includes the mouse form of NIE-1 (NIE-1⁺) were measured by quantitative real-time RTPCR. Mouse primer probe set RTS48951 (forward sequence CCCTAAGAGCCTTATCACGATTT, designated herein as SEQ ID NO: 24; reverse sequence GGCAAACCAGAAGCACATTC, designated herein as SEQ ID NO: 25; probe sequence AGGGTGGTTGTGAATGCCCTGTTA, designated herein as SEQ ID NO: 26) was used to measure the amount of SCN1A transcript that excludes the mouse form of NIE-1 (NIE-1⁻). Mouse primer probe set RTS48949 (forward sequence AGCCCTTTATTATGGGTGGTT, designated herein as SEQ ID NO: 21; reverse sequence CCAGAATATAAGGCAAACCAGAAG, designated herein as SEQ ID NO: 22; probe sequence TGGATGGAATTGCTCCTAACAGGGC, designated herein as SEQ ID NO: 23) was used to measure the amount of SCN1A transcript that includes the mouse form of NIE-1 (NIE-1⁺). SCN1A RNA levels were normalized to total RNA content, as measured by RIBOGREEN@. SCN1A NIE-1⁻RNA and NIE-1⁺RNA are presented as % of the average of untreated control (% UTC). Values marked with a “ [symbol] ” result from oligonucleotides that are complementary to the amplicon region of the primer probe set. Additional assays may be used to measure the potency and efficacy of the modified oligonucleotides complementary to the amplicon region.

As shown in the tables below, certain modified oligonucleotides complementary to a SCN1A nucleic acid increased the amount ofSCN1A RNA that excludes the mouse form of NIE-1 (NIE-1⁻) and reduced the amount ofSCN1A RNA that includes the mouse form of NIE-1 (NIE-1⁺), compared to untreated control.

TABLE 12
Effect of modified oligonucleotides on the amount of mouse SCN1A RNA excluding
NIE-1 (NIE-1⁻) and the amount of mouse SCNIA RNA including NIE-1 (NIE-1⁻)
in primary mouse cerebral neuron cells

| | SEQ | SEQ | SEQ | SEQ | | | | |
|---|---|---|---|---|---|---|---|---|
| | ID NO: | ID NO: | ID NO: | ID NO: | | SCNIA | SCNIA | |
| | 1 Start | 1 Stop | 3 Start | 3 Stop | | (% UTC) | (% UTC) | SEQ |
| Compound | Site | Site | Site | Site | | RTS48949 | RTS48951 | ID |
| Number | Human | Human | Mouse | Mouse | Sequence (5′ to 3′) | NIE-1⁺ | NIE-1⁻ | No. |
| 1342097 | 144784 | 144801 | 150182 | 150199 | TTAAAGGTAGCAAAAGGG | 96 | 69 | 831 |
| 1342104 | 144761 | 144778 | 150159 | 150176 | ACAGTACCCATAATAAAG | 68‡ | 248 | 741 |
| 1342105 | 144706 | 144723 | 150104 | 150121 | TTGGAGCAAGATTATCCT | 30 | 549 | 510 |
| 1342106 | 144721 | 144738 | 150119 | 150136 | TCCACCCCATCCAAGTTG | 111 | 155 | 742 |
| 1342107 | 144766 | 144783 | 150164 | 150181 | GTAATACAGTACCCATAA | 551 | 346 | 511 |
| 1342108 | 144716 | 144733 | 150114 | 150131 | CCCATCCAAGTTGGAGCA | 46 | 349 | 664 |
| 1342110 | 144711 | 144728 | 150109 | 150126 | CCAAGTTGGAGCAAGATT | 15 | 647 | 587 |
| 1342111 | 144768 | 144785 | 150166 | 150183 | GGGTAATACAGTACCCAT | 104‡ | 107 | 665 |
| 1366954 | 144703 | 144720 | 150101 | 150118 | GAGCAAGATTATCCTATA | 16 | 644 | 590 |
| 1366958 | 144722 | 144739 | 150120 | 150137 | CTCCACCCCATCCAAGTT | 113 | 122 | 515 |
| 1366971 | 144762 | 144779 | 150160 | 150177 | TACAGTACCCATAATAAA | 72‡ | 465 | 519 |
| 1366982 | 144710 | 144727 | 150108 | 150125 | CAAGTTGGAGCAAGATTA | 9 | 696 | 523 |
| 1366986 | 144765 | 144782 | 150163 | 150180 | TAATACAGTACCCATAAT | 52‡ | 333 | 750 |
| 1367004 | 144682 | 144699 | 150080 | 150097 | AATAGAAATATATAGTTT | 60 | 264 | 529 |
| 1367019 | 144757 | 144774 | 150155 | 150172 | TACCCATAATAAAGGGCT | 54‡ | 125 | 760 |
| 1367043 | 144712 | 144729 | 150110 | 150127 | TCCAAGTTGGAGCAAGAT | 54 | 450 | 692 |
| 1367047 | 144758 | 144775 | 150156 | 150173 | GTACCCATAATAAAGGGC | 86‡ | 146 | 539 |
| 1367071 | 144760 | 144777 | 150158 | 150175 | CAGTACCCATAATAAAGG | 67‡ | 224 | 694 |
| 1367076 | 144704 | 144721 | 150102 | 150119 | GGAGCAAGATTATCCTAT | 29 | 510 | 696 |
| 1367089 | 144684 | 144701 | 150082 | 150099 | AAAATAGAAATATATAGT | 83 | 180 | 703 |
| 1367092 | 144709 | 144726 | 150107 | 150124 | AAGTTGGAGCAAGATTAT | 14 | 642 | 778 |
| 1367098 | 144714 | 144731 | 150112 | 150129 | CATCCAAGTTGGAGCAAG | 41 | 421 | 550 |
| 1367121 | 144719 | 144736 | 150117 | 150134 | CACCCCATCCAAGTTGGA | 110 | 215 | 628 |
| 1367131 | 144724 | 144741 | 150122 | 150139 | CGCTCCACCCCATCCAAG | 82 | 86 | 716 |
| 1367135 | 144759 | 144776 | 150157 | 150174 | AGTACCCATAATAAAGGG | 67‡ | 214 | 634 |
| 1367144 | 144720 | 144737 | 150118 | 150135 | CCACCCCATCCAAGTTGG | 81 | 182 | 722 |
| 1367155 | 144723 | 144740 | 150121 | 150138 | GCTCCACCCCATCCAAGT | 128 | 102 | 637 |
| 1367174 | 144763 | 144780 | 150161 | 150178 | ATACAGTACCCATAATAA | 57‡ | 395 | 642 |
| 1367182 | 144715 | 144732 | 150113 | 150130 | CCATCCAAGTTGGAGCAA | 44 | 431 | 645 |
| 1367184 | 144767 | 144784 | 150165 | 150182 | GGTAATACAGTACCCATA | 851 | 210 | 646 |
| 1367189 | 144718 | 144735 | 150116 | 150133 | ACCCCATCCAAGTTGGAG | 117 | 185 | 575 |
| 1367218 | 144707 | 144724 | 150105 | 150122 | GTTGGAGCAAGATTATCC | 9 | 727‡ | 655 |
| 1367219 | 144764 | 144781 | 150162 | 150179 | AATACAGTACCCATAATA | 97‡ | 511 | 736 |
| 1367221 | 144683 | 144700 | 150081 | 150098 | AAATAGAAATATATAGTT | 74 | 294 | 656 |
| 1367239 | 144713 | 144730 | 150111 | 150128 | ATCCAAGTTGGAGCAAGA | 37 | 514 | 814 |
| 1367241 | 144717 | 144734 | 150115 | 150132 | CCCCATCCAAGTTGGAGC | 99 | 184 | 815 |
| 1367242 | 144705 | 144722 | 150103 | 150120 | TGGAGCAAGATTATCCTA | 72 | 430 | 816 |
| 1369834 | 144772 | 144789 | 150170 | 150187 | AAAGGGGTAATACAGTAC | 47 | 324 | 880 |
| 1369835 | 144779 | 144796 | 150177 | 150194 | GGTAGCAAAAGGGGTAAT | 24 | 463 | 881 |
| 1369836 | 144774 | 144791 | 150172 | 150189 | CAAAAGGGGTAATACAGT | 44 | 448 | 882 |
| 1369838 | 144771 | 144788 | 150169 | 150186 | AAGGGGTAATACAGTACC | 691 | 283 | 883 |
| 1369840 | 144781 | 144798 | 150179 | 150196 | AAGGTAGCAAAAGGGGTA | 102 | 64 | 884 |
| 1369841 | 144776 | 144793 | 150174 | 150191 | AGCAAAAGGGGTAATACA | 19 | 579 | 885 |
| 1369851 | 144770 | 144787 | 150168 | 150185 | AGGGGTAATACAGTACCC | 90‡ | 148 | 886 |
| 1369854 | 144769 | 144786 | 150167 | 150184 | GGGGTAATACAGTACCCA | 97‡ | 137 | 887 |
| 1369856 | 144775 | 144792 | 150173 | 150190 | GCAAAAGGGGTAATACAG | 48 | 317 | 888 |
| 1369870 | 144782 | 144799 | 150180 | 150197 | AAAGGTAGCAAAAGGGGT | 63 | 53 | 889 |

### Example 4: Activity of Modified Oligonucleotides Targeting Human SCN1A in HepG2 Cells, Multiple Dose, In Vitro

Certain modified oligonucleotides described in the studies above were selected and tested at various doses in HepG2 cells.

The modified oligonucleotides were tested in a series of experiments using the same culture conditions. The results for each experiment are presented in separate tables shown below. Cultured HepG2 cells at a density of 20,000 cells per well were transfected using electroporation with modified oligonucleotides diluted to different concentrations as specified in the tables below. After a treatment period of approximately 24 hours, full-length SCN1A RNA levels were measured as previously described using the human RTS48189 primer-probe set. Full-length SCN1A RNA levels were normalized to total RNA, as measured by RIBOGREEN®. Full-length SCN1A RNA is presented as % of the average of untreated control (%% UTC). Also provided is the fold increase of full-length SCN1A RNA over the untreated control (fold increase over UTC) at the 20 μM dose. As shown in the tables below, certain modified oligonucleotides complementary to SCN1A RNA increased the amount of human full-length SCN1A RNA, compared to untreated control.

TABLE 13
Effect of modified oligonucleotides on the amount
of human full-length SCN1A RNA in HepG2 cells,
multiple dose

| | | | | | Fold |
|---|---|---|---|---|---|
| | | | | | increase |
| | % UTC | | | | over |
| | 312.5 | 1250 | 5000 | 20000 | UTC @ |
| Compound No. | nM | nM | nM | nM | 20 μM |
| 1342118 | 78 | 88 | 96 | 73 | 0.7 |
| 1367236 | 148 | 91 | 105 | 147 | 1.5 |
| 1367011 | 100 | 85 | 85 | 89 | 0.9 |
| 1366977 | 81 | 64 | 19 | 41 | 0.4 |
| 1367098 | 138 | 80 | 96 | 117 | 1.2 |
| 1367121 | 66 | 79 | 95 | 64 | 0.6 |
| 1367163 | 81 | 138 | 61 | 115 | 1.2 |
| 1367214 | 104 | 81 | 69 | 48 | 0.5 |
| 1366982 | 116 | 128 | 142 | 137 | 1.4 |
| 1367155 | 89 | 67 | 144 | 126 | 1.3 |
| 1367182 | 114 | 80 | 64 | 38 | 0.4 |
| 1366952 | 86 | 88 | 66 | 114 | 1.1 |
| 1367014 | 80 | 63 | 85 | 49 | 0.5 |
| 1367184 | 104 | 71 | 67 | 45 | 0.4 |
| 1367240 | 86 | 90 | 95 | 124 | 1.2 |
| 1366980 | 78 | 55 | 79 | 73 | 0.7 |
| 1367134 | 124 | 58 | 78 | 73 | 0.7 |
| 1367147 | 140 | 110 | 123 | 207 | 2.1 |
| 1367232 | 121 | 84 | 116 | 72 | 0.7 |

TABLE 14
Effect of modified oligonucleotides on the amount
of human full-length SCN1A RNA in HepG2 cells,
multiple dose

| | | | | | Fold |
|---|---|---|---|---|---|
| | | | | | increase |
| | % UTC | | | | over |
| | 312.5 | 1250 | 5000 | 20000 | UTC @ |
| Compond No. | nM | nM | nM | nM | 20 μM |
| 1367020 | 63 | 108 | 64 | 92 | 0.9 |
| 1366961 | 98 | 76 | 64 | 62 | 0.6 |
| 1367059 | 73 | 138 | 110 | 169 | 1.7 |
| 1367040 | 56 | 41 | 53 | 76 | 0.8 |
| 1367135 | 110 | 130 | 120 | 136 | 1.4 |
| 1366962 | 65 | 99 | 79 | 147 | 1.5 |
| 1367107 | 93 | 103 | 93 | 153 | 1.5 |
| 1367119 | 101 | 99 | 89 | 86 | 0.9 |
| 1367131 | 84 | 167 | 106 | 178 | 1.8 |
| 1367032 | 56 | 55 | 103 | 67 | 0.7 |
| 1366960 | 89 | 105 | 74 | 38 | 0.4 |
| 1367211 | 100 | 113 | 118 | 131 | 1.3 |
| 1367092 | 87 | 83 | 179 | 95 | 0.9 |
| 1367148 | 86 | 116 | 81 | 44 | 0.4 |
| 1367132 | 107 | 117 | 75 | 142 | 1.4 |
| 1366956 | 80 | 87 | 58 | 63 | 0.6 |
| 1367134 | 89 | 106 | 89 | 83 | 0.8 |
| 1367241 | 104 | 110 | 73 | 86 | 0.9 |

TABLE 15
Effect of modified oligonucleotides on the amount
of human full-length SCN1A RNA expression in HepG2
cells, multiple dose

| | | | | | Fold |
|---|---|---|---|---|---|
| | | | | | increase |
| | % UTC | | | | over |
| | 312.5 | 1250 | 5000 | 20000 | UTC @ |
| Compound No. | nM | nM | nM | nM | 20 μM |
| 1367242 | 197 | 189 | 403 | 388 | 3.9 |
| 1366993 | 59 | 78 | 77 | 63 | 0.6 |
| 1367118 | 63 | 79 | 65 | 80 | 0.8 |
| 1367238 | 77 | 81 | 84 | 65 | 0.7 |
| 1367086 | 95 | 65 | 69 | 111 | 1.1 |
| 1366994 | 100 | 65 | 69 | 52 | 0.5 |
| 1367095 | 113 | 155 | 153 | 170 | 1.7 |
| 1342084 | 133 | 100 | 132 | 207 | 2.1 |
| 1367052 | 108 | 113 | 118 | 114 | 1.1 |
| 1342160 | 103 | 119 | 112 | 66 | 0.7 |
| 1342120 | 85 | 79 | 78 | 103 | 1.0 |
| 1367226 | 70 | 82 | 125 | 66 | 0.7 |
| 1342098 | 92 | 63 | 95 | 78 | 0.8 |
| 1342141 | 46 | 76 | 61 | 49 | 0.5 |
| 1367199 | 81 | 109 | 133 | 136 | 1.4 |
| 1342118 | 120 | 99 | 93 | 95 | 0.9 |
| 1367134 | 70 | 61 | 62 | 124 | 1.2 |
| 1367239 | 122 | 109 | 105 | 180 | 1.8 |
| 1342103 | 115 | 110 | 94 | 90 | 0.9 |

### Example 5: Activity of Human-Mouse Cross-Reactive Modified Oligonucleotides Against Mouse SCN1A in Primary Mouse Cerebral Neuron Cells, Multiple Dose, In Vitro

Certain modified oligonucleotides described in the studies above were tested at various doses in primary mouse cerebral neuron cells. The modified oligonucleotides were tested in a series of experiments using the same culture conditions.

Cultured primary mouse cerebral neuron cells at a density of 60,000 cells per well were treated by free uptake with modified oligonucleotides diluted to different concentrations as specified in the tables below. After a treatment period of approximately 3 days, mouse primer probe set RTS48951 (described herein above) was used to measure the amount of mouse SCN1A RNA that excludes NIE-1 (NIE-1⁻), and mouse primer probe set RTS48949 (described herein above) was used to measure the amount of mouse SCN1A RNA that includes NIE-1 (NIE-1⁺). SCN1A RNA levels were normalized to total RNA content, as measured by RIBOGREEN®. SCN1A RNA is presented as % of the average of untreated control (% UTC). Values marked with a “ [symbol] ” result from oligonucleotides that are complementary to the amplicon region of the primer probe set. Additional assays may be used to measure the potency and efficacy of the modified oligonucleotides complementary to the amplicon region. Also provided is the fold increase of (NIE-1⁻)SCN1A RNA over the untreated control (fold increase over UTC) at the 8 μM dose. The half maximal inhibitory concentration (IC₅₀) was calculated using a linear regression on a log/linear plot of the data in Excel; an IC₅₀ of >8 μM in the tables below indicates that the value was not calculatable.

As shown in the tables below, certain modified oligonucleotides complementary to a SCN1A nucleic acid increased the amount of SCN1A RNA that excludes the mouse form of NIE-1 (NIE-1⁻) and reduced the amount of SCN1A RNA that includes the mouse form of NIE-1 (NIE-1⁺), compared to untreated control.

TABLE 16
Effect of modified oligonucleotides on the amount of mouse SCN1A
RNA excluding NIE-1 (NIE-1⁻) and the amount of SCN1A RNA including
(NIE-1⁺) in primary mouse cerebral neuron cells, multiple doses

| | | | | | | NIE-1⁻ | | | | |
|---|---|---|---|---|---|---|---|---|---|---|
| | NIE-1⁺ | | | | | | | | | Fold |
| | % UTC | | | | | % UTC | | | | increase |
| Compound | 64 | 320 | 1600 | 8000 | IC₅₀ | 64 | 320 | 1600 | 8000 | over UTC |
| No. | nM | nM | nM | nM | μM | nM | nM | nM | nM | 8 μM |
| 1369834 | 149 | 143 | 126 | 80 | >8.0 | 137 | 162 | 292 | 817 | 8.2 |
| 1367174 | 111‡ | 102‡ | 87‡ | 62‡ | >8.0 | 145 | 208 | 344 | 1059 | 10.6 |
| 1369856 | 133 | 142 | 100 | 55 | >8.0 | 149 | 171 | 322 | 968 | 9.7 |
| 1366971 | 100‡ | 102‡ | 92‡ | 76‡ | >8.0 | 124 | 170 | 305 | 849 | 8.5 |
| 1366986 | 89‡ | 77‡ | 75‡ | 56‡ | >8.0 | 98 | 121 | 250 | 584 | 5.8 |
| 1342107 | 79‡ | 76‡ | 74‡ | 44‡ | >8.0 | 122 | 152 | 295 | 805 | 8.0 |
| 1367242 | 97 | 80 | 70 | 39 | 4.7 | 119 | 169 | 397 | 885 | 8.8 |
| 1367219 | 79‡ | 73‡ | 71‡ | 51‡ | >8.0 | 139 | 178 | 353 | 692 | 6.9 |
| 1367004 | 81 | 86 | 81 | 75 | >8.0 | 94 | 107 | 181 | 487 | 4.9 |
| 1369838 | 149‡ | 130‡ | 130‡ | 80‡ | >8.0 | 127 | 153 | 254 | 409 | 4.1 |
| 1367221 | 96 | 103 | 111 | 74 | >8.0 | 94 | 113 | 168 | 444 | 4.4 |
| 1342104 | 103‡ | 98‡ | 97‡ | 76‡ | >8.0 | 125 | 126 | 172 | 378 | 3.8 |
| 1367071 | 123‡ | 126‡ | 120‡ | 81‡ | >8.0 | 125 | 119 | 174 | 340 | 3.4 |
| 1367135 | 140‡ | 115‡ | 88‡ | 93‡ | >8.0 | 94 | 117 | 160 | 321 | 3.2 |

TABLE 17
Effect of modified oligonucleotides on amount of mouse
SCN1A RNA including (NIE-1⁺) or excluding NIE-1
(NIE-1⁻) in primary mouse cerebral neuron cells, multiple doses

| | | | | | | NIE-1⁻ | | | | |
|---|---|---|---|---|---|---|---|---|---|---|
| | | | | | | | | | | Fold |
| | | | | | | | | | | increase |
| | NIE-1⁺ | | | | | | | | | over |
| | % UTC | | | | | % UTC | | | | UTC |
| Compound | 64 | 320 | 1600 | 8000 | IC₅₀ | 64 | 320 | 1600 | 8000 | 8 |
| No. | nM | nM | nM | nM | μM | nM | nM | nM | nM | μM |
| 1367218 | 56 | 39 | 22 | 3 | 0.1 | 252 | 346 | 634 | 1084 | 10.8 |
| 1366982 | 59 | 41 | 14 | 3 | 0.1 | 241 | 336 | 737 | 1089 | 10.9 |
| 1367092 | 60 | 46 | 20 | 2 | 0.2 | 225 | 280 | 619 | 1044 | 10.4 |
| 1342110 | 62 | 42 | 16 | 6 | 0.2 | 151 | 246 | 366 | 797 | 8.0 |
| 1366954 | 51 | 36 | 14 | 4 | 0.1 | 170 | 248 | 517 | 770 | 7.7 |
| 1369841 | 145 | 110 | 56 | 10 | 2.1 | 180 | 233 | 622 | 801 | 8.0 |
| 1369835 | 129 | 62 | 43 | 15 | 1.2 | 164 | 197 | 447 | 578 | 5.8 |
| 1342105 | 81 | 62 | 46 | 13 | 0.8 | 143 | 187 | 346 | 683 | 6.8 |
| 1367076 | 88 | 72 | 65 | 15 | 1.5 | 138 | 184 | 419 | 721 | 7.2 |
| 1367239 | 86 | 84 | 63 | 18 | 1.8 | 100 | 185 | 427 | 565 | 5.7 |
| 1367098 | 92 | 109 | 69 | 31 | 4.3 | 80 | 111 | 239 | 477 | 4.8 |
| 1369836 | 120 | 97 | 65 | 14 | 2.2 | 101 | 95 | 223 | 426 | 4.3 |
| 1367182 | 132 | 103 | 81 | 38 | 5.4 | 103 | 130 | 254 | 415 | 4.1 |
| 1367043 | 108 | 83 | 93 | 39 | >8.0 | 129 | 187 | 352 | 559 | 5.6 |
| 1342108 | 136 | 88 | 85 | 45 | 6.9 | 99 | 95 | 188 | 351 | 3.5 |

### Example 6: Activity of Modified Oligonucleotides Targeting SCN1A in Wildtype Mice

Wildtype C571BL/6 mice were divided into groups of 4 mice each. Each mouse received a single ICV bolus of Compound No. 1429226 or comparator compound 1367010 at the dose indicated in the tables below. A group of 4 mice received PBS as a negative control.

Compound No. 1429226, described hereinabove, is a modified oligonucleotide having a nucleobase sequence of (from 5′ to 3′) AGTTGGAGCAAGATTATC (SLQ ID NO: 41), wherein each nucleoside comprises a 2′-NMA sugar moiety, each intemucleoside linkage is a phosphorothioate intemucleoside linkage, and each cytosine is a 5-methyl cytosine. Comparator compound 1367010, previously described in WO 2019/040923 (incorporated herein by reference) as Compound Ex 20X+1 has a nucleobase sequence of (from 5′ to 3′) AGTTGGAGCAAGATTATC (SEQ ID NO: 41), wherein each nucleoside comprises a 2′-MOE sugar moiety and each intemucleoside linkage is a phosphorothioate intemucleoside linkage. SEQ ID NO:41 is 100% complementary to SEQ ID NO: 1, from Start Site 144708 to Stop Site 144725, and is 100% complementary to SEQ ID NO: 3 from Start Site 150106 to Stop Site 150123. “Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence.

Three weeks post treatment mice were sacrificed and RNA was extracted from cortical brain tissue and spinal cord for real-time qPCR analysis of SCN1A RNA using primer probe set RTS48951 (described herein above) to measure the amount of SCN1A RNA that excludes the mouse form of NIE-1 (NIE-1⁻) and primer probe set RTS48949 (described herein above) to measure the amount of SCN1A RNA that includes the mouse form of NIE-1 (NIE-1⁺). SCN1A RNA is presented as % of the average of untreated control (% UTC), normalized to mouse GAPDH. Mouse GAPDH was amplified using primer probe set mGapdh_LTS00102 (forward sequence GGCAAATTCAACGGCACAGT, designated herein as SEQ ID NO: 36; reverse sequence GGGTCTCGCTCCTGGAAGAT, designated herein as SEQ ID NO: 37; probe sequence AAGGCCGAGAATGGGAAGCTTGTCATC, designated herein as SEQ ID NO: 38). Each of Tables 18 and 19 represents a different experiment.

As shown in the tables below, Compound No. 1429226 is more potent than comparator compound 1367010 in this assay.

TABLE 18
Effect of modified oligonucleotides on the amount of mouse
SCN1A excluding NIE-1 (NIE-1⁻) and the amount of mouse SCN1A
RNA including (NIE-1⁺) in wildtype mice, multiple doses

| | | SPINAL CORD | | | CORTEX | | |
|---|---|---|---|---|---|---|---|
| | | | NIE-1⁺ | | | NIE-1⁺ | |
| Compound | Dose | NIE-1⁻ | | ED₅₀ | NIE-1⁻ | | ED₅₀ |
| No. | (μg) | % UTC | % UTC | (μg) | % UTC | % UTC | (μg) |
| 1367010 | 1 | 82 | 88 | 50 | 111 | 94 | 78 |
| | 3 | 93 | 91 | | 109 | 96 | |
| | 10 | 98 | 76 | | 103 | 95 | |
| | 30 | 90 | 64 | | 119 | 84 | |
| | 100 | 103 | 33 | | 149 | 38 | |
| | 300 | 104 | 23 | | 176 | 13 | |
| | 700 | 108 | 18 | | 173 | 3 | |

TABLE 19
Effect of modified oligonucleotides on amount of mouse SCN1A
excluding NIE-1 (NIE-1⁻) and the amount of mouse
SCN1A RNA including (NIE-1⁺) in wildtype mice, multiple doses

| | | SPINAL CORD | | | CORTEX | | |
|---|---|---|---|---|---|---|---|
| | | | NIE-1⁺ | | | NIE-1⁺ | |
| Compound | Dose | NIE-1⁻ | | ED₅₀ | NIE-1⁻ | | ED₅₀ |
| No. | (μg) | % UTC | % UTC | (μg) | % UTC | % UTC | (μg) |
| 1429226 | 1 | 100 | 106 | 14 | 100 | 96 | 21 |
| | 3 | 109 | 106 | | 123 | 75 | |
| | 10 | 95 | 38 | | 109 | 69 | |
| | 30 | 106 | 38 | | 162 | 43 | |
| | 100 | 105 | 17 | | 164 | 11 | |
| | 300 | 104 | 7 | | 162 | 2 | |
| | 700 | 117 | 7 | | 172 | 1 | |

### Example 7: Tolerability of Modified Oligonucleotides Complementary to SCN1A in Wild-Type Mice

Compound No. 1429226 and comparator compound 1367010, both described hereinabove, were tested in wild-type female C57/B16 mice to assess the tolerability of the oligonucleotides. Wild-type female C57/B16 mice each received a single ICV dose of either 500 μg or 700 μg of modified oligonucleotide, indicated as Dose (μg) in the tables below. Each treatment group consisted of 4 mice. A group of 4 mice received PBS as a negative control for each experiment (identified in separate tables below). At 3 hours post-injection, mice were evaluated according to seven different criteria. The criteria are (1) the mouse was bright, alert, and responsive; (2) the mouse was standing or hunched without stimuli; (3) the mouse showed any movement without stimuli; (4) the mouse demonstrated forward movement after it was lifted; (5) the mouse demonstrated any movement after it was lifted; (6) the mouse responded to tail pinching; (7) regular breathing. For each of the 7 criteria, a mouse was given a subscore of 0 if it met the criteria and 1 if it did not (the functional observational battery score or FOB). After all 7 criteria were evaluated, the scores were summed for each mouse and averaged within each treatment group. Each of Tables 20-22 represents a different experiment.

As shown in the tables below, Compound No. 1429226 is more tolerable than comparator compound 1367010 in this assay.

TABLE 20
Tolerability scores in mice

| Compound | Dose | FOB 3 |
|---|---|---|
| No. | (μg) | hour |
| PBS | N/A | 0 |
| 1367010 | 500 | 5.75 |

TABLE 21
Tolerability scores in mice

| Compound | Dose | FOB 3 |
|---|---|---|
| No. | (μg) | hour |
| PBS | N/A | 0 |
| 1367010 | 700 | 7 |

TABLE 22
Tolerability scores in mice

| Compound | Dose | FOB 3 |
|---|---|---|
| No. | (μg) | hour |
| PBS | N/A | 0 |
| 1429226 | 500 | 4 |
| 1429226 | 700 | 4 |

### Example 8: Design of Modified Oligonucleotides Complementary to a Human SCN1A Nucleic Acid

Modified oligonucleotides complementary to a human SCN1A nucleic acid are designed and synthesized as indicated in Table 23 below.

The modified oligonucleotides in Table 23 are 18, 19, or 20 nucleosides in length, as specified. The modified oligonucleotides comprise 2′-MOE sugar moieties, as specified. The sugar motif for each modified oligonucleotide is provided in the Sugar Motif column, wherein each ‘e’ represents a 2′-MOE sugar moiety. The intemucleoside linkage motif for each modified oligonucleotide is provided in the Internucleoside Linkage Motif column, wherein each ‘s’ represents a phosphorothioate intemucleoside linkage, and each ‘o’ represents a phosphodiester intemucleoside linkage. Each cytosine is a 5-methyl cytosine.

Each modified oligonucleotide listed in Table 23 below is 100% complementary to SEQ ID NO: 1 (the complement of GENBANK Accession No. NC_000002.12 truncated from nucleotides 165982001 to 166152000), and to SEQ ID NO: 3 (the complement of GENBANK Accession No. NC_000068.7 truncated from nucleotides 66268001 to 66444000) unless specifically stated otherwise. SEQ ID NO:890 is 100% complementary to SEQ ID NO: 3 from Start Site 150105 to Stop Site 150124. SEQ ID NO:891 is 100% complementary to SEQ ID NO: 3 from Start Site 150105 to Stop Site 150123. SEQ ID NO:892 is 100% complementary to SEQ ID NO: 3 from Start Site 150106 to Stop Site 150124. “Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence.

TABLE 23
2′-MOE modified oligonucleotides with mixed PS/PO internucleoside linkages

| | | | | SEQ | SEQ | |
|---|---|---|---|---|---|---|
| | Nucleobase | | Internucleoside | ID No: | ID No: | SEQ |
| Compound | Sequence | Sugar Motif | Linkage Motif | 1 Start | 1 Stop | ID |
| Number | (5′ to 3′) | (5′ to 3′) | (5′ to 3′) | Site | Site | No. |
| 1472459 | AGTTGGAGCAAGATTATC | eeeeeeeeeeeeeeeeee | soossssssssssssss | 144708 | 144725 | 41 |
| 1472453 | AGTTGGAGCAAGATTATC | eeeeeeeeeeeeeeeeee | sososssssssssssss | 144708 | 144725 | 41 |
| 1472454 | AGTTGGAGCAAGATTATC | eeeeeeeeeeeeeeeeee | sosssosssssssssss | 144708 | 144725 | 41 |
| 1472455 | AGTTGGAGCAAGATTATC | eeeeeeeeeeeeeeeeee | sosssssosssssssss | 144708 | 144725 | 41 |
| 1472460 | AGTTGGAGCAAGATTATC | eeeeeeeeeeeeeeeeee | sssoossssssssssss | 144708 | 144725 | 41 |
| 1472462 | AGTTGGAGCAAGATTATC | eeeeeeeeeeeeeeeeee | sssssssoossssssss | 144708 | 144725 | 41 |
| 1472463 | AGTTGGAGCAAGATTATC | eeeeeeeeeeeeeeeeee | sssssssssoossssss | 144708 | 144725 | 41 |
| 1472464 | AGTTGGAGCAAGATTATC | eeeeeeeeeeeeeeeeee | sssssssssssoossss | 144708 | 144725 | 41 |
| 1472456 | AGTTGGAGCAAGATTATC | eeeeeeeeeeeeeeeeee | sosssssssosssssss | 144708 | 144725 | 41 |
| 1472457 | AGTTGGAGCAAGATTATC | eeeeeeeeeeeeeeeeee | sosssssssssosssss | 144708 | 144725 | 41 |
| 1472458 | AGTTGGAGCAAGATTATC | eeeeeeeeeeeeeeeeee | sossssssssssssoss | 144708 | 144725 | 41 |
| 1472466 | AGTTGGAGCAAGATTATC | eeeeeeeeeeeeeeeeee | ssosssssssssssoss | 144708 | 144725 | 41 |
| 1472467 | AGTTGGAGCAAGATTATC | eeeeeeeeeeeeeeeeee | ssssosssssssssoss | 144708 | 144725 | 41 |
| 1472461 | AGTTGGAGCAAGATTATC | eeeeeeeeeeeeeeeeee | sssssoossssssssss | 144708 | 144725 | 41 |
| 1472468 | AGTTGGAGCAAGATTATC | eeeeeeeeeeeeeeeeee | ssssssosssssssoss | 144708 | 144725 | 41 |
| 1472472 | AGTTGGAGCAAGATTATC | eeeeeeeeeeeeeeeeee | ssssssssossssosss | 144708 | 144725 | 41 |
| 1472469 | AGTTGGAGCAAGATTATC | eeeeeeeeeeeeeeeeee | ssssssssosssssoss | 144708 | 144725 | 41 |
| 1472470 | AGTTGGAGCAAGATTATC | eeeeeeeeeeeeeeeeee | ssssssssssosssoss | 144708 | 144725 | 41 |
| 1472473 | AGTTGGAGCAAGATTATC | eeeeeeeeeeeeeeeeee | sssssssssssososss | 144708 | 144725 | 41 |
| 1472471 | AGTTGGAGCAAGATTATC | eeeeeeeeeeeeeeeeee | ssssssssssssososs | 144708 | 144725 | 41 |
| 1472465 | AGTTGGAGCAAGATTATC | eeeeeeeeeeeeeeeeee | sssssssssssssooss | 144708 | 144725 | 41 |
| 1472452 | AAGTTGGAGCAAGATTAT | eeeeeeeeeeeeeeeeeeee | ossssssssssssssssso | 144707 | 144726 | 890 |
| | CC | | | | | |
| 1472451 | AGTTGGAGCAAGATTATC | eeeeeeeeeeeeeeeeeee | ssssssssssssssssso | 144707 | 144725 | 891 |
| | C | | | | | |
| 1472450 | AAGTTGGAGCAAGATTAT | eeeeeeeeeeeeeeeeeee | osssssssssssssssss | 144708 | 144726 | 892 |
| | C | | | | | |

### Example 9: Activity and Tolerability of Modified Oligonucleotides Complementary to Human SCN1A in Wild-Type Mice

### Treatment

Modified oligonucleotides described in Table 23 were tested in wild-type female C57/B16 mice to assess the tolerability and activity of the oligonucleotides. Wild-type female C57/B16 mice each received a single ICV dose of 700 μg of modified oligonucleotide as listed in the table below. Each treatment group consisted of 3 mice. A group of 4 mice received PBS as a negative control.

### Activity

Eight weeks post treatment mice were sacrificed and RNA was extracted from cortical brain tissue for quantitative real-time RTPCR analysis of SCN1A RNA using primer probe set RTS48951 (described herein above) to measure the amount of SCN1A RNA that excludes the mouse form of NIE-1 (NIE-1⁻) and primer probe set RTS48949 (described herein above) to measure the amount of SCN1A RNA that includes the mouse form of NIE-1 (NIE-1⁺). SCN1A RNA is presented as % of the average of the PBS control (% control), normalized to mouse GAPDH. Mouse GAPDH was amplified using primer probe set mGapdh_LTS00102 (described herein above). As shown in Table 24 below, the compounds demonstrate activity in this assay.

TABLE 24
Effect of modified oligonucleotides on amount of mouse SCN1A excluding
NIE-1 (NIE-1⁻) and the amountof mouse SCN1A RNA including (NIE-1⁺)
in wildtype mice, single dose

| | CORTEX | |
|---|---|---|
| | NIE-1⁻ | NIE-1⁺ |
| Compound No. | % control | % control |
| PBS | 100 | 100 |
| 1472450 | 154 | 8 |
| 1472451 | 169 | 3 |
| 1472452 | 171 | 9 |
| 1472453 | 158 | 2 |
| 1472454 | 139 | 8 |
| 1472455 | 156 | 4 |
| 1472456 | 170 | 4 |
| 1472457 | 178 | 2 |
| 1472458 | 197 | 4 |
| 1472459 | 199 | 12 |
| 1472460 | 178 | 2 |
| 1472461 | 185 | 5 |
| 1472462 | 192 | 6 |
| 1472463 | 197 | 4 |
| 1472464 | 198 | 2 |
| 1472465 | 175 | 4 |
| 1472466 | 162 | 1 |
| 1472467 | 169 | 4 |
| 1472468 | 169 | 2 |
| 1472469 | 148 | 4 |
| 1472470 | 145 | 4 |
| 1472471 | 150 | 3 |
| 1472472 | 150 | 4 |
| 1472473 | 152 | 1 |

### Tolerability

At 3 hours post-injection, mice were evaluated according to seven different criteria. The criteria are (1) the mouse was bright, alert, and responsive; (2) the mouse was standing or hunched without stimuli; (3) the mouse showed any movement without stimuli; (4) the mouse demonstrated forward movement after it was lifted; (5) the mouse demonstrated any movement after it was lifted; (6) the mouse responded to tail pinching; (7) regular breathing. For each of the 7 criteria, a mouse was given a subscore of 0 if it met the criteria and 1 if it did not (the functional observational battery score or FOB). After all 7 criteria were evaluated, the scores were summed for each mouse and averaged within each treatment group. As shown in the data provided in Table 21 above, and Table 25 below, the compounds described in Table 23 are more tolerable than comparator compound 1367010 in this assay.

TABLE 25
Tolerability scores in mice

| Compound | FOB 3 |
|---|---|
| No. | hour |
| PBS | 0 |
| 1472450 | 3.33 |
| 1472451 | 3.00 |
| 1472452 | 2.33 |
| 1472453 | 1.00 |
| 1472454 | 2.33 |
| 1472455 | 1.33 |
| 1472456 | 1.00 |
| 1472457 | 2.67 |
| 1472458 | 1.33 |
| 1472459 | 1.67 |
| 1472460 | 1.67 |
| 1472461 | 2.00 |
| 1472462 | 2.67 |
| 1472463 | 2.67 |
| 1472464 | 2.00 |
| 1472465 | 3.67 |
| 1472466 | 2.67 |
| 1472467 | 3.67 |
| 1472468 | 3.33 |
| 1472469 | 2.67 |
| 1472470 | 2.67 |
| 1472471 | 3.00 |
| 1472472 | 2.67 |
| 1472473 | 3.33 |

### Example 10: Tolerability of Modified Oligonucleotides Complementary to Human SCN1A in Rats, 3 Hour Study

Modified oligonucleotides described in Table 23 above, and comparator compound 1367010, were tested in rats to assess the tolerability of the oligonucleotides. Sprague Dawley rats each received a single intrathecal (IT) dose of 3 mg of oligonucleotide listed in the table below. Each treatment group consisted of 4 rats. A group of 4 rats received PBS as a negative control. At 3 hours post-injection, movement in 7 different parts of the body were evaluated for each rat. The 7 body parts are (1) the rat's tail; (2) the rat's posterior posture; (3) the rat's hind limbs; (4) the rat's hind paws; (5) the rat's forepaws; (6) the rat's anterior posture; (7) the rat's head. For each of the 7 different body parts, each rat was given a sub-score of 0 if the body part was moving or 1 if the body part was paralyzed (the functional observational battery score or FOB). After each of the 7 body parts were evaluated, the sub-scores were summed for each rat and then averaged for each group. For example, if a rat's tail, head, and all other evaluated body parts were moving 3 hours after the 3 mg IT dose, it would get a summed score of 0. If another rat was not moving its tail 3 hours after the 3 mg IT dose but all other evaluated body parts were moving, it would receive a score of 1. Results are presented as the average score for each treatment group.

TABLE 26
Tolerability scores in rats (n = 4) at 3 mg dose

| Compound | 3 hr. |
|---|---|
| No. | FOB |
| PBS | 0.00 |
| 1367010 | 6.00 |

TABLE 27
Tolerability scores in rats (n = 4) at 3 mg dose

| Compound | 3 hr. |
|---|---|
| No. | FOB |
| PBS | 0.00 |
| 1472450 | 1.00 |
| 1472451 | 2.00 |
| 1472452 | 2.75 |
| 1472453 | 2.25 |
| 1472454 | 3.00 |
| 1472455 | 1.50 |
| 1472456 | 2.25 |
| 1472457 | 2.25 |
| 1472458 | 3.00 |
| 1472459 | 2.25 |
| 1472460 | 2.75 |
| 1472461 | 2.25 |
| 1472462 | 2.25 |
| 1472463 | 1.50 |
| 1472464 | 1.25 |
| 1472465 | 2.25 |
| 1472466 | 3.00 |
| 1472467 | 2.25 |
| 1472468 | 4.00 |
| 1472469 | 1.75 |
| 1472470 | 3.00 |
| 1472471 | 2.25 |
| 1472472 | 2.25 |
| 1472473 | 0.00 |

### Example 11: Tolerability of Modified Oligonucleotides Complementary to Human SCN1A in Rats, Long-Term Assessment

In separate studies run under the same conditions, modified oligonucleotides described in Table 23 and comparator compound 1367010 were tested in Sprague Dawley rats to assess long-term tolerability. Sprague Dawley rats each received a single intrathecal (IT) delivered dose of 3 mg of oligonucleotide or PBS. Beginning 1-week post-treatment, each animal was weighed and evaluated weekly by a trained observer for adverse events. Adverse events are defined as neurological dysfunction not typical in PBS-treated control animals, including, but not limited to: abnormal limb splay, abnormal gait, tremors, abnormal respiration, paralysis, and spasticity. The onset of the adverse event is defined as the week post-dosing when the dysfunction was first recorded. If no adverse event was achieved, there is no onset (−). If the animal died prior to 1-week due to acute toxicity, long term adverse effects could not be verified, and such cases are marked with a ‘Ø’ symbol. Similar tolerability assessments are described in Ostergaard et al., Nucleic Acids Res., 2013 Nov, 41(21), 9634-9650 and Southwell et al., Mol Ther., 2014 Dec, 22(12), 2093-2106.

At the end of the study, the rats are sacrificed and tissues were collected. Histopathology was performed on sections of cerebellum using calbindin stain. The calbindin stained cerebellum sections were evaluated for Purkinje cell loss. Purkinje cell loss was observed in calbindin stained cerebellum sections as indicated in the table below. Cerebellum and spinal cord were also evaluated using an antibody specific for modified oligonucleotides. Animals demonstrating no oligonucleotide uptake were excluded from histopathology analysis. Histology was not completed for animals that were sacrificed early due to adverse events. In cases where purkinje cell loss could not be evaluated due to death of mice in less than a week post treatment, the values are indicated as ‘N/A’. Additionally, cortical GFAP, a marker of astrogliosis (Abdelhak, et al., Scientific Reports, 2018, 8, 14798), was measured using RT-PCR, and average elevations >2-fold are noted below. In cases where GFAP levels could not be evaluated due to death of mice in less than a week post treatment, the values are indicated as ‘N/A’.

TABLE 28
Long-term tolerability in rats at 3 mg dose

| | Adverse | Purkinje | |
|---|---|---|---|
| | event | cell loss | |
| | onset, | (# | Cortex |
| | weeks | animals | GFAP |
| | post- | with | mRNA |
| | treatment, | loss/# | >2-fold |
| Compound | individual | animals | PBS |
| Number | animals | tested) | Control |
| PBS | -, -, -, - | 0/4 | 100 |
| 1367010 | θ, θ, θ, θ | N/A | N/A |
| 1472450 | -, -, -, - | 0/4 | 151 |
| 1472451 | -, 7, -, - | 2/4 | 128 |
| 1472452 | -, -, 7, - | 0/4 | 112 |
| 1472453 | -, -, -, - | 1/4 | 100 |
| 1472454 | -, -, -, - | 0/4 | 136 |
| 1472455 | -, -, -, - | 0/4 | 116 |
| 1472456 | -, -, -, 7 | 0/4 | 140 |
| 1472457 | -, -, -, - | 0/4 | 135 |
| 1472458 | -, -, -, 3 | 0/4 | 118 |
| 1472459 | -, -, -, - | 0/4 | 129 |
| 1472460 | -, -, -, - | 1/4 | 130 |
| 1472461 | -, -, -, - | 0/4 | 130 |
| 1472462 | -, -, -, - | 0/4 | 130 |
| 1472463 | -, -, -, - | 0/4 | 118 |
| 1472464 | -, -, -, - | 0/4 | 111 |
| 1472465 | -, -, -, - | 0/4 | 119 |
| 1472466 | 7, -, 7, 7 | 1/4 | 159 |
| 1472467 | -, -, -, - | 0/4 | 120 |
| 1472468 | -, -, - | 0/3 | 148 |
| 1472469 | -, -, -, - | 0/4 | 123 |
| 1472470 | -, -, -, - | 0/4 | 150 |
| 1472471 | -, -, -, - | 0/4 | 142 |
| 1472472 | -, -, -, - | 0/4 | 125 |
| 1472473 | -, -, -, - | 0/4 | 115 |

### Example 12: Design of Modified Oligonucleotides Complementary to a Human SCN1A Nucleic Acid

Modified oligonucleotides complementary to a human SCN1A nucleic acid were designed and synthesized as indicated in Table 29 below.

The modified oligonucleotides in Table 29 are 18 nucleosides in length. Each nucleoside comprises a 2′-NMA sugar moiety. The internucleoside linkages throughout each modified oligonucleotide are phosphorothioate internucleoside linkages. All cytosine nucleobases throughout each modified oligonucleotide are 5-methylcytosines.

Each modified oligonucleotide listed in Table 29 below is 100% complementary to SEQ ID NO: 1 (the complement of GENBANK Accession No. NC_000002.12 truncated from nucleotides 165982001 to 166152000). “Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence.

TABLE 29
2′-NMA modified oligonucleotides with uniform
phosphorothioate internucleoside linkages

| | | SEQ | SEQ | |
|---|---|---|---|---|
| | | ID No: | ID No: | SEQ |
| Compound | Nucleobase Sequence | 1 Start | 1 Stop | ID |
| Number | (5′ to 3′) | Site | Site | No. |
| 1521407 | GGTAGCAAAAGGGGTAAT | 144779 | 144796 | 881 |
| 1521408 | AGCAAAAGGGGTAATACA | 144776 | 144793 | 885 |
| 1521409 | GCAAAAGGGGTAATACAG | 144775 | 144792 | 888 |
| 1521410 | CAAAAGGGGTAATACAGT | 144774 | 144791 | 882 |
| 1521411 | AAAGGGGTAATACAGTAC | 144772 | 144789 | 880 |
| 1521412 | AAGGGGTAATACAGTACC | 144771 | 144788 | 883 |
| 1521413 | GTAATACAGTACCCATAA | 144766 | 144783 | 511 |
| 1521414 | TAATACAGTACCCATAAT | 144765 | 144782 | 750 |
| 1521415 | AATACAGTACCCATAATA | 144764 | 144781 | 736 |
| 1521416 | ATACAGTACCCATAATAA | 144763 | 144780 | 642 |
| 1521417 | TACAGTACCCATAATAAA | 144762 | 144779 | 519 |
| 1521418 | ACAGTACCCATAATAAAG | 144761 | 144778 | 741 |
| 1521419 | CCCATCCAAGTTGGAGCA | 144716 | 144733 | 664 |
| 1521420 | CCATCCAAGTTGGAGCAA | 144715 | 144732 | 645 |
| 1521421 | CATCCAAGTTGGAGCAAG | 144714 | 144731 | 550 |
| 1521422 | ATCCAAGTTGGAGCAAGA | 144713 | 144730 | 814 |
| 1521423 | TCCAAGTTGGAGCAAGAT | 144712 | 144729 | 692 |
| 1521424 | CCAAGTTGGAGCAAGATT | 144711 | 144728 | 587 |
| 1521425 | CAAGTTGGAGCAAGATTA | 144710 | 144727 | 523 |
| 1521426 | AAGTTGGAGCAAGATTAT | 144709 | 144726 | 778 |
| 1521428 | TTGGAGCAAGATTATCCT | 144706 | 144723 | 510 |
| 1521429 | TGGAGCAAGATTATCCTA | 144705 | 144722 | 816 |
| 1521430 | GGAGCAAGATTATCCTAT | 144704 | 144721 | 696 |
| 1521431 | GAGCAAGATTATCCTATA | 144703 | 144720 | 590 |

### Example 13: Activity of Modified Oligonucleotides Targeting SCN1A in Wildtype Mice

Wildtype C571BL/6 mice were divided into groups of 3 mice each. Each mouse received a single ICV bolus of 50 μg of modified oligonucleotide. A group of 4 mice received PBS as a negative control.

Compound No. 1367010, previously described in WO 2019/040923 and herein above, was added as a comparator compound.

Two weeks post treatment mice were sacrificed and RNA was extracted from cortical brain tissue for real-time qPCR analysis of SCN1A RNA using primer probe set RTS48951 (described herein above) to measure the amount of SCN1A RNA that excludes the mouse form of NIE-1 (NIE-1⁻) and primer probe set RTS48869 (forward sequence GCTCAAGCTCATCTCGCT, designated herein as SEQ ID NO: 27; reverse sequence AGCTCCGCAAGAAACATCC, designated herein as SEQ ID NO: 28: probe sequence TTCGATTTTGTGGTGGTCATCCTCTCC, designated herein as SEQ ID NO; 29) to measure the total amount of SCN1A mRNA transcript. SCN1A RNA is presented as % o of the average of the PBS control (% control), normalized to mouse GAPDH. Mouse GAPDH was amplified using primer probe set mGapdh_LTS00102 (described herein above).

TABLE 30
Effect of modified oligonucleotides on the amount of mouse SCN1A
excluding NIE-1 (NIE-1⁻) and the amount of total mouse SCN1A mRNA
in wildtype mice, single dose

| | CORTEX | |
|---|---|---|
| | | Total |
| | | SCN1A |
| Compound No. | NIE-1⁻ | mRNA |
| PBS | 100 | 100 |
| 1367010 | 120 | 126 |
| 1429226 | 142 | 142 |
| 1521407 | 129 | 130 |
| 1521408 | 131 | 121 |
| 1521409 | 126 | 112 |
| 1521410 | 135 | 118 |
| 1521411 | 125 | 11 |
| 1521412 | 123 | 117 |
| 1521413 | 117 | 116 |
| 1521414 | 103 | 114 |
| 1521415 | 143 | 132 |
| 1521416 | 113 | 111 |
| 1521417 | 106 | 104 |
| 1521418 | 110 | 105 |
| 1521419 | 125 | 128 |
| 1521420 | 114 | 119 |
| 1521421 | 134 | 127 |
| 1521422 | 116 | 112 |
| 1521423 | 132 | 122 |
| 1521424 | 120 | 117 |
| 1521425 | 128 | 136 |
| 1521426 | 119 | 126 |
| 1521428 | 119 | 121 |
| 1521429 | 129 | 120 |
| 1521430 | 117 | 116 |
| 1521431 | 111 | 116 |

1. An oligomeric compound comprising a modified oligonucleotide consisting of 12 to 30 linked nucleosides wherein the nucleobase sequence of the modified oligonucleotide is at least 85% complementary to an equal length portion of a SCN1A nucleic acid, and wherein the modified oligonucleotide comprises at least one modification selected from a modified sugar moiety and a modified internucleoside linkage.

2. An oligomeric compound comprising a modified oligonucleotide consisting of 12 to 30 linked nucleosides and having a nucleobase sequence comprising at least 12, at least 13, at least 14, at least 15, at least 16, at least 17, or 18 contiguous nucleobases of any of the nucleobase sequences of SEQ ID NOs 41-889, wherein the modified oligonucleotide comprises at least one modification selected from a modified sugar moiety and a modified intemucleoside linkage.

3. The oligomeric compound of claim 1 or claim 2, wherein the modified oligonucleotide has a nucleobase sequence that is at least 90%, 95%, or 100% complementary to the nucleobase sequence of SEQ ID NO: 1 or SEQ ID NO: 2 when measured across the entire nucleobase sequence of the modified oligonucleotide.

4. The oligomeric compound of any of claims 1-3, wherein the modified oligonucleotide has a nucleobase sequence comprising a portion of at least 8, at least 9, at least 10, at least 11, at least 12, at least 13, at least 14, at least 15, at least 16, at least 17, or at least 18 contiguous nucleobases, wherein the portion is complementary to SEQ ID NO: 13.

5. The oligomeric compound of any of claims 1-4 wherein the modified oligonucleotide comprises at least one modified sugar moiety.

6. The oligomeric compound of claim 5, wherein the modified oligonucleotide comprises at least one bicyclic sugar moiety.

7. The oligomeric compound of claim 6, wherein the bicyclic sugar moiety has a 4′-2′ bridge, wherein the 4′-2′ bridge is selected from —CH₂—O—; and —CH(CH₃)—O—.

8. The oligomeric compound of any of claims 5-7, wherein the modified oligonucleotide comprises at least one non-bicyclic modified sugar moiety.

9. The oligomeric compound of claim 8, wherein the non-bicyclic modified sugar moiety is any of a 2′-MOE sugar moiety, a 2′-NMA sugar moiety, a 2′-OMe sugar moiety, or a 2′-F sugar moiety.

10. The oligomeric compound any of claims 5-9, wherein the modified oligonucleotide comprises at least one sugar surrogate.

11. The oligomeric compound of claim 10, wherein the sugar surrogate is any of morpholino, modified morpholino, PNA, THP, and F-HNA.

12. The oligomeric compound of claim 5, wherein each nucleoside of the modified oligonucleotide comprises a modified sugar moiety.

13. The oligomeric compound of claim 12, wherein each modified sugar moiety is a 2′-MOE sugar moiety.

14. The oligomeric compound of claim 12, wherein each modified sugar moiety is a 2′-NMA sugar moiety.

15. The oligomeric compound of any of claims 1-14, wherein the modified oligonucleotide comprises at least one modified intemucleoside linkage.

16. The oligomeric compound of any of claims 1-14, wherein each intemucleoside linkage of the modified oligonucleotide is a modified intemucleoside linkage.

17. The oligomeric compound of claim 15 or claim 16, wherein at least one modified intemucleoside linkage is a phosphorothioate intemucleoside linkage.

18. The oligomeric compound of claim 15 or claim 17 wherein the modified oligonucleotide comprises at least one phosphodiester intemucleoside linkage.

19. The oligomeric compound of any of claims 15, 17, or 18, wherein each intemucleoside linkage is independently selected from a phosphodiester intemucleoside linkage and a phosphorothioate intemucleoside linkage.

20. The oligomeric compound of claim 16, wherein each intemucleoside linkage is a phosphorothioate internucleoside linkage.

21. The oligomeric compound of any of claims 1-20, wherein the modified oligonucleotide comprises at least one modified nucleobase.

22. The oligomeric compound of claim 21, wherein the modified nucleobase is a 5-methyl cytosine.

23. The oligomeric compound of any of claims 1-22, wherein the modified oligonucleotide consists of 12-22, 12-20, 14-20, 15-25, 16-20, 18-22 or 18-20 linked nucleosides.

24. The oligomeric compound of any of claims 1-23, wherein the modified oligonucleotide consists of 16, 17, or 18 linked nucleosides.

25. An oligomeric compound comprising a modified oligonucleotide according to the following chemical notation: Aₙₛ Gₙₛ Tₙₛ Tₙₛ Tₙₛ Gₙₛ Aₙₛ Gₙₛ ^{m}Cₙₛ Aₙₛ Aₙₛ Gₙₛ Aₙₛ Tₙₛ Tₙₛ Aₙₛ Tₙₛ ^{m}Cₙ (SEQ ID NO: 41), wherein,
A=an adenine nucleobase,
^{m}C=a 5-methyl cytosine nucleobase,
G=a guanine nucleobase,
T=a thymine nucleobase,
n=a 2′-NMA sugar moiety, and
s=a phosphorothioate internucleoside linkage.

26. An oligomeric compound comprising a modified oligonucleotide according to any one of the following chemical notations (5′ to 3′):
i)
(SEQ ID NO: 41)
AₑₛGₑₒTₑₒTₑₛGₑₛGₑₛAₑₛGₑₛ^{m}CₑₛAₑₛAₑₛGₑₛAₑₛTₑₛTₑₛAₑₛTₑₛ^{m}Cₑ;
ii)
(SEQ ID NO: 41)
AₑₛGₑₒTₑₛTₑₒGₑₛGₑₛAₑₛGₑₛ^{m}CₑₛAₑₛAₑₛGₑₛAₑₛTₑₛTₑₛAₑₛTₑₛ^{m}Cₑ;
iii)
(SEQ ID NO: 41)
AₑₛGₑₒTₑₛTₑₛGₑₛGₑₒAₑₛGₑₛ^{m}CₑₛAₑₛAₑₛGₑₛAₑₛTₑₛTₑₛAₑₛTₑₛ^{m}Cₑ;
iv)
(SEQ ID NO: 41)
AₑₛGₑₒTₑₛTₑₛGₑₛGₑₛAₑₛGₑₒ^{m}CₑₛAₑₛAₑₛGₑₛAₑₛTₑₛTₑₛAₑₛTₑₛ^{m}Cₑ;
v)
(SEQ ID NO: 41)
AₑₛGₑₛTₑₛTₑₒGₑₒGₑₛAₑₛGₑₛ^{m}CₑₛAₑₛAₑₛGₑₛAₑₛTₑₛTₑₛAₑₛTₑₛ^{m}Cₑ;
vi)
(SEQ ID NO: 41)
AₑₛGₑₛTₑₛTₑₛGₑₛGₑₛAₑₛGₑₒ^{m}CₑₒAₑₛAₑₛGₑₛAₑₛTₑₛTₑₛAₑₛTₑₛ^{m}Cₑ;
vii)
(SEQ ID NO: 41)
AₑₛGₑₛTₑₛTₑₛGₑₛGₑₛAₑₛGₑₛ^{m}CₑₛAₑₒAₑₒGₑₛAₑₛTₑₛTₑₛAₑₛTₑₛ^{m}Cₑ;
viii)
(SEQ ID NO: 41)
AₑₛGₑₛTₑₛTₑₛGₑₛGₑₛAₑₛGₑₛ^{m}CₑₛAₑₛAₑₛGₑₒAₑₒTₑₛTₑₛAₑₛTₑₛ^{m}Cₑ;
ix)
(SEQ ID NO: 41)
AₑₛGₑₒTₑₛTₑₛGₑₛGₑₛAₑₛGₑₛ^{m}CₑₛAₑₒAₑₛGₑₛAₑₛTₑₛTₑₛAₑₛTₑₛ^{m}Cₑ;
x)
(SEQ ID NO: 41)
AₑₛGₑₒTₑₛTₑₛGₑₛGₑₛAₑₛGₑₛ^{m}CₑₛAₑₛAₑₛGₑₒAₑₛTₑₛTₑₛAₑₛTₑₛ^{m}Cₑ;
xi)
(SEQ ID NO: 41)
AₑₛGₑₒTₑₛTₑₛGₑₛGₑₛAₑₛGₑₛ^{m}CₑₛAₑₛAₑₛGₑₛAₑₛTₑₛTₑₒAₑₛTₑₛ^{m}Cₑ;
xii)
(SEQ ID NO: 41)
AₑₛGₑₛTₑₒTₑₛGₑₛGₑₛAₑₛGₑₛ^{m}CₑₛAₑₛAₑₛGₑₛAₑₛTₑₛTₑₒAₑₛTₑₛ^{m}Cₑ;
xiii)
(SEQ ID NO: 41)
AₑₛGₑₛTₑₛTₑₛGₑₒGₑₛAₑₛGₑₛ^{m}CₑₛAₑₛAₑₛGₑₛAₑₛTₑₛTₑₒAₑₛTₑₛ^{m}Cₑ;
xiv)
(SEQ ID NO: 41)
AₑₛGₑₛTₑₛTₑₛGₑₛGₑₒAₑₒGₑₛ^{m}CₑₛAₑₛAₑₛGₑₛAₑₛTₑₛTₑₛAₑₛTₑₛ^{m}Cₑ;
xv)
(SEQ ID NO: 41)
AₑₛGₑₛTₑₛTₑₛGₑₛGₑₛAₑₒGₑₛ^{m}CₑₛAₑₛAₑₛGₑₛAₑₛTₑₛTₑₒAₑₛTₑₛ^{m}Cₑ;
xvi)
(SEQ ID NO: 41)
AₑₛGₑₛTₑₛT_{eS}GₑₛGₑₛA_{eS}Gₑₛ^{m}CₑₒAₑₛAₑₛGₑₛAₑₛTₑₒTₑₛAₑₛTₑₛ^{m}Cₑ;
xvii)
(SEQ ID NO: 41)
AₑₛGₑₛTₑₛTₑₛGₑₛGₑₛAₑₛGₑₛ^{m}CₑₒAₑₛAₑₛGₑₛAₑₛTₑₛTₑₒAₑₛTₑₛ^{m}Cₑ;
xviii)
(SEQ ID NO: 41)
AₑₛGₑₛTₑₛTₑₛGₑₛGₑₛAₑₛGₑₛ^{m}CₑₛAₑₛAₑₒGₑₛAₑₛTₑₛTₑₒAₑₒTₑₛ^{m}Cₑ;
xix)
(SEQ ID NO: 41)
AₑₛGₑₛTₑₛTₑₛGₑₛGₑₛAₑₛGₑₛ^{m}CₑₛAₑₛAₑₛGₑₒAₑₛTₑₒTₑₛAₑₛTₑₛ^{m}Cₑ;
xx)
(SEQ ID NO: 41)
AₑₛGₑₛTₑₛTₑₛGₑₛGₑₛAₑₛGₑₛ^{m}CₑₛAₑₛAₑₛGₑₛAₑₒTₑₛTₑₒAₑₛTₑₛ^{m}Cₑ;
xxi)
(SEQ ID NO: 41)
AₑₛGₑₛTₑₛTₑₛGₑₛGₑₛAₑₛGₑₛ^{m}CₑₛAₑₛAₑₛGₑₛAₑₛTₑₒTₑₒAₑₛTₑₛ^{m}Cₑ;
xxii)
(SEQ ID NO: 890)
AₑₒAₑₛGₑₛTₑₛTₑₛGₑₛGₑₛAₑₛGₑₛ^{m}CₑₛAₑₛAₑₛGₑₛAₑₛTₑₛTₑₛAₑₛTₑₛ^{m}Cₑₒ^{m}Cₑ;
xxiii)
(SEQ ID NO: 891)
AₑₛGₑₛTₑₛTₑₛGₑₛGₑₛAₑₛGₑₛ^{m}CₑₛAₑₛAₑₛGₑₛAₑₛTₑₛTₑₛAₑₛTₑₛmCₑₒ^{m}Cₑ;
xxiv)
(SEQ ID NO: 892)
AₑₒAₑₛGₑₛTₑₛTₑₛGₑₛGₑₛAₑₛGₑₛ^{m}CₑₛAₑₛAₑₛGₑₛAₑₛTₑₛTₑₛAₑₛTₑₛ^{m}Cₑ;
xxv)
(SEQ ID NO: 881)
GₙₛGₙₛTₙₛAₙₛGₙₛ^{m}CₙₛAₙₛAₙₛAₙₛAₙₛGₙₛGₙₛGₙₛGₙₛTₙₛAₙₛAₙₛTₙ;
xxvi)
(SEQ ID NO: 885)
AₙₛGₙₛ^{m}CₙₛAₙₛAₙₛAₙₛAₙₛGₙₛGₙₛGₙₛGₙₛTₙₛAₙₛAₙₛTₙₛAₙₛmCₙₛAₙ;
xxvii)
(SEQ ID NO: 888)
Gₙₛ^{m}CₙₛAₙₛAₙₛAₙₛAₙₛGₙₛGₙₛGₙₛGₙₛTₙₛAₙₛAₙₛTₙₛAₙₛ^{m}C_{nS}AₙₛGₙ;
xxviii)
(SEQ ID NO: 882)
^{m}CₙₛAₙₛAₙₛAₙₛAₙₛGₙₛGₙₛGₙₛGₙₛTₙₛAₙₛAₙₛTₙₛAₙₛ^{m}CₙₛAₙₛGₙₛTₙ;
xxix)
(SEQ ID NO: 880)
AₙₛAₙₛAₙₛGₙₛGₙₛGₙₛGₙₛTₙₛAₙₛAₙₛTₙₛAₙₛ^{m}CₙₛAₙₛGₙₛTₙₛAₙₛ^{m}Cₙ;
xxx)
(SEQ ID NO: 883)
AₙₛAₙₛGₙₛGₙₛGₙₛGₙₛTₙₛAₙₛAₙₛTₙₛAₙₛ^{m}CₙₛAₙₛGₙₛTₙₛAₙₛ^{m}Cₙₛ^{m}Cₙ;
xxxi)
(SEQ ID NO: 511)
GₙₛTₙₛAₙₛAₙₛTₙₛAₙₛ^{m}CₙₛAₙₛGₙₛTₙₛAₙₛ^{m}Cₙₛ^{m}Cₙₛ^{m}CₙₛAₙₛTₙₛAₙₛAₙ;
xxxii)
(SEQ ID NO: 750)
TₙₛAₙₛAₙₛTₙₛAₙₛ^{m}CₙₛAₙₛGₙₛTₙₛAₙₛ^{m}Cₙₛ^{m}Cₙₛ^{m}CₙₛAₙₛTₙₛAₙₛAₙₛTₙ;
xxxiii)
(SEQ ID NO: 736)
AₙₛAₙₛTₙₛAₙₛ^{m}CₙₛAₙₛGₙₛTₙₛAₙₛ^{m}Cₙₛ^{m}Cₙₛ^{m}CₙₛAₙₛTₙₛAₙₛAₙₛTₙₛAₙ;
xxxiv)
(SEQ ID NO: 642)
AₙₛTₙₛAₙₛ^{m}CₙₛAₙₛGₙₛTₙₛAₙₛ^{m}Cₙₛ^{m}Cₙₛ^{m}CₙₛAₙₛTₙₛAₙₛAₙₛTₙₛAₙₛAₙ;
xxxv)
(SEQ ID NO: 519)
TₙₛAₙₛ^{m}CₙₛAₙₛGₙₛTₙₛAₙₛ^{m}Cₙₛ^{m}Cₙₛ^{m}CₙₛAₙₛTₙₛAₙₛAₙₛTₙₛAₙₛAₙₛAₙ;
xxxvi)
(SEQ ID NO: 741)
Aₙₛ^{m}CₙₛAₙₛGₙₛTₙₛAₙₛ^{m}Cₙₛ^{m}Cₙₛ^{m}CₙₛAₙₛTₙₛAₙₛAₙₛTₙₛAₙₛAₙₛAₙₛGₙ;
xxxvii)
(SEQ ID NO: 664)
^{m}Cₙₛ^{m}Cₙₛ^{m}CₙₛAₙₛTₙₛ^{m}Cₙₛ^{m}CₙₛAₙₛAₙₛGₙₛTₙₛTₙₛGₙₛGₙₛAₙₛGₙₛ^{m}CₙₛAₙ;
xxxviii)
(SEQ ID NO: 645)
^{m}Cₙₛ^{m}CₙₛAₙₛTₙₛ^{m}CₙₛmCₙₛAₙₛAₙₛGₙₛTₙₛTₙₛGₙₛGₙₛAₙₛGₙₛ^{m}CₙₛAₙₛAₙ;
xxxix)
(SEQ ID NO: 550)
^{m}CₙₛAₙₛTₙₛ^{m}Cₙₛ^{m}CₙₛAₙₛAₙₛGₙₛTₙₛTₙₛGₙₛGₙₛAₙₛGₙₛ^{m}CₙₛAₙₛAₙₛGₙ;
xl)
(SEQ ID NO: 814)
AₙₛTₙₛ^{m}Cₙₛ^{m}CₙₛAₙₛAₙₛGₙₛTₙₛTₙₛGₙₛGₙₛAₙₛGₙₛ^{m}CₙₛAₙₛAₙₛGₙₛAₙ;
xli)
(SEQ ID NO: 692)
Tₙₛ^{m}Cₙₛ^{m}CₙₛAₙₛAₙₛGₙₛTₙₛTₙₛGₙₛGₙₛAₙₛGₙₛ^{m}CₙₛAₙₛAₙₛGₙₛAₙₛTₙ;
xlii)
(SEQ ID NO: 587)
^{m}Cₙₛ^{m}CₙₛAₙₛAₙₛGₙₛTₙₛTₙₛGₙₛGₙₛAₙₛGₙₛ^{m}CₙₛAₙₛAₙₛGₙₛAₙₛTₙₛTₙ;
xliii)
(SEQ ID NO: 523)
^{m}CₙₛAₙₛAₙₛGₙₛTₙₛTₙₛGₙₛGₙₛAₙₛGₙₛ^{m}CₙₛAₙₛAₙₛGₙₛAₙₛTₙₛTₙₛAₙ;
xliv)
(SEQ ID NO: 778)
AₙₛAₙₛGₙₛTₙₛTₙₛGₙₛGₙₛAₙₛGₙₛ^{m}CₙₛAₙₛAₙₛGₙₛAₙₛTₙₛTₙₛAₙₛTₙ;
xlv)
(SEQ ID NO: 510)
TₙₛTₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙ;
xlvi)
(SEQ ID NO: 816)
TₙₛGₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙ;
xlvii)
(SEQ ID NO: 696)
GₙₛGₙₛAₙₛGₙₛ^{m}CₙₛAₙₛAₙₛGₙₛAₙₛTₙₛTₙₛAₙₛTₙₛ^{m}Cₙₛ^{m}CₙₛTₙₛAₙₛTₙ;
or
xlviii)
(SEQ ID NO: 590)
GₙₛAₙₛG_{nS}^{m}C_{nS}AₙₛAₙₛGₙₛAₙₛTₙₛTₙₛAₙₛTₙₛ^{m}Cₙₛ^{m}C_{nS}TₙₛAₙₛTₙₛAₙ,
wherein,
A=an adenine nucleobase,
^{m}C=a 5-methyl cytosine nucleobase,
G=a guanine nucleobase,
T=a thymine nucleobase,
e=a 2′-MOE sugar moiety,
n=a 2′-NMA sugar moiety,
o=a phosphodiester internucleoside linkage, and
s=a phosphorothioate intemucleoside linkage.

27. The oligomeric compound of any of claims 1-26, wherein the oligomeric compound is a singled-stranded oligomeric compound.

28. The oligomeric compound of any of claims 1-27 consisting of the modified oligonucleotide.

29. The oligomeric compound of any of claims 1-28 comprising a conjugate group comprising a conjugate moiety and a conjugate linker.

30. The oligomeric compound of claim 29, wherein the conjugate group comprises a GalNAc cluster comprising 1-3 GalNAc ligands.

31. The oligomeric compound of claim 29 or claim 30, wherein the conjugate linker consists of a single bond.

32. The oligomeric compound of claim 29, wherein the conjugate linker is cleavable.

33. The oligomeric compound of claim 29, wherein the conjugate linker comprises 1-3 linker-nucleosides.

34. The oligomeric compound of any of claims 29-33, wherein the conjugate group is attached to the modified oligonucleotide at the 5′-end of the modified oligonucleotide.

35. The oligomeric compound of any of claims 29-33, wherein the conjugate group is attached to the modified oligonucleotide at the 3′-end of the modified oligonucleotide.

36. The oligomeric compound of any of claims 1-27 or 29-35 comprising a terminal group.

37. The oligomeric compound of any of claims 1-32 or 34-35, wherein the oligomeric compound does not comprise linker-nucleosides.

38. A modified oligonucleotide according to the following chemical structure: 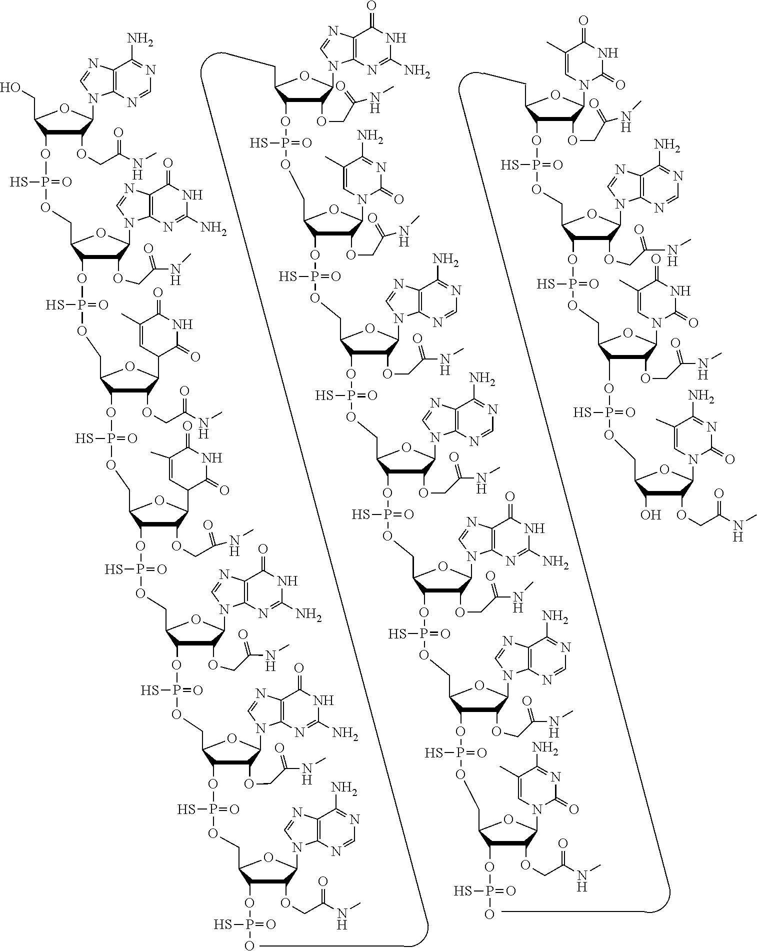
(SEQ ID NO: 41) or a salt thereof.

39. The modified oligonucleotide of claim 38, which is the sodium salt or the potassium salt.

40. A modified oligonucleotide according to the following chemical structure: 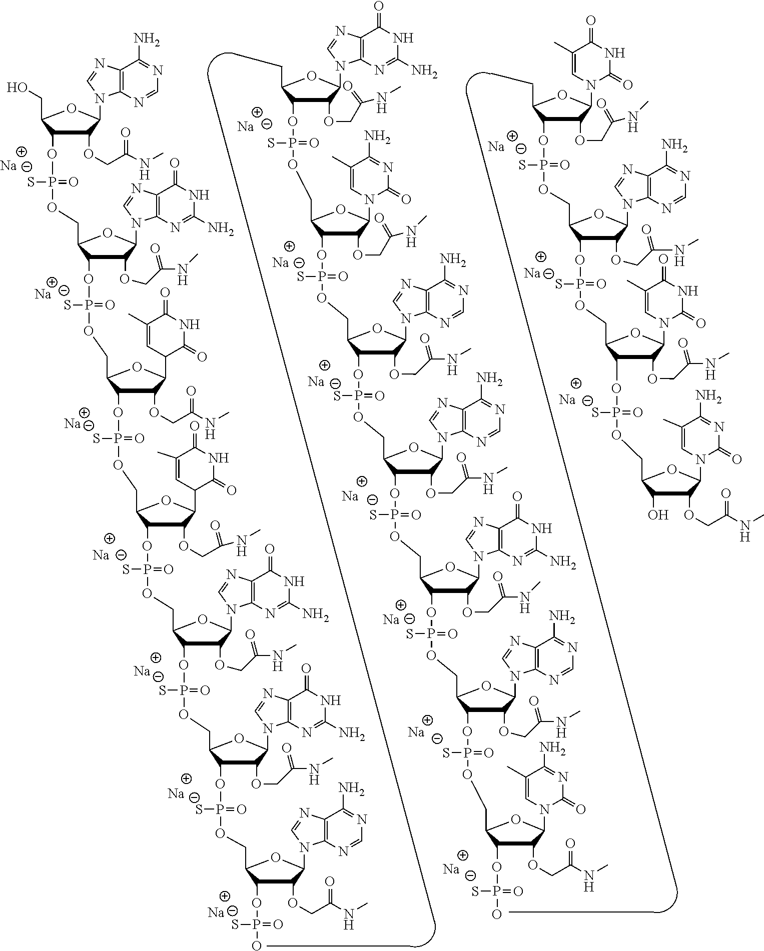
(SEQ ID NO: 41).

41. A chirally enriched population of modified oligonucleotides of any of claims 38-40, wherein the population is enriched for modified oligonucleotides comprising at least one particular phosphorothioate internucleoside linkage having a particular stereochemical configuration.

42. The chirally enriched population of claim 41, wherein the population is enriched for modified oligonucleotides comprising at least one particular phosphorothioate internucleoside linkage having the (Sp) configuration.

43. The chirally enriched population of claim 41, wherein the population is enriched for modified oligonucleotides comprising at least one particular phosphorothioate intemucleoside linkage having the (Rp) configuration.

44. The chirally enriched population of claim 41, wherein the population is enriched for modified oligonucleotides having a particular, independently selected stereochemical configuration at each phosphorothioate intemucleoside linkage.

45. The chirally enriched population of claim 44, wherein the population is enriched for modified oligonucleotides having the (Sp) configuration at each phosphorothioate intemucleoside linkage or for modified oligonucleotides having the (Rp) configuration at each phosphorothioate intemucleoside linkage.

46. The chirally enriched population of claim 44, wherein the population is enriched for modified oligonucleotides having the (Rp) configuration at one particular phosphorothioate internucleoside linkage and the (Sp) configuration at each of the remaining phosphorothioate internucleoside linkages.

47. The chirally enriched population of claim 44, wherein the population is enriched for modified oligonucleotides having at least 3 contiguous phosphorothioate internucleoside linkages in the Sp, Sp, and Rp configurations, in the 5′ to 3′ direction.

48. A population of modified oligonucleotides of any of claims 38-40, wherein all of the phosphorothioate internucleoside linkages of the modified oligonucleotide are stereorandom.

49. A pharmaceutical composition comprising the oligomeric compound of any of claims 1-37, the modified oligonucleotide of any of claims 38-40, the chirally-enriched population of any of claims 41-47, or the population of modified oligonucleotides of claim 48, and a pharmaceutically acceptable diluent or carrier.

50. The pharmaceutical composition of claim 49, comprising a pharmaceutically acceptable diluent and wherein the pharmaceutically acceptable diluent is artificial CSF (aCSF) or PBS.

51. The pharmaceutical composition of claim 50, wherein the pharmaceutical composition consists essentially of the modified oligonucleotide and artificial CSF (aCSF).

52. The pharmaceutical composition of claim 50, wherein the pharmaceutical composition consists essentially of the modified oligonucleotide and PBS.

53. A method of modulating splicing of an SCN1A RNA in a cell comprising contacting the cell with an oligomeric compound of any of claims 1-37, a modified oligonucleotide of any of claims 38-40, a chirally-enriched population of any of claims 41-47, a population of modified oligonucleotides of claim 48, or a pharmaceutical composition of any of claims 49-52.

54. The method of claim 53, wherein the amount of SCN1A RNA that includes an NIE is reduced.

55. The method of claim 54, wherein the amount of SCN1A RNA that includes NIE-1 is reduced.

56. The method of any of claims 53-55, wherein the amount of SCN1A RNA that excludes an NIE is increased.

57. The method of any of claims 53-56, wherein the amount of SCN1A RNA that excludes NIE-1 is increased.

58. A method of increasing the amount of full-length SCN1A RNA in a cell, comprising contacting the cell with an oligomeric compound of any of claims 1-37, a modified oligonucleotide of any of claims 38-40, a chirally-enriched population of any of claims 41-47, a population of modified oligonucleotides of claim 48, or a pharmaceutical composition of any of claims 49-52.

59. A method of increasing SCN1A RNA lacking NIE-1 in a cell, tissue, or organ, comprising contacting a cell, tissue, or organ with an oligomeric compound of any of claims 1-37, a modified oligonucleotide of any of claims 38-40, a chirally-enriched population of any of claims 41-47, a population of modified oligonucleotides of claim 48, or a pharmaceutical composition of any of claims 49-52.

60. The method of any of claims 53-59, wherein the cell is in vitro.

61. The method of any of claims 53-59, wherein the cell is in an animal.

62. A method of ameliorating a disease associated with SCN1A comprising administering to a subject having or at risk for developing a disease associated with SCN1A a therapeutically effective amount of a pharmaceutical composition according to any of claims 49-52, and thereby treating the disease associated with SCN1A.

63. The method of claim 62, comprising identifying a subject having or at risk for developing a disease associated SCN1A.

64. The method of claim 62 or claim 63, wherein the disease associated with SCN1A is a developmental or epileptic encephalopathic disease.

65. The method of claim 64, wherein the developmental or epileptic encephalopathic disease is Dravet Syndrome.

66. The method of claim 64, wherein the developmental or epileptic encephalopathic disease is any of Genetic Epilepsy with Febrile Seizures Plus (GEFS+), febrile seizures, Idiopathic/Generic Generalized Epilepsies (IGE/GGE), Temporal Lobe Epilepsy, Myoclonic Astatic Epilepsy (MAE), Lennox-Gastaut Syndrome, or Migrating Partial Epilepsy of Infancy (MMPSI).

67. The method of any of claims 64-66, wherein at least one symptom of the developmental or epileptic encephalopathic disease is ameliorated.

68. The method of claim 67, wherein the symptom is any of seizures, behavioral and developmental delays, movement and balance dysfunctions, motor and cognitive dysfunctions, delayed language and speech, visual motor integration dysfunctions, visual perception dysfunctions, executive dysfunctions, growth and nutrition issues, sleeping difficulties, chronic infections, sensory integration disorders, or dysautonomia.

69. The method of claim 68, wherein the seizures are frequent or prolonged.

70. The method of claim 68 or claim 69, wherein the seizure is any of convulsive, myoclonic, absence, focal, obtundation status, or tonic.

71. The method of any of claims 62-70, wherein the pharmaceutical composition is administered to the central nervous system or systemically.

72. The method of claim 71, wherein the pharmaceutical composition is administered to the central nervous system and systemically.

73. The method of any of claims 62-71, wherein the pharmaceutical composition is administered any of intrathecally, systemically, subcutaneously, or intramuscularly.
